SEQUENCE LISTING (17 sequences)

SEQ ID NO: 1 - protein, 208 aa, Homo sapiens
MSTGGDFGNPLRKFKLVFLGEQSVGKTSLITRFMYDSFDNTYQATIGIDFLSKTMYLEDRTVRLQLWDTAGQERFRSLIPSYIRDSTVAVVVYDITNVNSFQQTTKWIDDVRTERGSDVIIMLVGNKTDLADKRQVSIEEGERKAKELNVMFIETSAKAGYNVKQLFRRVAAALPGMESTQDRSREDMIDIKLEKPQEQPVSEGGCSC

SEQ ID NO: 2 - DNA, 627 bp, Homo sapiens
atgtccacgggcggagacttcgggaatccgctgaggaaattcaagctggtgttcctgggggagcaaagcgttggaaagacatctttgatcaccagattcatgtatgacagttttgacaacacctatcaggcaacaattggcattgactttttatcaaaaactatgtacttggaggatcgaacagtacgattgcaattatgggacacagcaggtcaagagcggttcaggagcttgattcctagctacattcgtgactccactgtggcagttgttgtttatgatatcacaaatgttaactcattccagcaaactacaaagtggattgatgatgtcagaacagaaagaggaagtgatgttatcatcatgctagtaggaaataaaacagatcttgctgacaagaggcaagtgtcaattgaggagggagagaggaaagccaaagagctgaatgttatgtttattgaaactagtgcaaaagctggatacaatgtaaagcagctctttcgacgtgtagcagcagctttgccgggaatggaaagcacacaggacagaagcagagaagatatgattgacataaaactggaaaagcctcaggagcaaccagtcagtgaaggaggctgttcctgctaa

SEQ ID NO: 3 - protein, 565 aa, Homo sapiens
MHGRLKVKTSEEQAEAKRLEREQKLKLYQSATQAVFQKRQAGELDESVLELTSQILGANPDFATLWNCRREVLQQLETQKSPEELAALVKAELGFLESCLRVNPKSYGTWHHRCLLGRLPEPNWTRELELCARFLEVDERNFHCWDYRRFVATQAAVPPAEELAFTDSLITRNFSNYSSWHYRSCLLPQLHPQPDSGPQGRLPEDVLLKELELVQNAFFTDPNDQSAWFYHRWLLGRADPQDALRCLHVSRDEACLTVSFSRPLLVGSRMEILLLMVDDSPLIVEWRTPDGRNRPSHVWLCDLPAASLNDQLPQHTFRVIWTAGDVQKECVLLKGRQEGWCRDSTTDEQLFRCELSVEKSTVLQSELESCKELQELEPENKWCLLIILLMRALDPLLYEKETLQYFQTLKAVDPMRATYLDDLRSKFLLENSVLKMEYAEVRVLHLAHKDLTVLCHLEQLLLVTHLDLSHNRLRTLPPALAALRCLEVLQASDNAIESLDGVTNLPRLQELLLCNNRLQQPAVLQPLASCPRLVLLNLQGNPLCQAVGILEQLAELLPSVSSVLT

SEQ ID NO: 4 - protein, 331 aa, Homo sapiens
MGTPQKDVIIKSDAPDTLLLEKHADYIASYGSKKDDYEYCMSEYLRMSGIYWGLTVMDLMGQLHRMNREEILAFIKSCQHECGGISASIGHDPHLLYTLSAVQILTLYDSINVIDVNKVVEYVKGLQKEDGSFAGDIWGEIDTRFSFCAVATLALLGKLDAINVEKAIEFVLSCMNFDGGFGCRPGSESHAGQIYCCTGFLAITSQLHQVNSDLLGWWLCERQLPSGGLNGRPEKLPDVCYSWWVLASLKIIGRLHWIDREKLRNFILACQDEETGGFADRPGDMVDPFHTLFGIAGLSLLGEEQIKPVNPVFCMPEEVLQRVNVQPELVS

SEQ ID NO: 5 - protein, 648 aa, Homo sapiens
MADTLPSEFDVIVIGTGLPESIIAAACSRSGRRVLHVDSRSYYGGNWASFSFSGLLSWLKEYQENSDIVSDSPVWQDQILENEEAIALSRKDTIQHVEVFCYASQDLHEDVEEAGALQKNHALVTSANSTEAADSAFLPTEDESLSTMSCEMLTEQTPSSDPENALEVNGAEVTGEKENHCDDKTCVPSTSAEDMSENVPIAEDTTEQPKKNRITYSQIIEGRRFNIDLVSKLLYSRGLLIDLLIKSNVSRYAEFNITRILAFREGRVEQVPCSRADVFNSKQLTMVEKRMLMKFLTFCMEYEKYPDEYGYEEITFYEYLKTQKLTPNLQYIVMHSIAMTSETASSTIDGLKATKNFLHCLGRYGNTPFLFPLYGQGELPQCFCRMCAVFGGIYCLRHSVQCLVVDKESRKCKAIIDQFGQRIISEHFLVEDSYFPENMCSRVQYRQISRAVLITDRSVLTDSDQQISILTVPAEEPGTFAVRVIELCSSTMTCMKGTYLVHLTCTSSKTAREDLESVVQKLFVPYTEMEIENEQVEKPRILWALYFNMRDSSDISRSCYNDLPSNVYVCSGPDCGLGNDNAVKQAETLFQEICPNEDFCPPPPNPEDIILDGDSLQPEASESSAIPEANSETFKESTNLGNLEESSE

SEQ ID NO: 6 - DNA, 1944 bp, Homo sapiens
atggcggatactctcccttcggagtttgatgtgatcgtaatagggacgggtttgcctgaatccatcattgcagctgcatgttcaagaagtggccggagagttctgcatgttgattcaagaagctactatggaggaaactgggccagttttagcttttcaggactattgtcctggctaaaggaataccaggaaaacagtgacattgtaagtgacagtccagtgtggcaagccgatccttgaaaatgaagagccattgctcttagcaggaaggacaaaacattcaacatgtggaagtattttgttatgccagtcaggatttgcatgaagatgtcgaagaagctggtgcactgcagaaaaatcatgctcttgtgacatctgcaaactccacagaagctgcagattctgccttcctgcctacggaggatgagtcattaagcactatgagctgtgaaatgctcacagaacaaactccaagcagcgatccagagaatgcgctagaagtaaatggtgctgaagtgacaggggaaaaagaaaaccattgtgatgataaaacttgtgtgccatcaacttcagcagaagacatgagtgaaaatgtgcctatagcagaagataccacagagcaaccaaagaaaaacagaattacttactcacaaattattaaagaaggcaggagatttaatattgatttagtatcaaagctgctgtattctcgaggattactaattgatcttctaatcaaatctaatgttagtcgatatgcagagtttaaaaatattaccaggattcttgcatttcgagaaggcgagtggaacaggttccgtgttccggcgatgtctttagcaaacaacttactatggtagaaaagcgaatgctaatgaaatttcttacattttgtatggaatatgagaaatatcctgatgaatataaaggatatgaagagatcacatttttgaattttaaagactcaaaaattaacccccaacctccaatatattgtcatgcattcaattgcaatgacatcagagacagccagcagcaccatagatggtctcaaagctaccaaaaactttcttcactgtcttgggcggtatggcaacactccatttttgtttcctttatatggccaaggagaactcccccagtgtttctgcaggatgtgtgctgtgtttggtggaatttattgtcttcgccattcagtacagtgccttgtagtggacaaagaatccagaaaatgtaaagcaattatagatcagtttggtcagagaataatctctgagcatttcctcgtggaggacagttactttcctgagaacatgtgctcacgtgtgcaatacaggcagatctccagggcagtgctgattacagaagatctgtcctaaaaacagattcagatcaacgatttccttttgacagtgccagcagaggaaccaggaacttttgctgttcgggtcattgagttatgttcttcaacgatgacatgcatgaaaggcacctatttggttcatttgacttgcacatcttctaaaacagcaagagaagatttagaatcagttgtgcagaaattgtttgttccatatactgaaatggagatagaaaatgaacaagtagaaaagccaagaattctgtgggctctttacttcaatatgagagattcgtcagacatcagcaggagctgttataatgatttaccatccaacgtttatgtctgctctggcccagattgtggtttaggaaatgataatgcagtcaaacaggctgaaacacttttccaggaaatctgccccaatgaagatttctgtccccctccccaaatcctgaagacattatccttgatggagacagtttacagccagaggcttcagaatccagtgccataccagaggctaactcggagactttcaaggaaagcacaaaccttggaaacctagaggagtcctctgaaaa

SEQ ID NO: 7 - DNA, 1985 bp, Homo sapiens
gatatcgaattcctgcagcccggcggcaccatggcggatactctcccttcggagtttgatgtgatcgtaatagggacgggtttgcctgaatccatcattgcagctgcatgttcaagaagtggccggagagttctgcatgttgattcaagaagctactatggaggaaactgggccagttttagcttttcaggactattgtcctggctaaaggaataccaggaaaacagtgacattgtaagtgacagtccagtgtggcaagaccagatccttgaaaatgaagaagccattgctcttagcaggaaggacaaaactattcaacatgtggaagtattttgttatgccagtcaggatttgcatgaagatgtcgaagaagctggtgcactgcagaaaaatcatgctcttgtgacatctgcaaactccacagaagctgcagattctgccttcctgcctacggaggatgagtcattaagcactatgagctgtgaaatgctcacagaacaaactccaagcagcgatccagagaatgcgctagaagtaaatggtgctgaagtgacaggggaaaaagaaaaccattgtgatgataaaacttgtgtgccatcaacttcagcagaagacatgagtgaaaatgtgcctatagcagaagataccacagagcaaccaaagaaaaacgaattacttactcacaaatattaagaaggcaggagattaatattgatttagtatcaaagctgctgtattctcgaggattactaattgatcttctaatcaaatctaatgttagtcgatatgcagagtttaaaaatattaccaggattcttgcatttcgagaaggacgagtggaacaggttccgtgttccagagcagatgtctttaatagcaaacaacttactatggtagaaaagcgaatgctaatgaaatttcttacattttgtatggaatatgagaaatatcctgatgaatataaaggatatgaagagatcacattttatgaatatttaaagactcaaaaattaacccccaacctccaatatattgtcatgcattcaattgcaatgacatcagagacagccagcagcaccatagatggtctcaaagctaccaaaaactttcttcactgtcttgggcggtatggcaacactccatttttgtttcctttatatggccaaggagaactcccccagtgtttctgcaggatgtgtgctgtgtttggtggaatttattgtcttcgccattcagtacagtgccttgtagtggacaaagaatccagaaaatgtaaagcaattatagatcagtttggtcagagaataatctctgagcatttcctcgtggaggacagttactttcctgagaacatgtgctcacgtgtgcaatacaggcagatctccagggcagtgctgattacagatagatctgtcctaaaaacagattcagatcaacagatttccattttgacagtgccagcagaggaaccaggaacttttgctgttcgggtcattgagttatgttcttcaacgatgacatgctgaaaggcacctatttggttcatttgacttgcacatcttctaaaacagcaagagaagatttagaatcagttgtgcagaaattgtttgttccatatactgaaatggagatagaaaatgaacaagtagaaaagccaagaattctgtgggctctttacttcaatatgagagattcgtcagacatcagcaggagctgttataatgatttaccatccaacgtttatgtctgctctggcccagattgtggtttaggaaatgataatgcagtcaaacaggctgaaacacttttccaggaaatctgccccaatgaagatttctgtccccctccaccaaatcctgaagacattatccttgatggagacagtttacagccagaggcttcagaatccagtgccataccagaggctaactcggagactttcaggaaagcacaaaccttggaaacctagaggagtcctctgaaaa

SEQ ID NO: 8 - protein, 563 aa, Rattus norvegicus
MHGRLKVKTSEEQAEAKRLEREQKLKLYQSATQAVFQKRQAGELDESVLELTSQILGANPDFATLWNCRREVLQHLETEKSPEESAALVKAELGFLESCLRVNPKSYGTHHRCWLLSRLPEPNWARELELCARFLEADERNFHCWDYRRFVAAQAAVAPAEELAFTDSLITRNFSNYSSHYRSCLLPQLHPQPDSGPQGRLPENVLLKELELVQNAFFTDPNDQSAWFYHRLLGRAEPHDVLCCVHVSREEACLSVCFSRPLTVGSRMGTLLLMVDEAPLSVEWRTPDGRNRPSHVWLCDLPAASLNDQLPQHTFRVIWTGSDSQKECVLLKDRPECWCRDSATDEQLFRCELSVEKSTVLQSELESCKELQELEPENWCLLTIILLMRALDPLLYEKETLQYFSTLKAVDPMRAAYLDDLRSKFLLENSVLKMEYADVRVLHLAHKDLTVLCHLEQLLLVTHLDLSHNRLRALPPALAALRCLEVLQASDNALENVDGVANLPRLQELLLCNNRLQQSAAIQPLVSCPRLVLLNLQGNSLCQEEGIQERLAEMLPSVSSILT

SEQ ID NO: 9 - protein, 328 aa, Rattus norvegicus
MGTQQKDVTIKSDAPDTLLLEKHADYIASYGSKKDDYEYCMSEYLRMSGVYWGLTVMDLMGQLHRMNKEEILVFIKSCQHECGGVSASIGHDPHLLYTLSAVQILTLYDSIHVINVDKVVAYVQSLQEDGSFAGDIGEIDTRFSFCAVATLALLGKLDAINVEKAIEFVLSCMNFDGGFGCRPGSESHAGQIYCCTGFLAITSQLHQVNSDLLGWWLCERQLPSGGLNGRPEKLPDVCYSWWVLASLKIIGRLHIDREKLRSFILACQDEETGGFADRPGDMVDPFHTLFGIAGLSLLGEEQIKPVSPVFCMPEEVLQRVNVQPELVS

SEQ ID NO: 10 - protein, 567 aa, Homo sapiens
MHGRLKVKTSEEQAEAKRLEREQKLKLYQSATQAVFQKRQAGELDESVLELTSQILGANPDFATLWNCRREVLQQLETQKSPEELAALVKAELGFLESCLRVNPKSYGTWHHRCWLLGRLPEPNWTRELELCARFLEVDERNFHCWDYRRFVATQAAVPPAEELAFTDSLITRNFSNYSSWHYRSCLLPQLHPQPDSGPQGRLPEDVLLKELELVQNAFFTDPNDQSAWFYHRWLLGRADPQDALRCLHVSRDEACLTVSFSRPLLVGSRMEILLLMVDDSPLIVEWRTPDGRNRPSHVWLCDLPAASLNDQLPQHTFRVIWTAGDVQKECVLLKGRQEGWCRDSTTDEQLFRCELSVEKSTVLQSELESCKELQELEPENKWCLLTIILLMRALDPLLYEKETLQYFQTLKAVDPMRATYLDDLRSKFLLENSVLKMEYAEVRVLHLAHKDLTVLCHLEQLLLVTHLDLSHNRLRTLPPALAALRCLEVLQASDNAIESLDGVTNLPRLQELLLCNNRLQQPAVLQPLASCPRLVLLNLQGNPLCQAVGILEQLAELLPSVSSVLT

SEQ ID NO: 11 - protein, 567 aa, Rattus norvegicus
MHGRLKVKTSEEQAEAKRLEREQKLKLYQSATQAVFQKRQAGELDESVLELTSQILGANPDFATLWNCRREVLQHLETEKSPEESAALVKAELGFLESCLRVNPKSYGTWHHRCWLLSRLPEPNWARELELCARFLEADERNFHCWDYRRFVAAQAAVAPAEELAFTDSLITRNFSNYSSWHYRSCLLPQLHPQPDSGPQGRLPENVLLKELELVQNAFFTDPNDQSAWFYHRWLLGRAEPHDVLCCVHVSREEACLSVCFSRPLTVGSRMGTLLLMVDEAPLSVEWRTPDGRNRPSHVWLCDLPAASLNDQLPQHTFRVIWTGSDSQKECVLLKDRPECWCRDSATDEQLFRCELSVEKSTVLQSELESCKELQELEPENKWCLLTIILLMRALDPLLYEKETLQYFSTLKAVDPMRAAYLDDLRSKFLLENSVLKMEYADVRVLHLAHKDLTVLCHLEQLLLVTHLDLSHNRLRALPPALAALRCLEVLQASDNALENVDGVANLPRLQELLLCNNRLQQSAAIQPLVSCPRLVLLNLQGNSLCQEEGIQERLAEMLPSVSSILT

SEQ ID NO: 12 - protein, 331 aa, Rattus norvegicus
MGTQQKDVTIKSDAPDTLLLEKHADYIASYGSKKDDYEYCMSEYLRMSGVYWGLTVMDLMGQLHRMNKEEILVFIKSCQHECGGVSASIGHDPHLLYTLSAVQILTLYDSIHVINVDKVVAYVQSLQKEDGSFAGDIWGEIDTRFSFCAVATLALLGKLDAINVEKAIEFVLSCMNFDGGFGCRPGSESHAGQIYCCTGFLAITSQLHQVNSDLLGWWLCERQLPSGGLNGRPEKLPDVCYSWWVLASLKIIGRLHWIDREKLRSFILACQDEETGGFADRPGDMVDPFHTLFGIAGLSLLGEEQIKPVSPVFCMPEEVLQRVNVQPELVS

SEQ ID NO: 13 - DNA, 3419 bp, Homo sapiens
gcacgcacgcacgcacgccagcggccggcggggccgcaggctcgcgcccgggctcgccccgcgccgctccagaggctcgcgcactcagcaggttgggctgcggcggcggcggcagctgtggaagctcaggcgctgcgcgtgagaggtcccagatacgtctgcggttccggctccgccaccctcagcttctcttccccaggtctgggagccgagtgcggaaggagggaacggccctagctttgggaagccagaggacacccctggctcctgccgacaccgccctccttcccttcccagccgcgggcctcgctcggtgctaggctactctgccgggaggcggcggcggctgccagtctgtggagagtcctgctgccctccagccgggctcctccaccgggccttgcaggggccgagagagctcggtgcccgcccttccgctcgcctttttcgtcagctggctggagcagcatcggtccgggaggtctctaggctgaggcggcggccgctcctctagttccacaatgtccacgggcggagacttcgggaatccgctgaggaaattcaagctggtgttcctgggggagcaaagcgttggaaagacatctttgatcaccagattcatgtatgacagttttgacaacacctatcaggcaacaattggcattgactttttatcaaaaactatgtacttggaggatcgaacagtacgattgcaattatgggacacagcaggtcaagagcggttcaggagcttgattcctagctacattcgtgactccactgtggcagttgttgtttatgatatcacaaatgttaactcattccagcaaactacaaagtggattgatgatgtcagaacagaaagaggaagtgatgttatcatcatgctagtaggaaataaaacagatcttgctgacaagaggcaagtgtcaattgaggagggagagaggaaagccaaagagctgaatgttatgtttattgaaactagtgcaaaagctggatacaatgtaaagcagctctttcgacgtgtagcagcagctttgccgggaatggaaagcacacaggacagaagcagagaagatatgattgacataaaactggaaaagcctcaggagcaaccagtcagtgaaggaggctgttcctgctaatctcccatgtcatcttcaaccttcttcagaagctcactgctttggcccccttactctttcattgactgcagtgtgaatattggcttgaaccttttcccttcagtaataacgtattgcaattcatcattgctgcctgtctcgtggagatgatctattagcttcacaagcacaacaaaagtcagtgtcttcattatttatattttacaaaaagccaaaatatttcagcatattccagtgataactttaaaaattagatacattttcttaacatttttttcttttttaatgttatgataatgtacttcaaaatgatggaaatctcaacagtatgagtatggcttggttaacgagcggtatgttcacagcctactttatctctccttgcttttctcacctctcacttacccccattccctattaccctattcttacctagcctcccccgacttcctcaaaacaaacaagagatggcaaagcagcagttctaccaagcccattggaattatcctttaattttacagataccacttgctgtaggctacggaccaagatgtccaaaattattcttgagcactgatataaattacggtcttctttgaggtcaaaattcagccatcatggtaggcagtgcttgaatgagaaaaggctcctggtgcatcttcaaaatgagtcctaaagaacatactgagtacttagaagtagaagaacataagatgtatttctgactaaaacaaatggctctttcacatgtgctttattagactctgggagagaaaattaaccaagtgcttcagaacaggtttttagtatttaattcttcacggtaagaaaatgaagttctaatgaactgtttctcccaaggttttaaaattgtcaagagttattctgtttgtttaaaaaataagaaacctctttaagcaatagattttgcttgggttttcttttttaaaaacataatactgtgcaggcaaggcactgtaaaagttttaattccttccagaagaaccagtggaagaatttaaatttggcgctacgatcaaaactactgaattagtagaaataatgatgtctaaagcttaccaacaaaagaaccctcagcagaataacaaaaactttgctcaggacatttgaggtcaaattgaagacggaaaccggaaaccgttttcttgtaagcccctagaggcagatcaggtaaagcatacatagtagagggaaaggagagaatggaaataaaactcaatattatgcagatttatgccttattttttagcattttttaaggttgggtctttcaggctggttttggtttgtattagatctgtatagtttaattaactggtgatttagttttatatttaagctacaattaatcttttttctttggtgatatttatttctttgccttttttttttttaacaactttcaatcttcagatgtttcgttgaatctatttagagcttcaccatggcaatatgtatttcccttaaaacactgcaaacaaatatactaggagtgtgcccttttaatctttactagttattgtgagattgctgtgtaagctaataaacacatttgtaaatacattgtttgcaggacgaaaacttctgagttacagctcaggaaaagcctgctgaatttatgttgtaagcattacttaacacagtataaagatgaaaagacaacaaaaatatcttcatacttcctcatcccctcattggaacaaaaccttaaactgggagaaccttagtcccctctctttcctcttcctcctccacttcccacttattgtcaccttgtaatattcagagagcacttggattatggatctgaatagagaaatgcttacagataatcattagcccacataccagtaacttatacttaaagatgggatggagttgtaaagtgcttttataatacaatataattgttaaaggcaagggttgactctttgttttattttgacatggcatgtcctgaaataaatattgattcaatatggcagatgggtcatattctttatttggaagaagttgtgacttctgacatgggtgtgattgtcttcctacactgttgcatttgattctttttatgtatttttaagaaagtaaccagttatactgcttttaatattgattggtctttttatttggcttggagttcttcaaagcattgaagtgtgttcatagtccaggttttttttttaataaacacaattttgctgccaaaaatatataaataaaacacgaaagaaaacaaaaaaaaaaaaa

SEQ ID NO: 14 - protein, 653 aa, Homo sapiens
MADTLPSEFDVIVIGTGLPESIIAAACSRSGRRVLHVDSRSYYGGNWASFSFSGLLSWLKEYQENSDIVSDSPVWQDQILENEEAIALSRKDKTIQHVEVFCYASQDLHEDVEEAGALQKNHALVTSANSTEAADSAFLPTEDESLSTMSCEMLTEQTPSSDPENALEVNGAEVTGEKENHCDDKTCVPSTSAEDMSENVPIAEDTTEQPKKNRITYSQIIKEGRRFNIDLVSKLLYSRGLLIDLLIKSNVSRYAEFKNITRILAFREGRVEQVPCSRADVFNSKQLTMVEKRMLMKFLTFCMEYEKYPDEYKGYEEITFYEYLKTQKLTPNLQYIVMHSIAMTSETASSTIDGLKATKNFLHCLGRYGNTPFLFPLYGQGELPQCFCRMCAVFGGIYCLRHSVQCLVVDKESRKCKAIIDQFGQRIISEHFLVEDSYFPENMCSRVQYRQISRAVLITDRSVLKTDSDQQISILTVPAEEPGTFAVRVIELCSSTMTCMKGTYLVHLTCTSSKTAREDLESVVQKLFVPYTEMEIENEQVEKPRILWALYFNMRDSSDISRSCYNDLPSNVYVCSGPDCGLGNDNAVKQAETLFQEICPNEDFCPPPPNPEDIILDGDSLQPEASESSAIPEANSETFKESTNLGNLEESSE

SEQ ID NO: 15 - DNA, 1962 bp, Homo sapiens
atggcggatactctcccttcggagtttgatgtgatcgtaatagggacgggtttgcctgaatccatcattgcagctgcatgttcaagaagtggccggagagttctgcatgttgattcaagaagctactatggaggaaactgggccagttttagcttttcaggactattgtcctggctaaaggaataccaggaaaacagtgacattgtaagtgacagtccagtgtggcaagaccagatccttgaaaatgaagaagccattgctcttagcaggaaggacaaaactattcaacatgtggaagtattttgttatgccagtcaggatttgcatgaagatgtcgaagaagctggtgcactgcagaaaaatcatgctcttgtgacatctgcaaactccacagaagctgcagattctgccttcctgcctacggaggatgagtcattaagcactatgagctgtgaaatgctcacagaacaaactccaagcagcgatccagagaatgcgctagaagtaaatggtgctgaagtgacaggggaaaaagaaaaccattgtgatgataaaacttgtgtgccatcaacttcagcagaagacatgagtgaaaatgtgcctatagcagaagataccacagagcaaccaaagaaaaacagaattacttactcacaaattattaaagaaggcaggagatttaatattgatttagtatcaaagctgctgtattctcgaggattactaattgatcttctaatcaaatctaatgttagtcgatatgcagagtttaaaaatattaccaggattcttgcatttcgagaaggacgagtggaacaggttccgtgttccagagcagatgtctttaatagcaaacaacttactatggtagaaaagcgaatgctaatgaaatttcttacattttgtatggaatatgagaaatatcctgatgaatataaaggatatgaagagatcacattttatgaatatttaaagactcaaaaattaacccccaacctccaatatattgtcatgcattcaattgcaatgacatcagagacagccagcagcaccatagatggtctcaaagctaccaaaaactttcttcactgtcttgggcggtatggcaacactccatttttgtttcctttatatggccaaggagaactcccccagtgtttctgcaggatgtgtgctgtgtttggtggaatttattgtcttcgccattcagtacagtgccttgtagtggacaaagaatccagaaaatgtaaagcaattatagatcagtttggtcagagaataatctctgagcatttcctcgtggaggacagttactttcctgagaacatgtgctcacgtgtgcaatacaggcagatctccagggcagtgctgattacagatagatctgtcctaaaaacagattcagatcaacagatttccattttgacagtgccagcagaggaaccaggaacttttgctgttcgggtcattgagttatgttcttcaacgatgacatgcatgaaaggcacctatttggttcatttgacttgcacatcttctaaaacagcaagagaagatttagaatcagttgtgcagaaattgtttgttccatatactgaaatggagatagaaaatgaacaagtagaaaagccaagaattctgtgggctctttacttcaatatgagagattcgtcagacatcagcaggagctgttataatgatttaccatccaacgtttatgtctgctctggcccagattgtggtttaggaaatgataatgcagtcaaacaggctgaaacacttttccaggaaatctgccccaatgaagatttctgtccccctccaccaaatcctgaagacattatccttgatggagacagtttacagccagaggcttcagaatccagtgccataccagaggctaactcggagactttcaaggaaagcacaaaccttggaaacctagaggagtcctctgaataa

SEQ ID NO: 16 - DNA, 1992 bp, Homo sapiens
gatatcgaattcctgcagcccggcggcaccatggcggatactctcccttcggagtttgatgtgatcgtaatagggacgggtttgcctgaatccatcattgcagctgcatgttcaagaagtggccggagagttctgcatgttgattcaagaagctactatggaggaaactgggccagttttagcttttcaggactattgtcctggctaaaggaataccaggaaaacagtgacattgtaagtgacagtccagtgtggcaagaccagatccttgaaaatgaagaagccattgctcttagcaggaaggacaaaactattcaacatgtggaagtattttgttatgccagtcaggatttgcatgaagatgtcgaagaagctggtgcactgcagaaaaatcatgctcttgtgacatctgcaaactccacagaagctgcagattctgccttcctgcctacggaggatgagtcattaagcactatgagctgtgaaatgctcacagaacaaactccaagcagcgatccagagaatgcgctagaagtaaatggtgctgaagtgacaggggaaaaagaaaaccattgtgatgataaaacttgtgtgccatcaacttcagcagaagacatgagtgaaaatgtgcctatagcagaagataccacagagcaaccaaagaaaaacagaattacttactcacaaattattaaagaaggcaggagatttaatattgatttagtatcaaagctgctgtattctcgaggattactaattgatcttctaatcaaatctaatgttagtcgatatgcagagtttaaaaatattaccaggattcttgcatttcgagaaggacgagtggaacaggttccgtgttccagagcagatgtctttaatagcaaacaacttactatggtagaaaagcgaatgctaatgaaatttcttacattttgtatggaatatgagaaatatcctgatgaatataaaggatatgaagagatcacattttatgaatatttaaagactcaaaaattaacccccaacctccaatatattgtcatgcattcaattgcaatgacatcagagacagccagcagcaccatagatggtctcaaagctaccaaaaactttcttcactgtcttgggcggtatggcaacactccatttttgtttcctttatatggccaaggagaactcccccagtgtttctgcaggatgtgtgctgtgtttggtggaatttattgtcttcgccattcagtacagtgccttgtagtggacaaagaatccagaaaatgtaaagcaattatagatcagtttggtcagagaataatctctgagcatttcctcgtggaggacagttactttcctgagaacatgtgctcacgtgtgcaatacaggcagatctccagggcagtgctgattacagatagatctgtcctaaaaacagattcagatcaacagatttccattttgacagtgccagcagaggaaccaggaacttttgctgttcgggtcattgagttatgttcttcaacgatgacatgcatgaaaggcacctatttggttcatttgacttgcacatcttctaaaacagcaagagaagatttagaatcagttgtgcagaaattgtttgttccatatactgaaatggagatagaaaatgaacaagtagaaaagccaagaattctgtgggctctttacttcaatatgagagattcgtcagacatcagcaggagctgttataatgatttaccatccaacgtttatgtctgctctggcccagattgtggtttaggaaatgataatgcagtcaaacaggctgaaacacttttccaggaaatctgccccaatgaagatttctgtccccctccaccaaatcctgaagacattatccttgatggagacagtttacagccagaggcttcagaatccagtgccataccagaggctaactcggagactttcaaggaaagcacaaaccttggaaacctagaggagtcctctgaataa

SEQ ID NO: 17 - protein, 221 aa, Homo sapiens
MSDGDYDYLIKFLALGDSGVGKTSVLYQYTDGKFNSKFITTVGIDFREKRVVYRASGPDGATGRGQRIHLQLWDTAGQERFRSLTTAFFRDAMGFLLLFDLTNEQSFLNVRNWISQLQMHAYCENPDIVLCGNKSDLEDQRVVKEEEAIALAEKYGIPYFETSAANGTNISQAIEMLLDLIMKRMERCVDKSWIPEGVVRSNGHASTDQLSEEKEKGACGC

(19) United States
(12) Patent Application Publication
(10) Pub. No.: US 2020/0341015 A1
(43) Pub. Date: Oct. 29, 2020

(54) PRENYLATION ASSAY

(71) Applicants: NightstaRx Ltd., London (GB); Oxford University Innovation Limited, Oxford Oxfordshire (GB)

(72) Inventors: Robert Maclaren, Oxford (GB); Maria Ines Moreira Patricio, Oxford, Oxfordshire (GB); Gregory S. Robinson, Waltham, MA (US)

(21) Appl. No.: 16/757,020

(22) PCT Filed: Oct. 17, 2018
(86) PCT No.: PCT/US18/56336

§ 371 Date: Apr. 17, 2020

Related U.S. Application Data
(60) Provisional application No. 62/573,522, filed on Oct. 17, 2017.
(60) Provisional application No. 62/636,722, filed on Feb. 28, 2018.

(51) Int. Cl. G01N 33/92; C12Q 1/48; C12N 15/86

(52) U.S. Cl. CPC G01N 33/92; C12Q 1/48; C12N 2750/14143; G01N 2333/91171; G01N 2405/00; C12N 15/86

(57) ABSTRACT

A method for determining the activity of Rab escort protein 1 (REP1) comprising the steps: (a) providing a sample comprising REP1; (b) contacting the sample of step (a) with Rab6a, Rab geranylgeranyltransferase (Rab GGTase) and a lipid donor substrate; and (c) detecting the lipidated Rab6a product.

### RELATED APPLICATIONS

This application claims the benefit of provisional application U.S. Ser. No. 62/573,522, filed Oct. 17, 2017 and of provisional application U.S. Ser. No. 62/636,722, filed Feb. 28, 2018, the contents of each of which are herein incorporated by reference in their entirety.

### INCORPORATION OF SEQUENCE LISTING

The contents of the text file named “NIGH-005_001WO_SegList.txt,” which was created on Oct. 16, 2018 and is 59 KB in size, are hereby incorporated by reference in their entirety.

### FIELD OF THE DISCLOSURE

The present invention relates to an assay for use in determining the activity of Rab escort protein 1 (REP1). More specifically, the invention relates to the use of Rab6a in an assay as a substrate for prenylation, in particular wherein the REP1 has been delivered to a cell using a gene therapy vector.

### BACKGROUND OF THE DISCLOSURE

Choroideremia may be successfully treated by providing functional copies of the REP1 transgene to the affected cells of the eye. Specifically, it has been shown that adeno-associated virus (AAV) gene therapy vectors may be used to deliver a nucleotide sequence encoding functional REP1 to the eye to treat the disease. As gene therapy of choroideremia is becoming a clinical reality, there is a need for reliable and sensitive assays to determine the activity of exogenously delivered REP1, in particular to test new gene therapy vectors and as a quality control screen for clinical vector stocks. The disclosure provides a reliable and sensitive assay to determine the activity of exogenously delivered REP1.

### SUMMARY OF THE INVENTION

The present inventors have surprisingly found that, despite a prevailing understanding that Rab27a (also referred to as RAB27A) provides the most suitable prenylation assay substrate, use of Rab6a (also referred to as RAB6A) as a substrate in a prenylation reaction provides a more sensitive method for determining the activity of Rab escort protein 1 (REP1). Not only does Rab6a provide for increased sensitivity in an assay detecting prenylation, it also provides for beneficial signal-to-noise ratios, better dynamic range of signal and better consistency.

Moreover, the inventors have demonstrated that the increased sensitivity of a Rab6a-based assay may be harnessed to accurately and reliably determine the activity of REP1-encoding vectors, in particular AAV gene therapy vectors, such as those suitable for use in the clinic.

The disclosure provides a method for determining an activity of Rab escort protein 1 (REP1) comprising the steps: (a) contacting a REP1 protein with a Rab6a protein, a Rab geranylgeranyltransferase (Rab GGTase) and a lipid donor substrate to produce a lipidated Rab6a; and (b) detecting the lipidated Rab6a.

In some embodiments of the methods of determining an activity of Rab escort protein 1 (REP1) of the disclosure, a sample comprises the REP1 protein. In some embodiments, the sample comprising the REP1 protein is isolated or derived from a cell and wherein the cell is genetically engineered to express the REP1 protein. In some embodiments, the sample comprising the REP1 protein comprises a lysate of the cell.

In some embodiments of the methods of determining an activity of Rab escort protein 1 (REP1) of the disclosure, the REP1 protein is expressed from a viral vector comprising a nucleotide sequence encoding the REP1 protein. In some embodiments, the viral vector is an adeno-associated viral (AAV) vector.

In some embodiments of the methods of determining an activity of Rab escort protein 1 (REP1) of the disclosure, the Rab6a protein or the Rab GGTase is substantially pure. In some embodiments, the Rab6a protein and the Rab GGTase are substantially pure. In some embodiments, the Rab6a:Rab GGTase molar ratio is about 1:2-3. In some embodiments, the Rab6a:Rab GGTase molar ratio is 1:2-3. In some embodiments, the Rab6a:Rab GGTase molar ratio is about 1:2.5. In some embodiments, the Rab6a:Rab GGTase molar ratio is 1:2.5.

In some embodiments of the methods of determining an activity of Rab escort protein 1 (REP1) of the disclosure, the lipid donor substrate comprises geranylgeranylpyrophosphate (GGPP) or an analogue thereof. In some embodiments, the lipid donor substrate comprises biotin-geranylpyrophosphate (BGPP).

In some embodiments of the methods of determining an activity of Rab escort protein 1 (REP1) of the disclosure, detecting the lipidated Rab6a comprises an enzyme-linked immunosorbent assay (ELISA), a Western blot analysis or an autoradiography.

In some embodiments of the methods of determining an activity of Rab escort protein 1 (REP1) of the disclosure, the AAV vector comprising nucleotide sequence encoding the REP1 protein is manufactured for use in the treatment of choroideremia. In some embodiments, the lipidated Rab6a is detected and the REP-1 protein or the AAV vector comprising nucleotide sequence encoding the REP1 protein is suitable for use in the treatment of choroideremia.

In some embodiments of the methods of determining an activity of Rab escort protein 1 (REP1) of the disclosure, detecting the lipidated Rab6a further comprises quantifying an amount of the lipidated Rab6a. In some embodiments, the amount of lipidated Rab6a is an absolute amount. In some embodiments, the amount of lipidated Rab6a is a relative amount. In some embodiments, the amount of lipidated Rab6a is relative to a control amount or to a reference level.

The disclosure provides a use of a Rab6a protein for determining an activity of a Rab escort protein 1 (REP1) protein.

In some embodiments of the use of a Rab6a protein for determining an activity of a REP1 protein of the disclosure, the REP1 protein is isolated or derived from a cell and the cell is genetically-engineered to express the REP1 protein. In some embodiments, a cell comprises the REP1 protein and the cell is genetically engineered to express the REP1 protein. In some embodiments, the REP1 protein is isolated or derived from a lysate of from a cell and the cell is genetically engineered to express the REP1 protein. In some embodiments, a cell lysate comprises the REP1 protein, the cell lysate is isolated or derived from a cell, and the cell is genetically-engineered to express the REP1 protein.

In some embodiments of the use of a Rab6a protein for determining an activity of a REP1 protein of the disclosure, the REP1 protein is expressed from a viral vector comprising a nucleotide sequence encoding the REP1 protein. In some embodiments, the viral vector is an adeno-associated viral (AAV) vector. In some embodiments, the AAV vector comprising the nucleotide sequence encoding the REP1 protein is manufactured for use in the treatment of choroideremia. In some embodiments, the lipidated Rab6a is detected and the REP-1 protein or the AAV vector comprising nucleotide sequence encoding the REP1 protein is suitable for use in the treatment of choroideremia.

In some embodiments of the use of a Rab6a protein for determining an activity of a REP1 protein of the disclosure, the Rab6a protein is substantially pure.

The disclosure provides a method for determining the activity of Rab escort protein 1 (REP1) comprising the steps: (a) providing a sample comprising REP1; (b) contacting the sample of step (a) with Rab6a, Rab geranylgeranyltransferase (Rab GGTase) and a lipid donor substrate; and (c) detecting the lipidated Rab6a product.

For example, the method of the invention may be for testing gene therapy vectors suitable for the delivery of REP1 to a target cell or for quality control analysis of vector stocks (e.g. medicament stocks).

Validation of gene therapy vectors is mandatory for the safe and efficacious implementation of gene therapy in the clinic. For analysis and quality control steps, comparison with control experiments or reference levels may provide a measure of the activity of the gene therapy vector or REP1 relative to a known or accepted standard (e.g. better or worse than a known or accepted standard). This may be used to validate whether a gene therapy vector stock meets specific targets and regulations.

In one embodiment, comparison is made to a sample of REP1 or REP1-encoding AAV vector that is defined as a primary reference standard. The method of the invention may be, for example, carried out in parallel on a test sample and the primary reference standard sample. Potency, biological activity and/or behavior of the test sample may be, for example, defined relative to the primary reference standard.

Put another way, the method of the invention may, for example, be used for quality control analysis and validation of a gene therapy vector as efficacious (e.g. for the treatment of choroideremia), preferably an AAV vector particle comprising a REP1-encoding nucleotide sequence, preferably wherein an output activity or efficacy of the vector determined by the method of the invention above a threshold activity or within a specified target range (e.g. by comparison to a control experiment or reference level) indicates the vector is suitable for gene therapy purposes.

Accordingly, in another aspect, the method of the invention is for quality control analysis of a Rab escort protein 1 (REP1)-encoding gene therapy vector (preferably an AAV vector).

In another aspect, the invention provides a method for quality control analysis of a Rab escort protein 1 (REP1)-encoding gene therapy vector (preferably an AAV vector) comprising the steps: (a) transducing a cell with the vector, culturing the cell under conditions suitable for the expression of the REP1 and lysing the cells to provide a sample comprising REP1; (b) contacting the sample of step (a) with Rab6a, Rab geranylgeranyltransferase (Rab GGTase) and a lipid donor substrate; and (c) detecting the lipidated Rab6a product.

Accordingly, the method may comprise carrying out a plurality of experiments comprising steps (a) to (c) in which parameters relating to the sample comprising REP1 are varied, while other parameters (e.g. parameters relating to the Rab6a, Rab GGTase and lipid donor substrate) are kept constant. Such parameters may include, for example, the amino acid sequence of the relevant protein (e.g. REP1), the REP1-encoding nucleotide sequence comprised in a vector used to express the REP1 in a cell, the type of vector used to deliver a REP1-encoding nucleotide sequence to a cell (e.g. the type of viral vector, such as the type of adeno-associated viral (AAV) vector), the concentration of REP1 and/or the multiplicity-of-infection (MOI) of a vector used to deliver a REP1-encoding nucleotide sequence to a cell. In a preferred embodiment, the method comprises carrying out a plurality of experiments comprising steps (a) to (c) at different MOIs of a vector used to deliver a REP1-encoding nucleotide sequence to a cell (e.g. to generate a dose-response curve).

In one embodiment, the detection of the lipidated Rab6a product comprises quantifying the amount of the lipidated Rab6a product. In a preferred embodiment, the detection of the lipidated Rab6a product comprises quantifying the amount of the lipidated Rab6a product relative to a control or reference level. The quantification may be, for example, made relative to a sample of REP1 or REP1-encoding AAV vector that is defined as a primary reference standard. The method of the invention may be, for example, carried out in parallel on a test sample and the primary reference standard sample. Potency, biological activity and/or behavior of the test sample may be, for example, defined relative to the primary reference standard.

In one embodiment, the method comprises a further step of comparing the amount of lipidated Rab6a product (e.g. prenylated, such as geranylgeranylated or biotin-geranylated, Rab6a) with an amount determined from a control experiment, such as an experiment using a known or standard sample of REP1.

In another embodiment, the method comprises a further step of comparing the amount of lipidated Rab6a product (e.g. prenylated, such as geranylgeranylated or biotin-geranylated, Rab6a) with a reference level.

In one embodiment, the sample comprising REP1 is from a cell genetically engineered to express the REP1. Preferably, the sample comprising REP1 is a lysate of a cell genetically engineered to express the REP1. Preferably, a cell is transfected or transduced with a vector comprising a REP1-encoding nucleotide sequence to provide the cell genetically engineered to express the REP1. Preferably, the vector is a viral vector.

In one embodiment, the REP1 is expressed using a viral vector comprising a REP1-encoding nucleotide sequence.

In one embodiment, the viral vector is an adeno-associated viral (AAV) vector Preferably the viral vector is in the form of a viral vector particle.

The AAV vector may be of any serotype (e.g. comprise any AAV serotype genome and/or capsid protein). Preferably, the vector is capable of infecting or transducing cells of the eye.

In one embodiment, the AAV vector comprises an AAV serotype 1, 2, 3, 4, 5, 6, 7, 8, 9, 10, or 11 genome. In another embodiment, the AAV vector comprises an AAV serotype 2, 4, 5 or 8 genome. Preferably, the AAV vector comprises an AAV serotype 2 genome.

In one embodiment, the AAV vector particle comprises an AAV serotype 1, 2, 3, 4, 5, 6, 7, 8, 9, 10, or 11 capsid protein. In another embodiment, the AAV vector particle comprises an AAV serotype 2, 4, 5 or 8 capsid protein. The AAV serotype 8 capsid protein may, for example, be an AAV8/Y733F mutant capsid protein. Preferably, the AAV vector particle comprises an AAV serotype 2 capsid protein.

In one embodiment, the AAV vector particle comprises an AAV2 genome and AAV2 capsid proteins (AAV2.2); an AAV2 genome and AAV5 capsid proteins (AAV2/5); or an AAV2 genome and AAV8 capsid proteins (AAV2/8). Preferably, the AAV vector particle comprises an AAV2 genome and AAV2 capsid proteins (AAV2/2).

The AAV vector particle may be a chimeric, shuffled or capsid-modified derivative of one or more naturally occurring AAVs. In particular, the AAV vector particle may comprise capsid protein sequences from different seratypes, clades, clones or isolates of AAV within the same vector (i.e. a pseudotyped vector). Thus, in one embodiment the AAV vector is in the form of a pseudotyped AAV vector particle.

In one embodiment, the REP1 is human REP1.

In one embodiment, the REP1 comprises an amino acid sequence that has at least 70%, 80%, 85%, 90%, 91%, 92%, 93%, 94%, 95%, 96%, 97%, 98%, 99% or 100% identity to SEQ ID NO: 5, preferably wherein the amino acid sequence substantially retains the natural function of the protein represented by SEQ ID NO: 5.

In one embodiment, the REP1-encoding nucleotide sequence comprises a nucleotide sequence that has at least 70%, 80%, 85%, 90%, 91%, 92%, 93%, 94%, 95%, 96%, 97%, 98%, 99% or 100% identity to SEQ ID NO: 6 or 7, preferably wherein the protein encoded by the nucleotide sequence substantially retains the natural function of the protein represented by SEQ ID NO: 5.

In one embodiment, the REP1-encoding nucleotide sequence comprises a nucleotide sequence that encodes an amino acid sequence that has at least 70%, 80%, 85%, 90%, 91%, 97%, 93%, 94%, 95%, 96%, 97%, 98%, 99% or 100% identity to SEQ ID NO: 5, preferably wherein the amino acid sequence substantially retains the natural function of the protein represented by SEQ ID NO: 5.

In one embodiment, the Rab6a and/or Rab GGTase are substantially pure.

In one embodiment, the Rab6a comprises an amino acid sequence that has at least 70%, 80%, 85%, 90%, 91%, 92%, 93%, 94%, 95%, 96%, 97%, 98%, 99% or 100% identity to SEQ ID NO: 1, preferably wherein the amino acid sequence substantially retains the natural function of the protein represented by SEQ ID NO: 1.

In one embodiment, the Rab GGTase comprises an amino acid sequence that has at least 70%, 80%, 85%, 90%, 91%, 92%, 93%, 94%, 95%, 96%, 97%, 98%, 99% or 100% identity to SEQ ID NO: 3 or 8, preferably SEQ ID NO: 8. preferably wherein the amino acid sequence substantially retains the natural function of the protein represented by SEQ ID NO: 8; and/or an amino acid sequence that has at least 70%, 80%, 85%, 90%, 91%, 92%, 93%, 94%, 95%, 96%, 97%, 98%, 99% or 100% identity to SEQ ID NO: 4 or 9, preferably SEQ ID NO: 9, preferably wherein the amino acid sequence substantially retains the natural function of the protein represented by SEQ ID NO: 9.

In another embodiment, the Rab6a:Rab GGTase molar ratio is about 1:0.25-3, 1:0.3-2.9, 1:0.35-2.8, 1:0.4-2.7, 1:0.45-2.6 or 1:0.5-2.5, preferably about 1:0.5-2.5.

In one embodiment, the Rab6a:Rab GGTase molar ratio is about 1:2-3, 1:2.1-2.9, 1:2.2-2.8, 1:2.3-2.7 or 1:2.4-2.6, preferably about 1:2.4-2.6. In one embodiment, the Rab6a:Rab GGTase molar ratio is about 1:2, 1:2.1, 1:2.2, 1:2.3, 1:2.4, 1:2.5, 1:2.6, 1:2.7, 1:2.8, 1:2.9 or 1:3, preferably about 1:2.5.

In another embodiment, the Rab6a:Rab GGTase molar ratio is about 1:0.25-0.75, 1:0.3-0.7, 1:0.35-0.65, 1:0.4-0.6 or 1:0.45-0.55, preferably about 1:0.4-0.6. In one embodiment, the Rab6a:Rab GGTase molar ratio is about 1:0.25, 1:0.3, 1:0.35, 1:0.4, 1:0.45, 1:0.5, 1:0.55, 1:0.6, 1:0.65, 1:0.7 or 1:0.75, preferably about 1:0.5.

In one embodiment, the lipid donor substrate is geranylgeranylpyrophosphate (GGPP) or an analogue thereof. Preferably, the lipid donor substrate is labelled with a detectable marker. For example, the lipid donor substrate may be isotopically labelled (e.g. the lipid donor substrate may comprise 3H), or may comprise a fluorescent group, epitope or biotin moiety.

In a preferred embodiment, the lipid donor substrate is biotin-geranylpyrophosphate (BGPP).

In one embodiment, the lipidated Rab6a product is detected using an enzyme-linked immunosorbent assay (ELISA), Western blot analysis or autoradiography. In a preferred embodiment, the lipidated Rab6a product is detected using an ELISA. The ELISA may be, for example, a sandwich ELISA.

In a preferred embodiment, a biotin-labelled lipidated Rab6a product is detected using a detection reagent specific for biotin, for example streptavidin. Preferably, the biotin-labelled lipidated Rab6a product is detected using Western blot analysis using a detection reagent specific for biotin, for example streptavidin (e.g. a streptavidin-horseradish peroxidase conjugate). More preferably, the biotin-labelled lipidated Rab6a product is detected using an ELISA using a detection reagent specific for biotin, for example streptavidin.

In one embodiment, the method is for determining the activity of a REP1-encoding gene therapy vector for use in the treatment of choroideremia.

In another aspect, the invention provides the use of Rab6a for determining the activity of Rab escort protein 1 (REP1).

The method of determining the activity of REP1, the Rab6a, Rab GGTase, lipid donor substrate and the REP1 may be as described herein.

In another aspect, the invention provides a method for determining the efficacy of a vector comprising a Rab escort protein 1 (REP1) encoding nucleotide sequence, wherein the method comprises the steps: (a) providing a sample comprising REP1, wherein the REP1 is expressed using the vector comprising a REP1-encoding nucleotide sequence; (b) contacting the sample of step (a) with Rab6a, Rab geranylgeranyltransferase (Rab GGTase) and a lipid donor substrate; and (c) detecting the lipidated Rab6a product.

In another aspect, the invention provides the use of Rab6a for determining the efficacy of a vector comprising a Rab escort protein 1 (REP1)-encoding nucleotide sequence.

Preferably, the method and use are for determining the efficacy of a vector for use in the treatment of choroideremia.

The vector, REP1, Rab6a, Rab GGTase, lipid donor substrate, lipidated Rab6a product, method of detection and other features of the method and use may be as described herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A-D is series of photographs depicting an in vitro prenylation reaction using Rab27a as a substrate followed by Western blot (WB) analysis of incorporated biotinylated lipid donor using untransduced cells and cells transduced with either AAV-GFP or AAV-REP1 (MOI=10,000 gp/cell). The experiment involved 3 sets of lysates prepared independently. Prenylation reactions were set up using 10 μg of lysate in a total volume of 12.5 μL. Positive controls were spiked with 2 μM of fish REP1. Detection time was 2 min. (A) WB analysis of human REP1 levels in cell lysates (1:1000). Untransduced cells (#4, #9 and #12) show endogenous levels of REP1. Cells transduced with AAV-GFP (#5, #10 and #3) show endogenous levels of REP1 similar to untransduced cells. Cells transduced with AAV-REP1 (#6, #1 1 and #14) show an increase of REP1 levels compared to untransduced and AAV-GFP transduced cells. Positive controls (+ve) show endogenous REP1 levels. (B) WB analysis of β-actin as loading control (1:15000). The levels of β-actin are similar in all samples analyzed. (C) WB analysis of incorporated biotinylated lipid donor in Rab27a (1:10000). Untransduced cells and AAV-GFP transduced cells show no detectable incorporation of biotin in Rab27a. Cells transduced with AAV-REP1 show some level of biotin incorporated into Rab27a (#6, #1 1 and #14). Positive controls show the strongest band of all as a result of fish REP1 activity. (D) Semiquantification of the WB analysis in (C) using Image Studio Lite software.

FIG. 2A-D is a series of photographs depicting and in vitro prenylation reaction using Rab27a as a substrate followed by Western blot (WB) analysis of incorporated biotinylated lipid donor using untransduced cells and cells transduced with either AAV-GFP or AAV-REP1 (MOI=10,000 gp/cell). The experiment involved 2 sets of lysates prepared independently. Prenylation reactions were set up using 30 μg of lysate in a total volume of 22 μL. Positive controls were spiked with 1 μM of fish REP1. Detection time was 2 min. (A) WB analysis of human REP1 levels in cell lysates (1:1000). Untransduced cells (#9 and #12) show endogenous levels of REP1. Cells transduced with AAV-GFP (#10 and #13) show endogenous levels of REP1 similar to untransduced cells. Cells transduced with AAV-REP1 (#1 1 and #14) show an increase of REP1 levels compared to untransduced and AAV-GFP transduced cells. Positive controls (+ve) show endogenous REP1 levels. (B) WB analysis of β-actin as loading control (1:15000). The levels of β-actin are similar in all samples analyzed. (C) WB analysis of incorporated biotinylated lipid donor in Rab27a (1:10000). Untransduced cells and AAV-GFP transduced cells show no detectable incorporation of biotin in Rab27a. Cells transduced with AAV-REP1 show some level of biotin incorporated into Rab27a (#11 and #14). Positive controls show the strongest band of all as a result of fish REP1 activity. (D) Semiquantification of the WB analysis in (C) using Image Studio Lite software.

FIG. 3A-D is a series of photographs depicting an in vitro prenylation reaction using Rab6a as a substrate followed by Western blot (WB) analysis of incorporated biotinylated lipid donor using untransduced cells and cells transduced with either AAV-GFP or AAV-REP1 (MOI=10,000 gp/cell). The experiment involved 2 sets of lysates prepared independently. Prenylation reactions were set up using 20 μg of lysate in a total volume of 20 μL. Positive controls were spiked with 1 μM of fish REP1. Detection time was 2 min. (A) WB analysis of human REP1 levels in cell lysates (1:1000). Untransduced cells (#9 and #12) show endogenous levels of REP1. Cells transduced with AAV (#10 and #13) show endogenous levels of REP1 similar to untransduced cells. Cells transduced with AAV-REP1 (#1 1 and #14) show an increase of REP1 levels compared to untransduced and AAV-GFP transduced cells, Positive controls (+ve) show endogenous REP1 levels. (B) WB analysis of β-actin as loading control (1:15000). The levels of β-actin are similar in all samples analyzed. (C) WB analysis of incorporated biotinylated lipid donor in Rab6a (1:10000). Untransduced cells and AAV-GFP transduced cells show very low incorporation of biotin in Rab6a. Cells transduced with AAV-REP1 show biotin incorporated into Rab6a (#11 and #14). Positive controls show the strongest band of all as a result of fish REP1 activity. (D) Semiquantification of the WB analysis in (C) using Image Studio Lite software.

FIG. 4A-D is a series of photographs depicting an in vitro prenylation reaction using Rab6a as a substrate followed by Western blot (WB) analysis of incorporated biotinylated lipid donor using untransduced cells and cells transduced with AAV-REP1 (MOI=250, 1,000, 5,000, 10,000 and 20,000 gp/cell). Prenylation reactions were set up using 20 μg of lysate in a total volume of 15 μL. The positive control was spiked with 0.5 μM of fish REP1. Detection time was 2 min. (A) WB analysis of human REP1 levels in cell lysates (1:2500). Untransduced cells show endogenous levels of REP1. Cells transduced with AAV-REP1 show an increase of REP1 levels that directly correlates with the MOI used. The positive control (+ve) shows endogenous REP1 levels. (B) WB analysis of β-actin as loading control (1:50,000). The levels of β-actin are similar in all samples analyzed. (C) WB analysis of incorporated biotinylated lipid donor in Rab6a (1:10,000). Untransduced cells show very low of biotin incorporation in Rab6a. Cells transduced with AAV-REP1 show increasingly more biotin incorporated into Rab6a. The positive control shows the strongest band of all as a result of fish REP1 activity. (D) Semiquantification of the WB analysis in (C) using Image Studio Lite software.

FIG. 5A-D is a series of photographs depicting an in vitro prenylation reaction using Rab6a as a substrate followed by Western blot (WB) analysis of incorporated biotinylated lipid donor using untransduced cells and cells transduced with AAV-REP1 (MOI=10,000 gp/cell). Prenylation reactions were set up using 20 μg of lysate in a total volume of 15 μL. The positive control was spiked with 0.5 μM of fish REP1. Detection time was 2 min for REP1/actin and 30 seconds for biotin. (A) WB analysis of human REP1 levels in cell lysates (1:2500). Untransduced cells show endogenous level of REP1. Cells transduced with AAV-REP1 show an increase of REP1 levels. The positive control (+ve) shows endogenous REP1 levels. (B) WB analysis of β-actin as loading control (1:50,000). The levels of β-actin are similar in all samples analyzed. (C) WB analysis of incorporated biotinylated lipid donor in Rab6a (1:10,000). Untransduced cells show very low biotin incorporation in Rab6a. Cells transduced with AAV-REP1 show increased biotin incorporation into Rab6a. The positive control shows the strongest band of all as a result of fish REP1 activity. (D) Semiquantification of WB analysis in (C) using Image Studio Lite software.

FIG. 6A-D is a series of photographs depicting an in vitro prenylation reaction, followed by Western blot (WB) analysis of incorporated biotinylated lipid donor in untransduced ARPE-19 cells, and ARPE-19 cells transduced with AAV-REP1. Prenylation reactions were set up using 15 μg of lysate in a total volume of 45 μL. Positive control was spiked with 0.1 μM of fish REP1. Detection time for REP1/actin: 2 min; for biotin: 30 seconds. Experiments were carried out in parallel using the following cells: (a) Untransduced cells (#86 and #87); and (h) Cells+AAV-REP1 MOI 10,000 (#90 and #91)—R&D grade vector. (A) WB analysis of human REP1 levels in cell lysates (1:2,500). Untransduced cells show an endogenous level of REP1. Cells transduced with AAV-REP1 show an increase of REP1 levels. Positive control (+ve) shows endogenous REP1 levels. (B) WB analysis of β-actin as loading control (1:50,000). The levels of β-actin are similar in all samples analyzed. (C) WB analysis of incorporated biotinylated lipid donor in Rab6a (1:10,000). Untransduced cells show very low incorporation of biotin in Rab6a; cells transduced with AAV-REP1 show increased biotin incorporation into Rab6a. Positive control shows the strongest band of all, as a result of fish REP1 activity. (D) Semiquantification of WB analysis in (C) using Image Studio Lite software.

FIG. 7A-D is a series of photographs depicting an in vitro prenylation reaction, followed by Western blot (WB) analysis of incorporated biotinylated lipid donor in untransduced HT1080 cells, and HT1080 cells transduced with AAV-REP1. Prenylation reactions were set up using 20 μg of lysate in a total volume of 20 μL. Positive control was spiked with 0.1 μM of fish REP1. Detection time for REP1/actin: 2 min; for biotin: 30 seconds. Experiments were carried out in parallel using the following cells: (a) Untransduced cells (#56 and #57); (b) Cells+AAV-REP1 MOI 10,000 (#60 and #61)—R&D grade vector; and (c) Cells+AAV-REP1 MOI 10,000 (#64 and #65)—clinical grade vector. (A) WB analysis of human REP1 levels in cell lysates (1:2,500). Untransduced cells show endogenous levels of REP1. Cells transduced with AAV-REP1 show an increase of REP1 levels. Positive control (+ve) shows endogenous REP1 levels. (B) WB analysis of β-actin as loading control (1:50,000). The levels of β-actin are similar in all samples analyzed. (C) WB analysis of incorporated biotinylated lipid donor in Rab6a (1:10,000). Untransduced cells show baseline levels of biotin incorporation in Rab6a; cells transduced with AAV-REP1 show increased biotin incorporation into Rab6a. (D) Semiquantification of WB analysis in (C) using Image Studio Lite software.

FIG. 8A-E is a series of photographs and graphs depicting an in vitro prenylation reaction, followed by Western blot (WB) analysis of incorporated biotinylated lipid donor in untransduced cells, and cells transduced with AAV-REP1 (MOI=250, 1,000, 5,000, 10,000 and 20,000 gp/cell) comparing Rab6a and Rab27a substrates. Prenylation reactions were set up using 20 μg of lysate in a total volume of 15 μL, and 2 different substrates: Rab27a (left-hand lanes and plots; in red) and Rab6a (right-hand lanes and plots; in blue). Positive controls, one for each substrate, were spiked with 0.1 μM of fish REP1. Detection time: 2 min. (A) WB analysis of human REP1 levels in cell lysates (1:2,500). Untransduced cells show endogenous level of REP1. Cells transduced with AAV-REP1 show an increase of REP1 levels that directly correlates with the MOI used. Positive control (+ve) shows endogenous REP1 levels. (B) WB analysis of β-actin as loading control (1:50,000). The levels of β-actin are similar in all samples analyzed. (C) WB analysis of incorporated biotinylated lipid donor in Rab27a and Rab6a. Untransduced cells show very low incorporation of biotin into the Rab protein, which increases as the MOI increases. Positive controls show strong biotin incorporation, as a result of fish REP1 activity. (D) Semiquantification of WB analysis in (C) using Image Studio Lite software. Data was plotted using Prism software as shown in the four plots in (E). (E) Band density values from biotinylated substrates across MOIs used (upper two plots) and ratio between biotinylated substrates and REP1 across MOIs used (lower two plots).

FIG. 9A-D is a series of tables and photographs depicting an in vitro prenylation reaction, followed by Western blot (WB) analysis of incorporated biotinylated lipid donor in untransduced cells comparing different prenylation reaction conditions. Prenylation reactions were set up using 20 μg of lysate in a total volume of 15 μL, and 2 different substrates: Rab27a (in red) and Rab6a (in blue). Positive controls, one for each substrate, were spiked with recombinant human REP1. Detection time: 2 min. (A) WB analysis of incorporated biotinylated lipid donor in Rab27a and Rab6a. Level of biotin incorporation is directly proportional to the amount of total protein in the reaction. Positive controls show strong biotin incorporation, as a result of fish REP1 activity. (B) WB analysis of β-actin as loading control (1:50,000). The levels of β-actin match the amount of total cell lysates used in the reaction, and are similar between samples. (C) WB analysis of human REP1 levels in cell lysates (1:2,500). Untransduced cells show endogenous level of REP1. Positive control (+ve) shows higher density of REP1. (D) Semiquantification of WB analysis in (A) using Image Studio Lite software. Data was plotted using Prism software. Values highlighted are for those conditions where a higher difference between substrates was detected.

FIG. 10 is a graph depicting a comparison between Rab27a and Rab6a as substrates for prenylation in AAV-REP1 transduced cells.

FIG. 11A-B is a table, photograph, and graph showing that both Rab27a and Rab6a are subject to prenylation by endogenous REP1 from a 293 cell lysate. A) Summary table of experimental conditions (#) used in prenylation reactions in vitro regarding the amount of cell lysate (I: 2.5 μg; 5 μg; 10 μg; 20 μg), concentration of GGT-II (II: 0.5 μM; 1 μM; 2 μM) and concentration of Rab substrate (Rab27a or Rab6a) (III: 0.16 μM; 0.8 μM; 4 μM), Positive control (#17; +): cell lysate spiked with recombinant human REP1. B) Protein expression (human REP1 and β-actin) and biotin incorporation detected in prenylation reaction products following SDS-PAGE and western blot analysis. The densitometry analysis of the biotinylated Rab substrate bands is depicted in a bar graph (conditions 1-16).

FIG. 12A-D is a series of photographs and graphs showing that Rab6a is more fit than Rab27a to assess the potency of human REP1 following AAV2 transduction of 293 cells. A) 293 cells were transduced with increasing MOI of AAV2-REP1 (100; 500; 1,000; 5,000; 10,000; 20,000 and 50,000). Protein expression (human REP1 and β-actin) and biotin incorporation were detected in prenylation reaction products (20 μg) following SDS-PAGE and western blot analysis (representative image of 3 replicates). B) Nonlinear regression plot of normalised REP1 (corrected for the corresponding actin levels) per log (MOI) of AAV2-REP1. Data was analysed using a sigmoidal four-parameter fit (95% CI; constrains: bottom>0; hill slope=1). Symbols are mean of 6 replicates±SEM. C) Plot of the band density values obtained for biotinylated Rab27a and Rab6a following transduction of 293 cells with AAV2-REP1 (n=3) and corrected for endogenous levels (MOI=0). Rab6a showed significantly higher values than Rab27a. at MOI 10,000, 20,000 and 50,000 (two-way ANOVA with Bonferroni's multiple comparison test; **p=0.0042; ****p<0.0001). D) Densitometry values of biotin incorporation per normalised REP1 were plotted for both Rab27a and Rab6a and analysed by linear regression (Rab27a, Y=6.335*X−0.6392; Rab6a, Y=12.6*X+0.9576).

FIG. 13A-B is a series of photographs showing Rab6a validation as a substrate for in vitro prenylation by other cell lines. Protein expression (human REP1 and β-actin) and biotin incorporation were detected in prenylation reaction products following cell transduction, SDS-PAGE and western blot analysis (two replicates in one experiment). HT-1080 cells (A) and ARPE-19 cells (B) were transduced with rAAV2/2-REP1 (MOI 1,000; 10,000 and 30,000 gc/cell) and prenylation reactions prepared with 20 μg and 10 μg of total protein, respectively. Positive controls (+rREP1) were prepared using untransduced cell lysate spiked with a recombinant fish REP1 protein (25 nM for HT-1080; 11 nM for ARPE-19).

FIG. 14A-D is a table (A), 6 photographs (B), 3 graphs (C) and a table (D) showing that both RAB27A and RAB6A are subject to prenylation by endogenous REP1 from a 293 cell lysate. A) Summary table of experimental conditions (#1-#8) used in prenylation reactions in vitro regarding the amount of total cell lysate (2.5; 5; 10; 20 μg), concentration of GGT-II (0.5; 1; 2 μM) and concentration of Rab substrate (RAB27A or RAB6A) (0.16; 0.8; 4 μM). Positive control (+ve): cell lysate spiked with recombinant fish REP1 (25 nM). B) Protein expression (human REP1 and β-actin) and biotin incorporation detected in prenylation reaction products following SDS-PAGE and western blot analysis (representative of 3 independent experiments). C) Plots for condition sets assessing biotin incorporation in both RAB27A and RAB6A when different amounts of total cell lysate, concentration of GGT-II or concentration of Rab substrate were used (n=3). D) Summary table of statistical analysis performed in the data sets in C). Two-way ANOVA tests were run independently for each condition (total cell lysate, concentration of GGT-II or concentration of Rab substrate) with ‘condition’ and ‘substrate’ as factors. The p values and the significance of each test, as well the Bonferroni's multiple comparison test for comparison of RAB27A with RAB6A, are given in detail.

FIG. 15A-D is 3 photographs (A) and 3 graphs (B-D) showing that RAB6A is more sensitive than RAB27A to assess the biological activity of human REP1 following rAAV2/2 transduction of 293 cells. A) 293 cells were transduced with increasing MOI of rAAV2/2-REP1 (100; 300; 1,000; 3,000; 10,000; 30,000; 100,000 and 300,000). Protein expression (human REP1 and β-actin) and biotin incorporation were detected in prenylation reaction products (20 μg) following SDS-PAGE and western blot analysis (representative image of 3 independent experiments). B) Nonlinear regression plot of normalized REP1 (corrected for the corresponding actin levels) per rAAV2/2-REP1 (log gc/cell). Data was analyzed using a sigmoidal four-parameter fit (95% confidence interval; R²=0.8625). Symbols are mean of 6 replicates±SEM. C) Nonlinear regression plots of biotin incorporation per MOI of rAAV2-REP1 (log gc/cell). Data was analyzed using a sigmoidal four-parameter fit (95% confidence interval; R²=0.8873 for RAB6A; R²=0.8772 for RAB27A). Symbols are mean of 3 replicates±SEM, RAB6A showed statistically significant higher incorporation of biotin than RAB27A at MOI 10,000 (**, p=0,0097), 30,000 (***p=0.0002) and 100,000 and 300,000 (****, p<0.0001) (two-way ANOVA with Bonferroni's multiple comparison test). D) Linear regression plots of biotin incorporation in substrate, corrected for the untransduced control, against the normalized overexpressed REP1 for RAB6A (R²=0.8959, Y=18.82*X+0.4803) and RAB27A (R²=0.533, Y=6.569*X+0.9042).

FIG. 16A-B are a graph (A) and viable of rhREP1 calibration standards (B) showing an enzyme-linked immunosorbent assay (ELISA) to detect REP1. Plates were coated with Rabbit anti-CHM polyclonal antibody (Sigma HPA003231) at 2 μg/mL and 100 μL per well. The block/wash was done with Superblock from Thermo Fisher Scientific. Calibration standards were with rhREP1 (NAC) at 0.5-100 ng/mL in prenylation buffer without dithiothreitol (DTT). Detection was with biotinylated mouse monoclonal 2F1 (Merck) at 0.5 μg/mL. Biotinylation was performed using a Miltenyi kit. Samples of transduced and non-transduced cell lysates were diluted 1:100 or 1:1000 with lysis buffer without DTT.

FIG. 17 is a table showing the results of a REP1 potency assay using an ELISA to detect REP1. Cells were transduced with the REP1 vector ENG1014A at a multiplicity of infection (MOI) of 10,000, lysed and REP1 was detected using ELISA. Non-trans=non transduced control, Trans=transduced cells. Samples were diluted 1:100.

FIG. 18A-C are a graph (A) and a pair of tables (B and C) showing an exemplary rAAV2-REP1 potency assay REP1 ELISA. (A) Shows concentration (x-axis) versus raw data (optical density, y-axis). (B) is a table of rhREP1 calibration standards. (C) is a table showing a rhREP1 precision profile (n=10).

FIG. 19A-B are a table (A) and diagram (B) showing prenylation principles and assays.

FIG. 20 is a table showing assessments by in vitro prenylation assays in gene therapy.

FIG. 21A-C are a pair of plots (A, C), and a diagram (B) showing a Rab hierarchy according to prenylation rate.

FIG. 22 is a diagram depicting the detection of a pool of unprenylated Rabs (background) and co-staining with Rab27a in an unprenylated pool. WT cells are depicted on the left, CHM cells on right. In the wild type cells, unprenylated Rabs are detected with biotin. The signal is expected to be low. Detection of Rab27a in the unprenylated pool is also expected to be low. In CHM cells, the detection of unprenylated Rabs and Rab27a in the unprenylated pool are expected to generate high signal.

FIG. 23A-C are a photograph of a Western Blot (A) and a pair of graphs (B, C) showing the quantification of band intensity for unprenylated Rabs. In (A) unprenylated Rabs are in green, and Rab27A is in red, WT=wild type samples (n=10), CHM=choroideremia samples (n=12). In (B) the ration of unprenylated Rabs to actin in WT and CHM samples was compared using an unpaired t-test (p=0.0362). In (C), the ratio of unprenylated Rab27a to actin in WT and CHM samples was compared using an unpaired t-test (p=0.0044).

FIG. 24 is a table showing assessments by in vitro prenylation assays.

FIG. 25 is a series of 3 photographs of Western blots showing prenylation activity in rAAV2.REP1 in a test of a 12-well plate for a functional assay. Increasing MOI of the AAV2.REP1.ENG1014-A vector are used. Left box: cells were lysed in 40 μL of buffer. Right box: cells were lysed in 50 μL of buffer. From top to bottom are shown hREP1 (83 KDa), Actin (42 KDa) and biotinylated Rab6a (24 KDa). Lanes in each box, from left to right, are 0 MOI, 300 MOI, 1,000 MOI, 3,000 MOI, 10,000 MOI, 30,000 MOI and 0 MOI+fish REP1 protein. Protein sizes are indicated from top to bottom, at left, as 100, 75, 48, 35 and 25 KDa.

FIG. 26A-C are three plots depicting prenylation activity in rAAV2.REP1 in a test of a 12-well plate for a functional assay. 50 μL cell lysate generated data consistent with previous findings. The test used 15 μg protein per reaction. (A) Normalized REP1 (a.u. REP1/a.u. Actin) is shown on the y axis, MOI as log gc/cell rAAV2/2-REP1 on the x-axis. Open circles indicate REP1 from cells lysed in 400 μL of buffer (R²=0.9845), black circles indicate REP1 from cells lysed in 50 μL of buffer (R²=0.999). (B) Biotin incorporation in substrate (a.u.) is indicated on the y-axis, MOI as log gc/cell rAAV2/2-REP1 on the x-axis. Open circles indicate REP1 from cells lysed in 40 μL of buffer (R²=0.9997), black circles indicate REP1 from cells lysed in 50 μL of buffer (R²=0.9992). (C) Biotin incorporation in substrate (a.u.) corrected for untransduced control is indicated on the y-axis, normalized overexpressed REP1 (a.u. REP1/a.u. actin) is depicted on the x-axis. x's indicate Rab6a from cells lysed in 40 μL of buffer (R²=0.8805, Y=16.2*X−4.066), open circles indicate Rab6a from cells lysed in 50 μL of buffer (R²=0.9957, Y=16.99*X−2011). a.u.=absorbance unit.

FIG. 27 is a graph showing AAV titer as determined by PCR. On the X axis are samples at an initial titer of 1×10¹² Dnase resistant particles (DRP)/mL, 1×10¹¹ DRP/mL and 1×10¹¹ DRP/mL in balanced saline solution (BSS). On the Y axis, is shown titer measured after samples were treated as described to the right of the graph.

FIG. 28 is a series of 3 photographs of Western blots showing the prenylation activity of rAAV2.REP-1 in a compatibility study using AAV2.REP1.ENG1014-A vector at a high dose of 1×10¹² DRP/mL and an MOI of 10,000. From top to bottom are shown: hREP1 (83 KDa), Actin (42 KDa) and biotinylated Rab6a (24 KDa). Protein sizes are indicated at left, from top to bottom, as 180, 135, 100, 75, 63, 48, 35, 25, 20, 17 and 11 KDa. Samples, from left to right, in triplicate, are: untransduced control, cells transduced with baseline vector, with vector held 6 hours at 4° C., with vector held 6 hours at 4° C. and injected after 180 minutes, with vector held 6 hours at 4° C. and 180 minutes in a syringe, and fish REP1 as a positive control (single sample).

FIG. 29 is a series of 3 photographs of Western blots showing the prenylation activity of rAAV2.REP-1 in a compatibility study using AAV2.REP1.ENG1014-A vector at a low dose of 1×10¹¹ DRP/mL, and an MOI of 10,000. From top to bottom are shown: hREP1 (83 KDa), Actin (42 KDa) and biotinylated Rab6a (24 KDa). Protein sizes are indicated at left, from top to bottom, as 180, 135, 100, 75, 63, 48, 35, 25, 20, 17 and 11 KDa. Samples, from left to right, in triplicate, are: untransduced control, cells transduced with baseline vector, with vector held 6 hours at 4° C., with vector held 6 hours at 4° C. and injected after 180 minutes, with vector held 6 hours at 4° C. and 180 minutes in a syringe, and fish REP1 as a positive control (single sample).

FIG. 30A-B are a pair of plots showing semi quantification of Western blots of prenylation activity of rAAV2.REP-1 in a compatibility study using AAV2.REP1.ENG1014-A vector. (A) Shows normalized REP1. Band density values (a.u.) are on the y-axis and AAV2-REP1 at a high dose of 1×10¹² DRP/mL and a low dose of 1×10¹¹ DRP/mL are on the x-axis. (B) Shows normalized biotinylated Rab6a. Band density values (a.u.) are on the y-axis and AAV2-REP1 at a high dose of 1×10¹² DRP/mL and a low dose of 1×10¹¹ DRP/mL are on the x-axis. In (A) and (B), bars for each dose, from left to right, indicate untransduced cells, cells transduced with baseline vector, with vector held 6 hours at 4° C., +6 hours at 4° C. and injected after 180 minutes at 20° C., with vector 6 hours at 4° C. and 180 minutes in a syringe at 20° C.

### DETAILED DESCRIPTION

Choroideremia is a rare disease which leads to degeneration of the choroid, retinal pigment epithelium and photoreceptors of the eye. Afflicted males typically exhibit nightblindness during teenage years, progressive loss of peripheral vision during the 20's and 30's and complete blindness in the 40's. Female carriers may maintain a good vision throughout life, but may have mild symptoms, most notably nightblindness, but may occasionally have a more severe phenotype.

Choroideremia is caused by mutations in the CHM gene, which encodes for Rab escort protein 1 (REP1). Rab escort protein 2 (REP2), which is 75% homologous to REP1, compensates for any REP1 deficiency in most cells of the body. However, REP2 is unable to compensate for REP1 deficiency in the eye. This leads to insufficient Rab escort protein activity to maintain normal prenylation of target Rab GTPases and gives rise to cellular dysfunction and ultimately cell death.

Choroideremia may be successfully treated by providing functional copies of the REP1 transgene to the affected cells of the eye (Maclaren, R. E. et al. (2014) Lancet 383: 1 129-37). Specifically, it has been shown that adeno-associated virus (AAV) gene therapy vectors may be used to deliver a nucleotide sequence encoding functional REP1 to the eye to treat the disease. As gene therapy of choroideremia is becoming a clinical reality, there is a need for reliable and sensitive assays to determine the activity of exogenously delivered REP1, in particular to test new gene therapy vectors and as a quality control screen for clinical vector stocks.

Existing methods for assaying REP1 use Rab27a as a prenylation substrate (Tolmachova, T. et al. (2012) J. Gene Med. 14: 158-168; Tolmachova, T. et al. (2013) J. Mol. Med. 91 825-837; Vasireddy, V. et al. (2013) PLoS ONE 8: e61396; and Black, A. et al. (2014) J. Gene Med. 16: 122-130). This has likely followed from numerous implications of Rab27a in the pathogenesis of choroideremia. For example, it has been shown that Rab27a is present unprenylated in choroideremia cells while other Rabs are properly prenylated (Seabra, M. C. et al. (1995) J. Biol. Chem. 270: 24420-24427). Furthermore, Rab27a is expressed at high levels in the retinal pigment epithelium and choriocapillaries, the two sites of earliest degeneration in choroideremia.

However, assays relying on the prenylation of Rab27a give rise to very weak signals. As a result, the sensitivity of these assays is low and they may not be suitable for reliable screening of clinical gene therapy vectors. Accordingly, a significant need exists for more reliable and sensitive assays which can be used to determine REP1 activity and test gene therapy vectors.

Choroideremia (CHM) is a rare, X-linked recessive retinal dystrophy caused by mutations in the CHM gene, which encodes for Rab escort protein 1 (REP1). Choroideremia leads to degeneration of the retinal pigment epithelium (RPE) and the photoreceptors of the eye. CHM is ubiquitously expressed in human cells and encodes Rab escort protein 1 (REP1). REP1 involved in the C-terminus posttranscriptional modification of Rab GTPases, the largest family within the Ras-like GTPase superfamily. This modification, known as prenylation, is catalyzed by the Rab geranylgeranyl transferase (RGGT or GGT-II) and involves the covalent attachment of one or more C20 (geranylgeranyl) isoprenoid groups to a cysteine residue within a ‘prenylation motif’. REP1 assists by either presenting the unprenylated Rabs to the GGT-II and/or escorting the prenylated Rabs to their destination membrane where they play a role in vesicle trafficking.

The choroideremia-like gene (CHML) encodes for Rab escort protein 2 (REP2). REP2 shares 95% of its amino acid sequence with REP1, and studies have shown that REP2 can compensate for REP1 deficiency in most cells of the body. However, REP2 is unable to fully compensate for REP1 deficiency in the eye. In choroideremia patients, the prenylation of Rab GTPases in the eye is affected, which causes cellular dysfunction and ultimately cell death.

REP1 plays a role in intracellular trafficking through the prenylation of Rab GTPases, a reaction that can be reproduced in vitro. Adeno-associated virus (AAV) gene replacement therapy is a treatment for choroideremia. Choroideremia may be treated by providing functional copies of the CHM gene to the affected cells of the eye. Specifically, a recombinant adeno-associated virus (rAAV) vector encoding CHM can be delivered subretinally. There is therefore a need for an assay to assess the biological activity of the vectors for the treatment of choroideremia. For example, there is a need for reliable and sensitive in vitro assays to determine the biological activity of rAAV2/2-REP1. A prenylation reaction can be reproduced in vitro to test for REP1 biological activity. One substrate for a prenylation assay following viral transduction is Rab27a. The Rab27a protein was first identified in the cytosolic fraction of CHM lymphoblasts in 1995. Another substrate for a prenylation assay in vitro is another Rab protein, RAB6A. The response of these two Rab proteins, Rab27A and RAB6A, to the incorporation of a biotinylated lipid donor in a prenylation reaction can be assayed in vitro and used to develop robust and sensitive assays for assessing the biological activity of AAV vectors for choroideremia.

Various preferred features and embodiments of the present invention will now be described by way of non-limiting examples.

The practice of the present invention will employ, unless otherwise indicated, conventional techniques of chemistry, biochemistry, molecular biology, microbiology and immunology, which are within the capabilities of a person of ordinary skill in the art. Such techniques are explained in the literature. See, for example, Sambrook, J., Fritsch, E. F. and Maniatis, T. (1989) Molecular Cloning: A Laboratory Manual, 2nd Edition, Cold Spring Harbor Laboratory Press; Ausubel, F. M. et al. (1995 and periodic supplements) Current Protocols in Molecular Biology, Ch. 9, 13 and 16, John Wiley & Sons; Roe, B., Crabtree, J. and Kahn, A. (1996) DNA Isolation and Sequencing: Essential Techniques, John Wiley & Sons; Polak, J. M. and McGee, J. O'D. (990) In Situ Hybridization: Principles and Practice, Oxford University Press; Gait, M. J. (1984) Oligonucleotide Synthesis: A Practical Approach, IRL Press; and Lilley, D. M. and Dahlberg, J. E. (1992) Methods in Enzymology: DNA Structures Part A: Synthesis and Physical Analysis of DNA, Academic Press. Each of these general texts is herein incorporated by reference.

### Prenylation

Previous methods for the detection of small GTPases in vitro used radiolabelled-prenyl donors. Radiolabelling can be replaced by either a fluorophore or a biotin group. Both approaches involve the use of a cultured cell lysate as REP1 is ubiquitously expressed in all cells and tissues. Protein incorporation of biotin-containing isoprenoids (biotin-labelled geranyl pyrophosphate, B-GPP) can be used to detect prenylated proteins due to their superior sensitivity relatively to fluorescence-based methods.

Lipidation of proteins by the addition of isoprenoid moieties is a post-translational modification that affects up to 2% of the mammalian proteome. Such lipidation enables reversible association of the target proteins with cell membranes and can also modulate protein-protein interactions.

Preferably, the lipidation referred to herein is prenylation, such that the lipid donor substrate and lipidated Rab6a product are a prenyl donor substrate and prenylated Rab6a product, respectively.

Prenylation is a specific type of post-translational modification in which a geranylgeranyl or farnesyl moiety (or analogue of either) is attached to one or two C-terminal cysteine residues of a protein via a thioether linkage.

Preferably, the prenylation is the addition of a geranylgeranyl moiety or an analogue thereof (e.g. biotin-geranyl moiety) to a target protein (e.g. Rab6a).

A geranylgeranyl moiety attached to a protein (the protein is depicted schematically by the shaded circle) is:

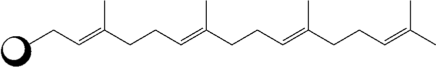 [SMILES: CC/C=C(\C)CC/C=C(\C)CC/C=C(\C)CCC=C(C)C]

A farnesyl moiety attached to a protein (the protein is depicted schematically by the shaded circle) is:

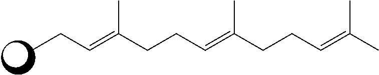 [SMILES: CC/C=C(\C)CC/C=C(\C)CCC=C(C)C]

The term “analogue” is used herein in relation to the lipid (e.g. geranylgeranyl or farnesyl) moiety or lipid donor substrate to refer to a compound which has been modified to comprise a functional group suitable for a particular purpose, such as detection. The analogue is able to be added to a substrate protein by the prenylation machinery (i.e. REP1 and Rab GGTase) in a manner substantially unhindered (for the purposes of the activity assays of the invention) by the modification.

Accordingly, analogues of the above moieties include those which have been artificially created for particular purposes (e.g. labelled moieties which are suitable for detection in an assay). In particular, Nguyen et al. (Nguyen, U. T. et al. (2009) Nat. Chem. Biol. 5: 227-235) developed the following biotin-geranyl moiety that can be detected in in vitro protein prenylation reactions (the biotin-geranyl moiety is shown attached to a protein, which is depicted schematically by the shaded circle):

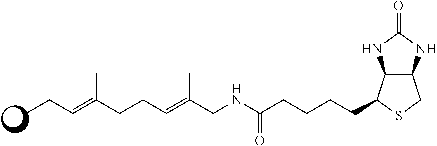 [SMILES: CC/C=C(\C)CC/C=C(\C)CNC(=O)CCCC[C@@H]1SC[C@@H]2NC(=O)N[C@@H]21]

### Rab6a

Rab6a (Ras-related protein Rab-6A) is a member of the mammalian Rab GTPase family, which is itself the largest of the Ras-like super-family of GTPases.

Rab GTPases (also known as Rab proteins) are peripheral membrane proteins and are involved in the regulation of membrane trafficking, including vesicle formation, vesicle movement along actin and tubulin networks, and membrane fusion. The main function of Rab6a is understood to be the regulation of protein transport from the Golgi complex to the endoplasmic reticulum.

Rab GTPases are typically anchored to a cell membrane via prenyl groups (in particular, geranylgeranyl groups) which are covalently bound to two C-terminal cysteine residues.

Rab GTPases exhibit two conformations: an inactive, GDP-bound form; and an active, GTP-bound form. Conversion from the GDP- to the GTP-bound forms is catalyzed by a GDP/GTP exchange factor (GEF), which thereby activates the Rab GTPase. Conversely, GTP hydrolysis by Rab GTPases can be enhanced by a GTPase-activating protein (GAP), which thereby leads to Rab inactivation.

In one embodiment, the Rab6a is human Rab6a.

An example amino acid sequence of Rab6a is the sequence deposited under NCBI Accession No. NP_942599.1 (SEQ ID NO: 1).

An example amino acid sequence of Rab6a is:

(SEQ ID NO: 1)
MSTGGDFGNPLRKFKLVFLGEQSVGKTSLITRFMYDSFDNTYQATIGIDF
LSKTMYLEDRTVRLQLWDTAGQERFRSLIPSYIRDSTVAVVVYDITNVNS
FQQTTKWIDDVRTERGSDVIIMLVGNKTDLADKRQVSIEEGERKAKELNV
MFIETSAKAGYNVKQLFRRVAAALPGMESTQDRSREDMIDIKLEKPQEQP
VSEGGCSC.

An example nucleotide sequence encoding Rab6a is the sequence deposited under NCBI Accession No. NM_198896.1 (SEQ ID NO: 13).

An example nucleotide sequence encoding Rab6a is:

(SEQ ID NO: 2)
ATGTCCACGGGCGGAGACTTCGGGAATCCGCTGAGGAAATTCAAGCTGGT
GTTCCTGGGGGAGCAAAGCGTTGGAAAGACATCTTTGATCACCAGATTCA
TGTATGACAGTTTTGACAACACCTATCAGGCAACAATTGGCATTGACTTT
TTATCAAAAACTATGTACTTGGAGGATCGAACAGTACGATTGCAATTATG
GGACACAGCAGGTCAAGAGCGGTTCAGGAGCTTGATTCCTAGCTACATTC
GTGACTCCACTGTGGCAGTTGTTGTTTATGATATCACAAATGTTAACTCA
TTCCAGCAAACTACAAAGTGGATTGATGATGTCAGAACAGAAAGAGGAAG
TGATGTTATCATCATGCTAGTAGGAAATAAAACAGATCTTGCTGACAAGA
GGCAAGTGTCAATTGAGGAGGGAGAGAGGAAAGCCAAAGAGCTGAATGTT
ATGTTTATTGAAACTAGTGCAAAAGCTGGATACAATGTAAAGCAGCTCTT
TCGACGTGTAGCAGCAGCTTTGCCGGGAATGGAAAGCACACAGGACAGAA
GCAGAGAAGATATGATTGACATAAAACTGGAAAAGCCTCAGGAGCAACCA
GTCAGTGAAGGAGGCTGTTCCTGCTAA

A further example nucleotide sequence encoding Rab6a is:

| (SEQ ID NO: 13) | |
|---|---|
| gcacgcacgc acgcacgcca gcggccggcg gggccgcagg ctcgcgcccg gcctcgcccc | 60 |
| gcgccgctcc agaggctcgc gcactcagca ggttgggctg cggcggcggc ggcagctgtg | 120 |
| gaagctcagg cgctgcgcgt gagaggtccc agatacgtct gcggttccgg ctccgccacc | 180 |
| ctcagcttct cttccccagg tctgggagcc gagtgcggaa ggagggaacg gccctagctt | 240 |
| tgggaagcca gaggacaccc ctggctcctg ccgacaccgc cctccttccc ttcccagccg | 300 |
| cgggcctcgc tcggtgctag gctactctgc cgggaggcgg cggcggctgc cagtctgtgg | 360 |
| agagtcctgc tgccctccag ccgggctcct ccaccgggcc ttgcaggggc cgagagagct | 420 |
| cggtgcccgc ccttccgctc gcctttttcg tcagctggct ggagcagcat cggtccggga | 480 |
| ggtctctagg ctgaggcggc ggccgctcct ctagttccac aatgtccacg ggcggagact | 540 |
| tcgggaatcc gctgaggaaa ttcaagctgg tgttcctggg ggagcaaagc gttggaaaga | 600 |
| catctttgat caccagattc atgtatgaca gttttgacaa cacctatcag gcaacaattg | 660 |
| gcattgactt tttatcaaaa actatgtact tggaggatcg aacagtacga ttgcaattat | 720 |
| gggacacagc aggtcaagag cggttcagga gcttgattcc tagctacatt cgtgactcca | 780 |
| ctgtggcagt tgttgtttat gatatcacaa atgttaactc attccagcaa actacaaagt | 840 |
| ggattgatga tgtcagaaca gaaagaggaa gtgatgttat catcatgcta gtaggaaata | 900 |
| aaacagatct tgctgacaag aggcaagtgt caattgagga gggagagagg aaagccaaag | 960 |
| agctgaatgt tatgtttatt gaaactagtg caaaagctgg atacaatgta aagcagctct | 1020 |
| ttcgacgtgt agcagcagct ttgccgggaa tggaaagcac acaggacaga agcagagaag | 1080 |
| atatgattga cataaaactg gaaaagcctc aggagcaacc agtcagtgaa ggaggctgtt | 1140 |
| cctgctaatc tcccatgtca tcttcaacct tcttcagaag ctcactgctt tggccccctt | 1200 |
| actctttcat tgactgcagt gtgaatattg gcttgaacct tttcccttca gtaataacgt | 1260 |
| attgcaattc atcattgctg cctgtctcgt ggagatgatc tattagcttc acaagcacaa | 1320 |
| caaaagtcag tgtcttcatt atttatattt tacaaaaagc caaaatattt cagcatattc | 1380 |
| cagtgataac tttaaaaatt agatacattt tcttaacatt tttttctttt ttaatgttat | 1440 |
| gataatgtac ttcaaaatga tggaaatctc aacagtatga gtatggcttg gttaacgagc | 1500 |
| ggtatgttca cagcctactt tatctctcct tgcttttctc acctctcact tacccccatt | 1560 |
| ccctattacc ctattcttac ctagcctccc ccgacttcct caaaacaaac aagagatggc | 1620 |
| aaagcagcag ttctaccaag cccattggaa ttatccttta attttacaga taccacttgc | 1680 |
| tgtaggctac ggaccaagat gtccaaaatt attcttgagc actgatataa attacggtct | 1740 |
| tctttgaggt caaaattcag ccatcatggt aggcagtgct tgaatgagaa aaggctcctg | 1800 |
| gtgcatcttc aaaatgagtc ctaaagaaca tactgagtac ttagaagtag aagaacataa | 1860 |
| gatgtatttc tgactaaaac aaatggctct ttcacatgtg ctttattaga ctctgggaga | 1920 |
| gaaaattaac caagtgcttc agaacaggtt tttagtattt aattcttcac ggtaagaaaa | 1980 |
| tgaagttcta atgaactgtt tctcccaagg ttttaaaatt gtcaagagtt attctgtttg | 2040 |
| tttaaaaaat aagaaacctc tttaagcaat agattttgct tgggttttct tttttaaaaa | 2100 |
| cataatactg tgcaggcaag gcactgtaaa agttttaatt ccttccagaa gaaccagtgg | 2160 |
| aagaatttaa atttggcgct acgatcaaaa ctactgaatt agtagaaata atgatgtcta | 2220 |
| aagcttacca acaaaagaac cctcagcaga ataacaaaaa ctttgctcag gacatttgag | 2280 |
| gtcaaattga agacggaaac cggaaaccgt tttcttgtaa gcccctagag gcagatcagg | 2340 |
| taaagcatac atagtagagg gaaaggagag aatggaaata aaactcaata ttatgcagat | 2400 |
| ttatgcctta ttttttagca ttttttaacm ttgggtcttt caggctggtt ttggtttgta | 2460 |
| ttagatctgt atagtttaat taactggtga tttagtttta tatttaagct acaattaatc | 2520 |
| ttttttcttt ggtgatattt atttctttgc cttttttttt tttaacaact ttcaatcttc | 2580 |
| agatgtttcg ttgaatctat ttagagcttc accatggcaa tatgtatttc ccttaaaaca | 2640 |
| ctgcaaacaa atatactagg agtgtgccct tttaatcttt actagttatt gtgagattgc | 2700 |
| tgtgtaagct aataaacaca tttgtaaata cattgtttgc aggacgaaaa cttctgagtt | 2760 |
| acagctcagg aaaagcctgc tgaatttatg ttgtaagcat tacttaacac agtataaaga | 2820 |
| tgaaaagaca acaaaaatat cttcatactt cctcatcccc tcattggaac aaaaccttaa | 2880 |
| actgggagaa ccttagtccc ctctctttcc tcttcctcct ccacttccca cttattgtca | 2940 |
| ccttgtaata ttcagagagc acttggatta tggatctgaa tagagaaatg cttacagata | 3000 |
| atcattagcc cacataccag taacttatac ttaaagatgg gatggagttg taaagtgctt | 3060 |
| ttataataca atataattgt taaaggcaag ggttgactct ttgttttatt ttgacatggc | 3120 |
| atgtcctgaa ataaatattg attcaatatg gcagatgggt catattcttt atttggaaga | 3180 |
| agttgtgact tctgacatgg gtgtgattgt cttcctacac tgttgcattt gattcttttt | 3240 |
| atgtattttt aagaaagtaa ccagttatac tgcttttaat attgattggt ctttttattt | 3300 |
| ggcttggagt tcttcaaagc attgaagtgt gttcatagtc caggtttttt ttttaataaa | 3360 |
| cacaattttg ctgccaaaaa tatataaata aaacacgaaa gaaaacaaaa aaaaaaaaa. | |

An example amino acid sequence of Rab27a is:

| (SEQ ID NO: 17) | |
|---|---|
| 1 | MSDGDYDYLI KFLALGDSGV GKTSVLYQYT DGKFNSKFIT TVGIDFREKR VVYRASGPDG |
| 61 | ATGRGORIHL QLWDTAGQER FRSLTTAFFR DAMGFLLLFD LTNEQSFLNV RNWISQLQMH |
| 121 | AYCENPDIVL CGNKSDLEDQ RVVKEEEAIA LAEKIGIPYF ETSAANGTNI SQAIEMLLDL |
| 181 | IMKRMERCVD KSWIPEGVVR SNGHASTDQL SEEKEKGACG C. |

Rab geranylgeranyltransferase (Rab GGTase)

Rab geranylgeranyltransferase (Rab GGTase; also known as geranylgeranyltransferase II) is a protein prenyltransferase which exclusively prenylates the GTPases of the Rab family.

Rab GGTase typically naturally catalyzes the transfer of two geranylgeranyl groups to cysteine residues at the C-terminus of Rab GTPases. Each geranylgeranyl group is conjugated to the Rab GTPase via a thioether linkage to a cysteine residue.

Rab GGTase has been shown to be capable of binding a range of derivatized phosphoisoprenoids and can catalyze their addition to Rab GTPase substrates (e.g. Rab6a).

For example, Nguyen et al. (Nguyen, U. T. et al, (2009) Nat. Chem. Biol. 5: 227-235) demonstrated the successful addition of a biotin-geranyl moiety to Rab GTPases.

Rab GGTase is a heterodimeric enzyme comprised of alpha and beta subunits.

In one embodiment, the Rab GGTase is human Rab GGTase. In a preferred embodiment, the Rab GGTase is rat Rab GGTase.

Example amino acid sequences of Rab GGTase alpha subunits are the sequences deposited under NCBI Accession Nos. NP_004572.3 (SEQ ID NO: 10) and NP_113842.1 (SEQ ID NO: 11).

Example amino acid sequences of Rab GGTase alpha subunits are:

(SEQ ID NO: 3)
MHGRLKVKTSEEQAEAKRLEREQKLKLYQSATQAVFQKRQAGELDESVLEL
TSQILGANPDFATLWNCRREVLQQLETQKSPEELAALVKAELGFLESCLRV
NPKSYGTWHHRCLLGRLPEPNWTRELELCARFLEVDERNFHCWDYRRFVAT
QAAVPPAEELAFTDSLITRNFSNYSSWHYRSCLLPQLHPQPDSGPQGRLPE
DVLLKELELVQNAFFTDPNDQSAWFYHRWLLGRADPQDALRCLHVSRDEAC
LTVSFSRPLLVGSRMEILLLMVDDSPLIVEWRTPDGRNRPSHVWLCDLPAA
SLNDQLPQHTFRVIWTAGDVQKECVLLKGRQEGQCRDSTTDEQLFRCELSV
EKSTVLQSELESCKELQELEPENKWCLLIILLMRALDPLLYEKETLQYFQT
LKAVDPMRATYLDDLRSKFLLENSVLKMEYAEVRVLHLAHKDLTVLCHLEQ
LLLVTHLDLSHNRLRTLPPALAALRCLEVLQASDNAIESLDGVTNLPRLQE
LLLCNNRLQQPAVLQPLASCPRLVLLNLQGNPLCQAVGILEQLAELLPSVS
SVLT

and:

(SEQ ID NO: 8)
MHGRLKVKISEEQAEAKRLEREQKLKLYQSATQAVFQKRQAGELDESVLE
LTSQILGANPDFATLWNCRREVLQHLETEKSPEESAALVEAELGFLESCL
RVNPKSYGTHHRCWLLSRLPEPNWARELELCARFLEADERNEHCWDYRRF
VAAQAAVAPAEELAFTDSLIIRNFSNYSSHYRSCLLPQLHPQPDSGPQGR
LPENVILKELELVONAFFIDPNDQSAWFYHRLLGRAEPHDVICCVHVSRE
EACLSVCFSRPLTVGSRMGTLLLMVDEAPLSVEWRTPDGRNRPSHVWLCD
LPAASLNDQLPQHTFRVIWTGSDSQKECVLLKDRPECWCRDSATDEQLFR
CELSVEKSTVLQSELESCKELQELEPENWCLLTIILLMRALDPLLYEKET
LQYFSTLKAVDPMRAAYLDDLRSKFLLENSVLKMEYADVRVLHLAHKDLT
VLCHLEQLLLVTHLDLSHNRLRALPPALAALRCLEVLQASDNALENVDGV
ANLPRLQELLLCNNRLQQSAAIQPLVSCPRLVLLNLQGNSLCQEEGIQER
LAEMLPSVSSILT

and:

(SEQ ID NO: 10)
MHGRLKVKISEEQAEAKRLEREQKLKLYQSATQAVFQKRQAGELDESVLEL
ISQILGANPDFATLWNCRREVLQQLETQKSPEELAALVKAELGFLESCLRV
NPKSYGTWHHRCWLLGRLPEPNWTRELELCARFLEVDERNFHCWDYRRFVA
TQAAVPPAEELAFTDSLITRNFSNYSSWHYRSCLLPQLHPQPDSGPQGRLP
EDVLLKELELVQNAFFIDPNDQSAWFYHRWLLGRADPQDALRCLHVSRDEA
CLTVSFSRPLLVGSRMEILLLMVDDSPLIVEWRTPDGRNRPSHVWLCDLPA
ASLNDQLPQHTFRVIWTAGDVQKECVLLKGRQEGWCRDSTTDEQLFRCELS
VEKSTVLQSELESCKELQELEPENKWCLLTIILLMRALDPLLYEKETLQYF
QTLKAVDPMRATYLDDLRSKFLLENSVLKMEYAEVRVLHLAHKDLTVLCHL
EQLLLVTHLDLSHNRLRTLPPALAALRCLEVLQASDNAIESLDGVTNLPRL
QELLLCNNRLQQPAVLQPLASCPRLVLLNLQGNPLCQAVGILEQLAELLPS
VSSVLT

and:

(SEQ ID NO: 11)
MHGRLKVKISEEQAEAKRLEREQKLKLYQSATQAVFQKRQAGELDESVLEL
ISQILGANPDFAILWNORREVLQHLETEKSPEESAALVKAELGFLESCLRV
NPKSYGTWHHROWLLSRLPEPNWARELELCARFLEADERNFHOWDYRRFVA
AQAAVAPAEELAFTDSLITRNESNYSSWHYRSOLLPQLHPQPDSOPQORLP
ENVLLKELELVQNAFFIDPNDQSAWFYHRWLLGRAEPHDVLOCVHVSREEA
CLSVCFSRPLIVOSRMGTLLLMVDEAPLSVEWRTPDORNRPSHVWLCDLPA
ASLNDQLPQHTFRVIWIGSDSQKECVLLKDRPECWORDSAIDEQLFRCELS
VEKSTVLQSELESOKELQELEPENKWCLLTIILLMRALDPLLYEKETLQYF
STLKAVDPMRAAYLDDLRSKFLLENSVLKMEYADVRVLHLAHKDLIVLOHL
EQLLLVIHLDLSHNRLRALPPALAALROLEVLQASDNALENVDOVANLPRL
QELLLCNNRLQQSAAIQPLVSCPRLVLLNLQGNSLOQEEGIQERLAEMLPS
VSSILT.

Example amino acid sequences of Rab GGTase beta subunits are the sequences deposited under NCBI Accession Nos. NP_004573.2 (SEQ ID NO: 4) and NP_619715.1 (SEQ ID NO: 12).

Example amino acid sequences of Rab GGTase beta subunits are:

(SEQ ID NO: 4)
MGTPQKDVIIKSDAPDTLLLEKHADYIASYGSKKDDYEYCMSEYLRMSGIY
WGLTVMDLMGQLHRMNREEILAFIKSCQHECGGISASIGHDPHLLYTLSAV
QILTLYDSINVIDVNKVVEYVKGLQKKEDGSFAGIWGEIDTRFSFCAVATL
ALLGKLDAINVEKAIEFVLSCMNFDGGFGCRPGSESHAGQIYCCTGFLAIT
SQLHQVNSDLLGWWLCERQLPSGGLNGRPEKLPDVCYSWWVLASLKIIGRL
HWIDREKLRNFILACQDEETGGFADRPGDMVDPFHTLFGIAGLSLLGEEQI
KPVNPVFCMPEEVLQRVNVQPELVS

and:

(SEQ ID NO: 9)
MGTQQKDVTIKSDAPDTLLLEKHADYIASYGSKKDDYEYSMSEYLRMSGVY
WGLTVMDLMGQLHRMNKEEILAFIKSCQHECGGVSASIGHDPHLLYTLSAV
QILTLYDSIHVINVDKVVAYVQSLQEDGSFAGDIGEIDTRFSFCAVATLAL
LGKLDAINVEKAIEFVLSCMNFDGGFGCRPGSESHAGQIYCCTGFLAITSQ
LHQVNSDLLGWWLCERQLPSGGLNGRPEKLPDVCYSWWVLASLKIIGRLHI
DREKLRSFILACQDEETGGFADRPGDMVDPFHTLFGIAGLSLLGEEQIKPV
SPVFCMPEEVLORVNVQPELVE

and:

(SEQ ID NO: 12)
MGTQQKDVTIKSDAPDTLLLEKHADYIASYGSKKDDYEYCMSEYLRMSGVY
WGLTVMDLMGQLHRMNKEEILVFIKSCQHECGGVSASIGHDPHLLYTLSAV
QILTLYDSIHVINVDKVVAYVQSLQKEDGSFAGDIWGEIDTRFSFCAVATL
ALLGKLDAINVEKAIEFVLSCMNFDGGFGCRPGSESHAGQIYCCTGFLAIT
SQLHQVNSDLLGWWLCERQLPSGGLNGRPEKLPDVCYSWWVLASLKIIGRL
HWIDREKLRSFILACQDEETGGFADRPGDMVDPFHTLFGIAGLSLLGEEQI
KPVSPVFCMPEEVLQRVNVQPELVS.

### Lipid Donor Substrate

To add a lipid moiety to a Rab GTPase, the Rab GGTase may use the lipid moiety in the form of a lipid (e.g. geranylgeranyl or biotin-geranyl) donor substrate as a substrate. These are typically pyrophosphate derivatives of the lipid moiety.

For example, geranylgeranylpyrophosphate (GGPP) or biotin-geranylpyrophosphate (BGPP) may be used as lipid donor substrates by Rab GGTase to transfer a geranylgeranyl or biotin-geranyl moiety, respectively, to the substrate Rab GTPase.

Geranylgeranylpyrophosphate has the structure:

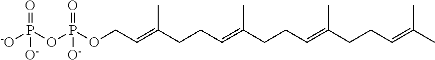

An example structure of biotin-geranylpyrophosphate is:

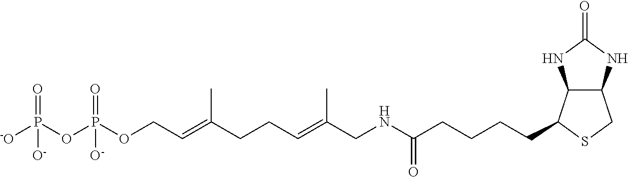

### Rab Escort Protein 1(REP1)

Rab escort proteins (REPs) perform the functions of presenting unprenylated Rab GTPases to Rab GGTases, and carrying prenylated Rab GTPases to their target membranes.

Rab GTPases do not comprise a consensus sequence at the prenylation site that may be recognized by Rab GGTases. However, substrate recognition is effected through REPs, which bind Rab GTPases through a conserved region and then present the Rab GGTase with its substrate for prenylation.

Once prenylated, the lipid anchors render the Rab GTPases insoluble. Accordingly, REPs are required to bind and solubilize the geranylgeranyl groups and aid delivery of the Rab GTPase to the target cell membrane.

REP1 may also be known as Rab protein geranylgeranyltransferase component A. Furthermore, the gene that encodes REP1 may be known as the CHM gene.

In one embodiment, the REP1 is human REP1.

An example amino acid sequence of REP1 is:

(SEQ ID NO: 5)
MADTLPSEFDVIVIGTGLPESIIAAACSRSGRRVLHVDSRSYYGGNWASFS
FSGLLSWLKEYQENSDIVSDSPVWQDQILENEEAIALSRKDTIQHVEVFCY
ASQDLHEDVEEAGALQKNHALVTSANSTEAADSAFLPTEDESLSTMSCEML
TEQTPSSDPENALEVNGAEVTGEKENHCDDKTCVPSTSAEDMSENVPIAED
TTEQPKKNRITYSQIIEGRRFNIDLVSKLLYSRGLLIDLLIKSNVSRYAEF
NITRILAFREGRVEQVPCSRADVFNSKQLTMVEKRMLMKFLTFCMEYEKY
PDEYGYEEITFYEYLKTQKLTPNLQYIVMHSIAMTSETASSTIDGLKATKN
FLHCLGRYGNTPFLFPLYGQGELPQCFCRMCAVFGGIYCLRHSVQCLVVDK
ESRKCKAIIDQFGQRIISEHFLVEDSYFPENMCSRVQYRQISRAVLITDRS
VLTDSDQQISILTVPAEEPGTFAVRVIELCSSTMTCMKGTYLVHLTCTSSK
TAREDLESVVQKLFVPYTEMEIENEQVEKPRILWALYFNMRDSSDISRSCY
NDLPSNVYVCSGPDCGLGNDNAVKQAETLFQEICPNEDFCPPPPNPEDIIL
DGDSLQPEASESSAIPEANSETFKESTNLGNLEESSE.

An example amino acid sequence of REP1 is:

(SEQ ID NO: 14)
MADTLPSEFDVIVIGTGLPESIIAAACSRSGRRVLHVDSRSYYGGNWASFS
FSGLLSWLKEYQENSDIVSDSPVWQDQILENEEAIALSRKDKTIQHVEVFC
YASQDLHEDVEEAGALQKNHALVTSANATEAADSAFLPTEDESLSTMSCEM
LTEQTPSSDPENALEVNGAEVTGEKENHCDDKTCVPSTSAEDMSENVPIAE
DTTEQPKKNRITYSQIIKEGRRFNIDLVSKLLYSRGLLIDLLIKSNVSRYA
EFKNITRILAFREGRVEQVPCSRADVFNSKQLTMVEKRMLMKFLTFCMEYE
KYPDEYKGYEEITFYEYLKTQKLTPNLQYIVMHSIAMTSETASSTIDGLKA
TKNFLHCLGRYGNTPFLFPLYGQGELPQCFCRMCAVFGGIYCLRHSVQCLV
VDKESRKCKAIIDQFGQRIISEHFLVEDSYFPENMCSRVQYRQISRAVLIT
DRSVLKTDSDQQISILTVPAEEPGTFAVRVIELCSSTMTCMKGTYLVHLTC
TSSKTAREDLESVVQKLFVPYTEMEIENEQVEKPRILWALYFNMRDSSDIS
RSCYNDLPSNVYVCSGPDCGLGNDNAVKQAETLFQEICPNEDFCPPPPNPE
DIILDGDSLQPEASESSAIPEANSETFKESTNLGNLEESSE.

An example nucleotide sequence encoding REP1 is:

(SEQ ID NO: 6)
ATGGCGGATACTCTCCCTTCGGAGTTTGATGTGATCGTAATAGGGACGGGTTTGCCTGAATC
CATCATTGCAGCTGCATGTTCAAGAAGTGGCCGGAGAGTTCTGCATGTTGATTCAAGAAGCT
ACTATGGAGGAAACTGGGCCAGTTTTAGCTTTTCAGGACTATTGTCCTGGCTAAAGGAATAC
CAGGAAAACAGTGACATTGTAAGTGACAGTCCAGTGTGGCAAGCCGATCCTTGAAAATGAAG
AGCCATTGCTCTTAGCAGGAAGGACAAAACATTCAACATGTGGAAGTATTTTGTTATGCCAG
TCAGGATTTGCATGAAGATGTCGAAGAAGCTGGTGCACTGCAGAAAAATCATGCTCTTGTGA
CATCTGCAAACTCCACAGAAGCTGCAGATTCTGCCTTCCTGCCTACGGAGGATGAGTCATTA
AGCACTATGAGCTGTGAAATGCTCACAGAACAAACTCCAAGCAGCGATCCAGAGAATGCGCT
AGAAGTAAATGGTGCTGAAGTGACAGGGGAAAAAGAAAACCATTGTGATGATAAAAGTTGTG
TGCCATCAACTTCAGCAGAAGACATGAGTGAAAATGTGCCTATAGCAGAAGATACCACAGAG
CAACCAAAGAAAAACAGAATTACTTACTCACAAATTATTAAAGAAGGCAGGAGATTTAATAT
TGATTTAGTATCAAAGCTGCTGTATTCTCGAGGATTACTAATTGATCTTCTAATCAAATCTA
ATGTTAGTCGATATGCAGAGTTTAAAAATATTACCAGGATTCTTGCATTTCGAGAAGGCGAG
TGGAACAGGTTCCGTGTTCCGGCGATGTCTTTAATAGCAAACAACTTACTATGGTAGAAAAG
CGAATGCTAATGAAATTTCTTACATTTTGTATGGAATATGAGAAATATCCTGATGAATAT
AAAGGATATGAAGAGATCACATTTTTGAATTTTAAAGACTCAAAAATTAACCCCCAACCTCC
AATATATTGTCATGCATTCAATTGCAATGACATCAGAGACAGCCAGCAGCACCATAGATGGT
CTCAAAGCTACCAAAAACTTTCTTCACTGTCTTGGGCGGTATGGCAACACTCCATTTTTGTT
TCCTTTATATGGCCAAGGAGAACTCCCCCAGTGTTTCTGCAGGATGTGTGCTGTGTTTGGTG
GAATTTATTGTCTTCGCCATTCAGTACAGTGCCTTGTAGTGGACAAAGAATCCAGAAAATGT
AAAGCAATTATAGATCAGTTTGGTCAGAGAATAATCTCTGAGCATTTCCTCGTGGAGGACAG
TTACTTTCCTGAGAACATGTGCTCACGTGTGCAATACAGGCAGATTCTTAGGGCAGTGCTGA
TTACAGAAGATCTGTCCTAAAAACAGATTCAGATCAACGATTTCCTTTTGACAGTGCCAGCA
GAGGAACCAGGAACTTTTGCTGTTCGGGTCATTGAGTTATGTTCTTCAACGATGACATGCAT
GAAAGGCACCTATTTGGTTCATTTGACTTGCACATCTTCTAAAACAGCAAGAGAAGATTTAG
AATCAGTTGTGCAGAAATTGTTTGTTCCATATACTGAAATGGAGATAGAAAATGAACAAGTA
GAAAAGCCAAGAATTCTGTGGGCTCTTTACTTCAATATGAGAGATTCGTCAGACATCAGCAG
GAGCTGTTATAATGATTTACCATCCAACGTTTATGTCTGCTCTGGCCCAGATTGTGGTTTAG
GAAATGATAATGCAGTCAAACAGGCTGAAACACTTTTCCAGGAAATCTGCCCCAATGAAGAT
TTCTGTCCCCCTCCCCAAATCCTGAAGACATTATCCTTGATGGAGACAGTTTACAGCCAGAG
GCTTCAGAATCCAGTGCCATACCAGAGGCTAACTCGGAGACTTTCAAGGAAAGCACAAACCT
TGGAAACCTAGAGGAGTCCTCTGAAAA

A further example nucleotide sequence encoding REP1 is:

(SEQ ID NO: 7)
GATATCGAATTCCTGCAGCCCGGCGGCACCATGGCGGATACTCTCCCTTCGGAGTTTGATGT
GATCGTAATAGGGACGGGTTTGCCTGAATCCATCATTGCAGCTGCATGTTCAAGAAGTGGCC
GGAGAGTTCTGCATGTTGATTCAAGAAGCTACTATGGAGGAAACTGGGCCAGTTTTAGCTTT
TCAGGACTATTGTCCTGGCTAAAGGAATACCAGGAAAACAGTGACATTGTAAGTGACAGTCC
AGTGTGGCAAGACCAGATCCTTGAAAATGAAGAAGCCATTGCTCTTAGCAGGAAGGACAAAA
CTATTCAACATGTGGAAGTATTTTGTTATGCCAGTCAGGATTTGCATGAAGATGTCGAAGAA
GCTGGTGCACTGCAGAAAAATCATGCTCTTGTGACATCTGCAAACTCCACAGAAGCTGCAGA
TTCTGCCTTCCTGCCTACGGAGGATGAGTCATTAAGCACTATGAGCTGTGAAATGCTCACAG
AACAAACTCCAAGCAGCGATCCAGAGAATGCGCTAGAAGTAAATGGTGCTGAAGTGACAGGG
GAAAAAGAAAACCATTGTGATGATAAAACTTGTGTGCCATCAACTTCAGCAGAAGACATGAG
TGAAAATGTGCCTATAGCAGAAGATACCACAGAGCAACCAAAGAAAAACGAATTACTTACTC
ACAAATATTAAGAAGGCAGGAGATTAATATTGATTTAGTATCAAAGCTGCTGTATTCTCGAG
GATTACTAATTGATCTTCTAATCAAATCTAATGTTAGTCGATATGCAGAGTTTAAAAATATT
ACCAGGATTCTTGCATTTCGAGAAGGACGAGTGGAACAGGTTCCGTGTTCCAGAGCAGATGT
CTTTAATAGCAAACAACTTACTATGGTAGAAAAGCGAATGCTAATGAAATTTCTTACATTTT
GTATGGAATATGAGAAATATCCTGATGAATATAAAGGATATGAAGAGATCACATTTTATGAA
TATTTAAAGACTCAAAAATTAACCCCCAACCTCCAATATATTGTCATGCATTCAATTGCAAT
GACATCAGAGACAGCCAGCAGCACCATAGATGGTCTCAAAGCTACCAAAAACTTTCTTCACT
GTCTTGGGCGGTATGGCAACACTCCATTTTTGTTTCCTTTATATGGCCAAGGAGAACTCCCC
CAGTGTTTCTGCAGGATGTGTGCTGTGTTTGGTGGAATTTATTGTCTTCGCCATTCAGTACA
GTGCCTTGTAGTGGACAAAGAATCCAGAAAATGTAAAGCAATTATAGATCAGTTTGGTCAGA
GAATAATCTCTGAGCATTTCCTCGTGGAGGACAGTTACTTTCCTGAGAACATGTGCTCACGT
GTGCAATACAGGCAGATCTCCAGGGCAGTGCTGATTACAGATAGATCTGTCCTAAAAACAGA
TTCAGATCAACAGATTTCCATTTTGACAGTGCCAGCAGAGGAACCAGGAACTTTTGCTGTTC
GGGTCATTGAGTTATGTTCTTCAACGATGACATGCTGAAAGGCACCTATTTGGTTCATTTGA
CTTGCACATCTTCTAAAACAGCAAGAGAAGATTTAGAATCAGTTGTGCAGAAATTGTTTGTT
CCATATACTGAAATGGAGATAGAAAATGAACAAGTAGAAAAGCCAAGAATTCTGTGGGCTCT
TTACTTCAATATGAGAGATTCGTCAGACATCAGCAGGAGCTGTTATAATGATTTACCATCCA
ACGTTTATGTCTGCTCTGGCCCAGATTGTGGTTTAGGAAATGATAATGCAGTCAAACAGGCT
GAAACACTTTTCCAGGAAATCTGCCCCAATGAAGATTTCTGTCCCCCTCCACCAAATCCTGA
AGACATTATCCTTGATGGAGACAGTTTACAGCCAGAGGCTTCAGAATCCAGTGCCATACCAG
AGGCTAACTCGGAGACTTTCAGGAAAGCACAAACCTTGGAAACCTAGAGGAGTCCTCTGAA
AA

A further example nucleotide sequence encoding REP1 is:

(SEQ ID NO: 15)
ATGGCGGATACTCTCCCTTCGGAGTTTGATGTGATCGTAATAGGGACGGGTTTGCCTGAATC
CATCATTGCAGCTGCATGTTCAAGAAGTGGCCGGAGAGTTCTGCATGTTGATTCAAGAAGCT
ACTATGGAGGAAACTGGGCCAGTTTTAGCTTTTCAGGACTATTGTCCTGGCTAAAGGAATAC
CAGGAAAACAGTGACATTGTAAGTGACAGTCCAGTGTGGCAAGACCAGATCCTTGAAAATGA
AGAAGCCATTGCTCTTAGCAGGAAGGACAAAACTATTCAACATGTGGAAGTATTTTGTTATG
CCAGTCAGGATTTGCATGAAGATGTCGAAGAAGCTGGTGCACTGCAGAAAAATCATGCTCTT
GTGACATCTGCAAACTCCACAGAAGCTGCAGATTCTGCCTTCCTGCCTACGGAGGATGAGTC
ATTAAGCACTATGAGCTGTGAAATGCTCACAGAACAAACTCCAAGCAGCGATCCAGAGAATG
CGCTAGAAGTAAATGGTGCTGAAGTGACAGGGGAAAAAGAAAACCATTGTGATGATAAAACT
TGTGTGCCATCAACTTCAGCAGAAGACATGAGTGAAAATGTGCCTATAGCAGAAGATACCAC
AGAGCAACCAAAGAAAAACAGAATTACTTACTCACAAATTATTAAAGAAGGCAGGAGATTTA
ATATTGATTTAGTATCAAAGCTGCTGTATTCTCGAGGATTACTAATTGATCTTCTAATCAAA
TCTAATGTTAGTCGATATGCAGAGTTTAAAAATATTACCAGGATTCTTGCATTTCGAGAAGG
ACGAGTGGAACAGGTTCCGTGTTCCAGACGCAGATGTCTTTAATAGCAAACAATTACTATGG
TAGAAAAGCGAATGCTAATGAAATTTCTTACATTTTGTATGGAATATGAGAAATATCCTGAT
GAATATAAAGGATATGAAGAGATCACATTTTATGAATATTTAAAGACTCAAAAATTAACCCC
CAACCTCCAATATATTGTCATGCATTCAATTGCAATGACATCAGAGACAGCCAGCAGCACCA
TAGATGGTCTCAAAGCTACCAAAAACTTTCTTCACTGTCTTGGGCGGTATGGCAACACTCCA
TTTTTGTTTCCTTTATATGGCCAAGGAGAACTCCCCCAGTGTTTCTGCAGGATGTGTGCTGT
GTTTGGTGGAATTTATTGTCTTCGCCATTCAGTACAGTGCCTTGTAGTGGACAAAGAATCCA
GAAAATGTAAAGCAATTATAGATCAGTTTGGTCAGAGAATAATCTCTGAGCATTTCCTCGTG
GAGGACAGTTACTTTCCTGAGAACATGTGCTCACGTGTGCAATACAGGCAGATCTCCAGGGC
AGTGCTGATTACAGATAGATCTGTCCTAAAAACAGATTCAGATCAACAGATTTCCATTTTGA
CAGTGCCAGCAGAGGAACCAGGAACTTTTGCTGTTCGGGTCATTGAGTTATGTTCTTCAACG
ATGACATGCATGAAAGGCACCTATTTGGTTCATTTGACTTGCACATCTTCTAAAACAGCAAG
AGAAGATTTAGAATCAGTTGTGCAGAAATTGTTTGTTCCATATACTGAAATGGAGATAGAAA
ATGAACAAGTAGAAAAGCCAAGAATTCTGTGGGCTCTTTACTTCAATATGAGAGATTCGTCA
GACATCAGCAGGAGCTGTTATAATGATTTACCATCCAACGTTTATGTCTGCTCTGGCCCAGA
TTGTGGTTTAGGAAATGATAATGCAGTCAAACAGGCTGAAACACTTTTCCAGGAAATCTGCC
CCAATGAAGATTTCTGTCCCCCTCCACCAAATCCTGAAGACATTATCCTTGATGGAGACAGT
TTACAGCCAGAGGCTTCAGAATCCAGTGCCATACCAGAGGCTAACTCGGAGACTTTCAAGGA
AAGCACAAACCTTGGAAACCTAGAGGAGTCCTCTGAATAA.

A further example nucleotide sequence encoding REP1 is:

(SEQ ID NO: 16)
GATATCGAATTCCTGCAGCCCGGCGGCACCATGGCGGATACTCTCCCTTCGGAGTTTGATGT
GATCGTAATAGGGACGGGTTTGCCTGAATCCATCATTGCAGCTGCATGTTCAAGAAGTGGCC
GGAGAGTTCTGCATGTTGATTCAAGAAGCTACTATGGAGGAAACTGGGCCAGTTTTAGCTTT
TCAGGACTATTGTCCTGGCTAAAGGAATACCAGGAAAACAGTGACATTGTAAGTGACAGTCC
AGTGTGGCAAGACCAGATCCTTGAAAATGAAGAAGCCATTGCTCTTAGCAGGAAGGACAAAA
CTATTCAACATGTGGAAGTATTTTGTTATGCCAGTCAGGATTTGCATGAAGATGTCGAAGAA
GCTGGTGCACTGCAGAAAAATCATGCTCTTGTGACATCTGCAAACTCCACAGAAGCTGCAGA
TTCTGCCTTCCTGCCTACGGAGGATGAGTCATTAAGCACTATGAGCTGTGAAATGCTCACAG
AACAAACTCCAAGCAGCGATCCAGAGAATGCGCTAGAAGTAAATGGTGCTGAAGTGACAGGG
GAAAAAGAAAACCATTGTGATGATAAAACTTGTGTGCCATCAACTTCAGCAGAAGACATGAG
TGAAAATGTGCCTATAGCAGAAGATACCACAGAGCAACCAAAGAAAAACAGAATTACTTACT
CACAAATTATTAAAGAAGGCAGGAGATTTAATATTGATTTAGTATCAAAGCTGCTGTATTCT
CGAGGATTACTAATTGATCTTCTAATCAAATCTAATGTTAGTCGATATGCAGAGTTTAAAAA
TATTACCAGGATTCTTGCATTTCGAGAAGGACGAGTGGAACAGGTTCCGTGTTCCAGAGCAG
ATGTCTTTAATAGCAAACAACTTACTATGGTAGAAAAGCGAATGCTAATGAAATTTCTTACA
TTTTGTATGGAATATGAGAAATATCCTGATGAATATAAAGGATATGAAGAGATCACATTTTA
TGAATATTTAAAGACTCAAAAATTAACCCCCAACCTCCAATATATTGTCATGCATTCAATTG
CAATGACATCAGAGACAGCCAGCAGCACCATAGATGGTCTCAAAGCTACCAAAAACTTTCTT
CACTGTCTTGGGCGGTATGGCAACACTCCATTTTTGTTTCCTTTATATGGCCAAGGAGAACT
CCCCCAGTGTTTCTGCAGGATGTGTGCTGTGTTTGGTGGAATTTATTGTCTTCGCCATTCAG
TACAGTGCCTTGTAGTGGACAAAGAATCCAGAAAATGTAAAGCAATTATAGATCAGTTTGGT
CAGAGAATAATCTCTGAGCATTTCCTCGTGGAGGACAGTTACTTTCCTGAGAACATGTGCTC
ACGTGTGCAATACAGGCAGATCTCCAGGGCAGTGCTGATTACAGATAGATCTGTCCTAAAAA
CAGATTCAGATCAACAGATTTCCATTTTGACAGTGCCAGCAGAGGAACCAGGAACTTTTGCT
GTTCGGGTCATTGAGTTATGTTCTTCAACGATGACATGCATGAAAGGCACCTATTTGGTTCA
TTTGACTTGCACATCTTCTAAAACAGCAAGAGAAGATTTAGAATCAGTTGTGCAGAAATTGT
TTGTTCCATATACTGAAATGGAGATAGAAAATGAACAAGTAGAAAAGCCAAGAATTCTGTGG
GCTCTTTACTTCAATATGAGAGATTCGTCAGACATCAGCAGGAGCTGTTATAATGATTTACC
ATCCAACGTTTATGTCTGCTCTGGCCCAGATTGTGGTTTAGGAAATGATAATGCAGTCAAAC
AGGCTGAAACACTTTTCCAGGAAATCTGCCCCAATGAAGATTTCTGTCCCCCTCCACCAAAT
CCTGAAGACATTATCCTTGATGGAGACAGTTTACAGCCAGAGGCTTCAGAATCCAGTGCCAT
ACCAGAGGCTAACTCGGAGACTTTCAAGGAAAGCACAAACCTTGGAAACCTAGAGGAGTCCT
CTGAATAA.

Example variants of REP1 are described further in WO 2012/114090 (incorporated herein by reference).

### Activity Determination

In one aspect, the invention provides a method for determining the activity of Rab escort protein 1 (REP1) comprising the steps: (a) providing a sample comprising REP1;(b) contacting the sample of step (a) with Rab6a, Rab geranylgeranyltransferase (Rab GGTase) and a lipid donor substrate; and (c) detecting the lipidated Rab6a product.

In another aspect, the invention provides the use of Rab6a for determining the activity of Rab escort protein 1 (REP1).

Assay sensitivity is an important factor to consider, because it enables detection of low levels of a target, which is particularly relevant when small quantities of reagents are present (e.g. as may be the case with gene therapy reagents). However, it is also important to maximize the dynamic range of an assay's signal, which may, for example, not correlate with reagents that provide low or high sensitivity.

The method and use of the invention are for testing the activity of REP1, rather than testing other agents that are involved in the prenylation of a Rab GTPase, for example, the activity of Rab GGTases or lipid donor substrates, or the activity of Rab GTPases as prenylation substrates. For example, the method of the invention may be for testing gene therapy vectors suitable for the delivery of REP1 to a target cell or for quality control analysis of vector stocks (e.g. medicament stocks).

In one embodiment, the sample comprising REP1 is from a cell genetically engineered to express the REP1. Preferably, a cell is transfected or transduced with a vector comprising a REP1-encoding nucleotide sequence to provide the cell genetically engineered to express the REP1. Preferably, the vector is a viral vector.

In one embodiment, the REP1 is expressed using a viral vector comprising a REP1-encoding nucleotide sequence.

The cell (which may be as a population of such cells) which is genetically engineered to express the REP1 may be any cell suitable for genetic engineering and expression of REP1, such as a cell from a cell line (e.g. HEK293). The cell may be, for example, a human or mouse cell. Preferably, the cell is a human cell. The cell may, for example, be a retinal cell, such as a retinal pigment epithelial or photoreceptor cell. In one embodiment, the cell is a HEK293 cell. In another embodiment, the cell is an ARPE-19 cell. In another embodiment, the cell is an HT1080 cell.

Preferably, the Rab6a and/or Rab GGTase are from a standard source such that they provide for minimal or no variation in repeated experiments. Preferably, the Rab6a and/or Rab GGTase are substantially pure (i.e. comprise substantially no protein contaminants that interfere with the method or use of the invention).

Accordingly, the method or use may comprise carrying out a plurality of experiments (e.g. comprising steps (a) to (c)) in which parameters relating to the sample comprising REP1 are varied, while other parameters (e.g. parameters relating to the Rab6a, Rab GGTase and lipid donor substrate) are kept constant. Such parameters may include, for example, the amino acid sequence of the relevant protein (e.g. REP1), the REP1-encoding nucleotide sequence comprised in a vector used to express the REP1 in a cell, the type of vector used to deliver a REP1-encoding nucleotide sequence to a cell (e.g the type of viral vector, such as the type of adeno-associated viral (AAV) vector), the concentration of REP1 and/or the multiplicity-of-infection (MOI) of a vector used to deliver a REP1-encoding nucleotide sequence to a cell.

The term “activity” is used herein to refer to the ability of REP1 to facilitate the lipidation of a Rab GTPase (e.g. Rab6a). Although the REP1 does not catalyze the lipidation itself, it is required for a Rab GGTase to catalyze the lipidation of its substrate Rab GTPase. Accordingly, the activity of the REP1 may be measured by determining the amount of Rab GTPase (i.e. Rab6a) which is lipidated under certain conditions.

The term “efficacy” is used herein, in relation to efficacy of a vector comprising a REP1-encoding nucleotide sequence, to refer to the ability of the vector to provide REP1 activity to a cell which is transfected or transduced by the vector.

The term “lipidated Rab6a product” as used herein refers to Rab6a to which a lipid moiety has been added. Preferably, the lipidated Rab6a product is a prenylated Rab6a, such as a geranylgeranylated Rab6a or a biotin-geranylated Rab6a.

Preferably, the step of detecting the lipidated Rab6a product provides quantification of the amount of lipidated Rab6a product.

The detection of lipidated Rab6a may be carried out by any suitable method, for example an enzyme-linked immunosorbent assay (ELISA), a Western blot, autoradiography (e.g. utilizing an isotopically-labelled, such as tritiated, lipid donor substrate), chromatographic (e.g. HPLC or FPLC) and/or mass spectrometry-based method (e.g. LC/MS).

In one embodiment, the lipidated Rab6a product is detected using a Western blot. In a preferred embodiment, the lipidated Rab6a product is detected using an ELISA.

By way of example, a prenylation reaction may be carried out according to the method of the invention using a biotin-geranylpyrophosphate lipid donor substrate. The product of the reaction may be subjected to Western blot analysis in which the lipidated Rab6a product (i.e. biotin-geranylated Rab6a) may be detected by direct incubation with, for example, streptavidin-horseradish peroxidase conjugate. Quantification of the lipidated Rab6a (i.e. biotin-geranylated Rab6a) may be achieved by densitometric analysis of the resulting Western blot, which may be carried out by any suitable means (e.g. using Image Studio Lite software (LI-COR)).

By way of further example, a prenylation reaction may be carried out according to the method of the invention using a biotin-geranylpyrophosphate lipid donor substrate. The product of the reaction may be subjected to an ELISA analysis in which the Rab6a may be immobilized on a plate directly or using an antibody that has been attached to the plate (i.e. a sandwich ELISA); and then the lipidated Rab6a product (i.e. biotin-geranylated Rab6a) may be detected by incubation with, for example, streptavidin-horseradish peroxidase conjugate. Quantification of the lipidated Rab6a (i.e. biotin-geranylated Rab6a) may be achieved by any suitable means (e.g. detection using a spectrophotometer, fluorometer or luminometer).

Further detection steps may be incorporated into the method of the invention, as required (e.g. for control purposes), such as the detection of the amount of REP1 present in the reaction or detection of the amount of β-actin (e.g. as a loading control).

In one embodiment, the method comprises a further step of comparing the amount of lipidated Rab6a product (e.g. prenylated, such as geranylgeranylated or biotin-geranylated, Rab6a) with an amount determined from a control experiment, such as an experiment using a known or standard sample of REP1.

In another embodiment, the method comprises a further step of comparing the amount of lipidated Rab6a product (e.g. prenylated, such as geranylgeranylated or biotin-geranylated, Rab6a) with a reference level.

Comparison with such control experiments or reference levels may provide a measure of the activity of the REP1 relative to a known or accepted standard (e.g. better or worse than a known or accepted standard).

The method of the invention may, for example, be used for quality control analysis of a gene therapy vector for the treatment of choroideremia, preferably an AAV vector particle comprising a REP1-encoding nucleotide sequence, wherein an output activity or efficacy of the vector determined by the method of the invention above a threshold activity or within a specified target range (e.g. by comparison to a control experiment or reference level) indicates the vector is suitable for gene therapy purposes.

The conditions of the prenylation reaction (e.g. that occurring in step (b) of the method of the invention) are not particularly limited, providing that they do not substantially interfere with the prenylation of Rab6a.

The sample comprising REP1 may be formulated in any suitable form, for example the sample may be prepared in a prenylation buffer comprising about 50 mM HEPES, 50 mM NaCl, 2 mM MgCl₂, 1 mM DTT and protease inhibitor cocktail (Roche) at about pH 7.5.

The sample comprising REP1 may, for example, comprise about 1-100, 1-75, 1-50, 1-40, 1-30, 1-20 or 1-10 μg of total protein. The sample comprising REP1 may, for example, comprise about 10-100, 10-75, 10-50, 10-40, 10-30 or 10-20 μg of total protein. Preferably, the sample comprising REP1 comprises about 10-30 μg of total protein, for example, about 10, 15, 20, 25 or 30 μg of total protein.

The Rab6a may, for example, be at a concentration of about 0.1-25, 0.1-20, 0.1-15, 0.1-10 or 0.1-5 μM, preferably about 0.1-5 μM, The Rab6a may, for example, be at a low concentration of about 0.1-1 μM. The Rab6a may, for example, be at a concentration of about 0.1, 0.2, 0.3, 0.4, 0.5, 0.6, 0.7, 0.8, 0.9, 1, 2, 3, 4, 5, 6, 7, 8, 9, 10, 12, 13, 14, 15, 16, 17, 18, 19, 20, 21, 22, 23, 24 or 25 μM, preferably about 4 μM. In certain embodiments, the Rab6a may, for example, be at a concentration of about 0.16 μM, 0.8 μM or 4 μM.

The Rab GGTase may, for example, be at a concentration of about 0.1-25, 0.1-20, 0.1-15, 0.1-10, 0.1-5 or 0.1-2.5 μM, preferably about 0.1-2.5 μM. The Rab GGTase may, for example, be at a concentration of about 0.1, 0.2, 0.3, 0.4, 0,5, 0.6, 0.7, 0,8, 0.9, 1, 2, 3, 4, 5, 6, 7, 8, 9, 10, 11, 12, 13, 14, 15, 16, 17, 18, 19, 20, 21, 22, 23, 24 or 25 μM, preferably about 2 μM. In certain embodiments, the Rab GGTase may, for example, be at a concentration of about 0.5 μM, 1 μM or 2 μM.

The lipid donor substrate (e.g. biotin-geranylpyrophosphate (BGPP)) may, for example, be at a concentration of about 1-25, 1-20, 1-15, 1-10 or 1-5 μM, preferably about 1-5 μM. The lipid donor substrate (e.g. ⁻biotin-geranylpyrophosphate (BGPP)) may, for example, be at a concentration of about 1, 2, 3, 4, 5, 6, 7. 8, 9, 10, 11, 12, 13, 14, 15, 16, 17, 18, 19, 20, 21, 22, 23, 24 or 25 μM, preferably about 4 μM.

The prenylation reaction may be carried out in any suitable buffer, for example the reaction may be carried out in a prenylation buffer comprising about 50 mM HEPES, 50 mM NaCl, 2 mM MgCl2, 1 mM DTT and protease inhibitor cocktail (Roche) at about pH 7.5.

Prenylation reactions may be carried out for any suitable length of time at any suitable temperature (e.g. about 37° C.). For example, prenylation reactions may be carried out for about 1-10, 1-7.5, 1-5, 1-2.5 or 1-2 h, preferably about 1-2 h. Prenylation reactions may, for example, be carried out for about 1, 2, 3, 4, 5, 6, 7, 8, 9 or 10 h, preferably about 2 h.

### Choroideremia

Choroideremia is a rare X-linked progressive degeneration of the choroid, retinal pigment epithelium and photoreceptors of the eye. The typical natural history in afflicted males is onset of nightblindness during teenage years, and then progressive loss of peripheral vision during the 20's and 30's leading to complete blindness in the 40's. Female carriers have mild symptoms, most notably nightblindness, but may occasionally have a more severe phenotype.

Choroideremia is caused by mutations in the CHM gene, which is located on the X chromosome 21q region. Rah escort protein 2 (REP2), which is 75% homologous to REP1, compensates for any REP1 deficiency in most cells of the body. However, for reasons that are not yet clear, REP2 is unable to compensate for REP1 deficiency in the eye. This leads to insufficient Rab escort protein activity to maintain normal prenylation of target Rab GTPases and gives rise to cellular dysfunction and ultimately cell death, primarily affecting the outer retina and choroid.

Choroideremia may be successfully treated by providing functional copies of the REP1 transgene to the affected cells of the eye (MacLaren, R. E. et al. (2014) Lancet 383: 1 129-37).

### Vectors

A vector is a tool that allows or facilitates the transfer of an entity from one environment to another. In accordance with the invention, and by way of example, some vectors used in recombinant nucleic acid techniques allow entities, such as a segment of nucleic acid (e.g. a heterologous DNA segment, such as a heterologous cDNA segment), to be transferred into a target cell. The vector may serve the purpose of maintaining the heterologous nucleic acid (e.g. DNA or RNA) within the cell, facilitating the replication of the vector comprising a segment of nucleic acid or facilitating the expression of the protein encoded by a segment of nucleic acid.

Vectors may be non-viral or viral. Examples of vectors used in recombinant nucleic acid techniques include, but are not limited to, plasmids, chromosomes, artificial chromosomes and viruses. The vector may also be, for example, a naked nucleic acid (e.g. DNA or RNA). In its simplest form, the vector may itself be a nucleotide of interest.

The vectors used in the invention may be, for example, plasmid or viral vectors and may include a promoter for the expression of a polynucleotide and optionally a regulator of the promoter.

### Viral Vectors

In a preferred embodiment, the vector of the invention is a viral vector. Preferably, the viral vector is in the form of a viral vector particle.

The viral vector may be, for example, an adeno-associated viral (AAV), retroviral, lentiviral or adenoviral vector. Preferably, the viral vector is an AAV vector.

The term “gene therapy vector” is used herein to refer to a vector which is suitable for use in gene therapy and includes, for example, viral (e.g. AAV) vectors and vector particles.

In some embodiments, viral vectors and vector particles of the disclosure may be used in gene therapy. It is important that the viral vectors and vector particles of the disclosure maintain biocompatibility and stability following storage and passage through injection devices for AAV gene therapy. In some embodiments, the viral vectors and vector particles of the disclosure may be diluted in TMN 200 buffer to maintain biocompatibility and stability. TMN 200 buffer comprises 20 mM Tris (pH adjusted to 8.0), 1 mM MgCl₂ and 200 mM NaCl.

The determination of the physical viral genome titer is part of the characterization of the vector and is a step to ensure potency and safety of viral vectors and viral particles during gene therapy. In some embodiments, a method to determine the AAV titer comprises quantitative PCR (qPCR). There are different variables that can influence the results, such as the conformation of the DNA used as standard or the enzymatic digestion during the sample preparation. For example, the viral vector or particle preparation whose titer is to be measured can be compared against a standard dilution curve generated using a plasmid. In some embodiments, the plasmid DNA used in the standard curve is in the supercoiled conformation. In some embodiments, the plasmid DNA used in the standard curve is in the linear conformation. Linearized plasmid can be prepared, for example by digestion with HindIII restriction enzyme, visualized by agarose gel electrophoresis and purified using the QIAquick Gel Extraction Kit (Qiagen) following manufacturer's instructions. Other restriction enzymes that cut within the plasmid used to generate the standard curve may also be appropriate. In some embodiments, the use of supercoiled plasmid as the standard significantly increased the titre of the AAV vector compared to the use of linearized plasmid.

To extract the DNA from purified AAV vectors for quantification of AAV genome titer, two enzymatic methods can be used. In some embodiments, the AAV vector may be singly digested with DNase I. In some embodiments, the AAV vector may be and double digested with DNase I and an additional proteinase K treatment. QPCR can then performed with the CFX. Connect Real-Time PCR Detection System (BioRad) using primers and Taqman probe specific to the transgene sequence.

### Variants, Derivatives, Analogues, Homologues and Fragments

In addition to the specific proteins and nucleotides mentioned herein, the invention also encompasses the use of variants, derivatives, analogues, homologues and fragments thereof.

In the context of the invention, a variant of any given sequence is a sequence in which the specific sequence of residues (whether amino acid or nucleic acid residues) has been modified in such a manner that the polypeptide or polynucleotide in question substantially retains its function. A variant sequence can be obtained by addition, deletion, substitution, modification, replacement and/or variation of at least one residue present in the naturally-occurring protein.

The term “derivative” as used herein, in relation to proteins or polypeptides of the invention includes any substitution of, variation of, modification of, replacement of, deletion of and/or addition of one (or more) amino acid residues from or to the sequence providing that the resultant protein or polypeptide substantially retains at least one of its endogenous functions.

The term “analogue” as used herein, in relation to polypeptides or polynucleotides includes any mimetic, that is, a chemical compound that possesses at least one of the endogenous functions of the polypeptides or polynucleotides which it mimics.

Typically, amino acid substitutions may be made, for example from 1, 2 or 3 to 10 or 20 substitutions provided that the modified sequence substantially retains the required activity or ability. Amino acid substitutions may include the use of non-naturally occurring analogues.

Proteins used in the invention may also have deletions, insertions or substitutions of amino acid residues which produce a silent change and result in a functionally equivalent protein. Deliberate amino acid substitutions may be made on the basis of similarity in polarity, charge, solubility, hydrophobicity, hydrophilicity and/or the amphipathic nature of the residues as long as the endogenous function is retained. For example, negatively charged amino acids include aspartic acid and glutamic acid; positively charged amino acids include lysine and arginine; and amino acids with uncharged polar head groups having similar hydrophilicity values include asparagine, glutamine, serine, threonine and tyrosine.

Conservative substitutions may be made, for example according to the table below. Amino acids in the same block in the second column and preferably in the same line in the third column may be substituted for each other:

| | | |
|---|---|---|
| ALIPHATIC | Non-polar | GAP |
| | | ILV |
| | Polar - uncharged | CSTM |
| | | NQ |
| | Polar - charged | DE |
| | | KRH |
| AROMATIC | | FWY |

The term “homologue” as used herein means an entity having a certain homology with the wild type amino acid sequence and the wild type nucleotide sequence. The term “homology” can be equated with “identity”.

A homologous sequence may include an amino acid sequence which may be at least 50%, 55%, 60%, 65%, 70%, 75%, 80%, 85% or 90% identical, preferably at least 95% or 97% or 99% identical to the subject sequence. Typically, the homologues will comprise the same active sites etc. as the subject amino acid sequence. Although homology can also be considered in terms of similarity (i.e. amino acid residues having similar chemical properties/functions), in the context of the invention it is preferred to express homology in terms of sequence identity.

A homologous sequence may include a nucleotide sequence which may be at least 50%, 55%, 60%, 65%, 70%, 75%, 80%, 85% or 90% identical, preferably at least 95% or 97% or 99% identical to the subject sequence. Although homology can also be considered in terms of similarity, in the context of the invention it is preferred to express homology in terms of sequence identity.

Preferably, reference to a sequence which has a percent identity to any one of the SEQ ID NOs detailed herein refers to a sequence which has the stated percent identity over the entire length of the SEQ ID NO referred to.

Homology comparisons can be conducted by eye or, more usually, with the aid of readily available sequence comparison programs. These commercially available computer programs can calculate percent homology or identity between two or more sequences.

Percent homology may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion in the nucleotide sequence may cause the following codons to be put out of alignment, thus potentially resulting in a large reduction in percent homology when a global alignment is performed. Consequently, most sequence comparison methods are designed to produce optimal alignments that take into consideration possible insertions and deletions without penalizing unduly the overall homology score. This is achieved by inserting “gaps” in the sequence alignment to try to maximize local homology,

However, these more complex methods assign “gap penalties” to each gap that occurs in the alignment so that, for the same number of identical amino acids, a sequence alignment with as few gaps as possible, reflecting higher relatedness between the two compared sequences, will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimized alignments with fewer gaps. Most alignment programs allow the gap penalties to be modified. However, it is preferred to use the default values when using such software for sequence comparisons. For example when using the GCG Wisconsin Bestfit package the default gap penalty for amino acid sequences is −12 for a gap and −4 for each extension.

Calculation of maximum percent homology therefore firstly requires the production of an optimal alignment, taking into consideration gap penalties. A suitable computer program for carrying out such an alignment is the GCG Wisconsin Bestfit package (University of Wisconsin, U.S.A.; Devereux et al. (1984) Nucleic Acids Res. 12: 387). Examples of other software that can perform sequence comparisons include, but are not limited to, the BLAST package (see Ausubel et al. (1999) ibid—Ch. 18), FASTA (Atschul et al. (1990) J. Mol. Biol. 403-410) and the GENEWORKS suite of comparison tools. Both BLAST and FASTA are available for offline and online searching (see Ausubel et al. (1999) ibid., pages 7-58 to 7-60). However, for some applications, it is preferred to use the GCG Bestfit program. Another tool, called BLAST 2 Sequences is also available for comparing protein and nucleotide sequences (see FEMS Microbiol. Lett. (1999) 174: 247-50; and FEMS Microbiol. Lett. (1999) 177: 187-8).

Although the final percent homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see the user manual for further details). For some applications, it is preferred to use the public default values for the GCG package, or in the case of other software, the default matrix, such as BLOSUM62.

Once the software has produced an optimal alignment, it is possible to calculate percent homology, preferably percent sequence identity. The software typically does this as part of the sequence comparison and generates a numerical result.

“Fragments” of full length polypeptides or polynucleotides of the invention are also variants and the term typically refers to a selected region of the polypeptide or polynucleotide that is of interest either functionally or, for example, in an assay. “Fragment” thus refers to an amino acid or nucleic acid sequence that is a portion of a full-length polypeptide or polynucleotide.

Such variants may be prepared using standard recombinant DNA techniques such as site-directed mutagenesis. Where insertions are to be made, synthetic DNA encoding the insertion together with 5′ and 3′ flanking regions corresponding to the naturally-occurring sequence either side of the insertion site may be made. The flanking regions will contain convenient restriction sites corresponding to sites in the naturally-occurring sequence so that the sequence may be cut with the appropriate enzyme(s) and the synthetic DNA ligated into the cut. The DNA is then expressed in accordance with the invention to make the encoded protein. These methods are only illustrative of the numerous standard techniques known in the art for manipulation of DNA sequences and other known techniques may also be used.

### Codon Optimization

The polynucleotides used in the present invention may be codon-optimized. Codon optimization has previously been described in WO 1999/41397 and WO 2001/79518. Different cells differ in their usage of particular codons. This codon bias corresponds to a bias in the relative abundance of particular tRNAs in the cell type. By altering the codons in the sequence so that they are tailored to match with the relative abundance of corresponding tRNAs, it is possible to increase expression. By the same token, it is possible to decrease expression by deliberately choosing codons for which the corresponding tRNAs are known to be rare in the particular cell type. Thus, an additional degree of translational control is available.

### EXAMPLES

### Materials and Methods (Examples 1-2)

### Cell Transduction and Harvesting

Cultured HEK293 cells were treated with rAAV2/2-REP1 at a range of multiplicities of infection (MOI, genome particles/cell). rAAV2/2-GFP was used in parallel as a control vector, and fluorescence was monitored for onset of transgene expression.

Experiments on untransduced, and +AAV-GFP transduced and +AAV-REP1 transduced cells were conducted in parallel.

Cell lysates were prepared at day 5 post-transduction using the following protocol: cells were washed with PBS and incubated for 5 min with prenylation buffer, pH 7.5 (50 mM HEPES, 50 mM NaCl, 2 mM MgCl₂, 1 mM DTT and protease inhibitor cocktail (Roche)) on ice; cells were then scraped using a cell scraper into a 1.5 mL tube and incubated on ice for 15 mM; subsequently, cells were disrupted by pushing them 20 times through a 26-G syringe needle attached to a 1 mL syringe.

Lysed cells were centrifuged for 5 mM at 1500×g at 4° C. The supernatant was then transferred to cellulose propionate tubes and centrifuged for 1 h at 100000×g at 4° C. The supernatant from the second centrifugation step was used for the in vitro prenylation reactions (described below).

### Total Protein Quantification

Total cell protein concentration was quantified using the Bradford method according to the manufacturer's instructions (Quick Start™ Bradford 1× Dye Reagent, BioRad, #500-0205). Sample values were extrapolated from a standard curve.

### In Vitro Prenylation Reaction

Prenylation reactions were set up using frozen cell lysate (10-30 μg), 2 μM Rab GGTase, 40 μM Rab protein (Rab27a or Rab6a) and 5 μM biotin-geranylpyrophosphate (BGPP) as the lipid donor, in prenylation buffer. All reactions were supplemented with fresh GDP (guanosine diphosphate, 20 μM) and DTT (1 mM).

For positive control samples, fish REP1 (see individual experiments for the amount) was added to the prenylation reaction containing lysate from untransduced cells.

Reactions were incubated for 2 h at 37° C. and then stopped by addition of sample buffer (Laemmli buffer, 2× concentrate, Sigma #S3401). This buffer contains 4% SDS, 20% glycerol, 10% 2-mercaptoethanol 0.004% bromphenol blue and 0.125 M Tris HCl, pH approx. 6.8.

Western blots (WB) were performed to detect human REP-1, β-actin (as a loading control) and biotinylated. Rab protein (Rab27a or Rab6a).

For detection of human REP1, a mouse monoclonal antibody from Millipore was used (clone 2F1, #MABN52). For detection of β-actin, a mouse monoclonal antibody from Thermo Fisher Scientific was used (clone AC-15, #AM4302). Both detections were followed by a secondary antibody-labelling step (donkey anti-mouse HRP, Abeam, #ab98799).

The incorporation of biotinylated lipid donor into the appropriate Rab substrate was detected by direct incubation with streptavidin-HRP (Thermo Fisher Scientific, #43-4323).

All membranes were detected using ECL substrate and Odyssey FC detection system (Ll-COR). The intensities of the bands were quantitatively analyzed using Image Studio Lite software (LI-COR).

### Example 1

### Rab27a as a Prenylation Substrate

To test the sensitivity of a prenylation assay using Rab27a as a substrate, experiments were carried out in parallel using the following cells: (a) untransduced cells; (b) cells transduced with AAV-GFP at a MOI of 10000; and (c) cells transduced with AAV-REP1 at a MOI of 10,000.

Prenylation reactions were set up using 10 μg of lysate in a total volume of 12.5 μL. Positive controls were spiked with 2 μM of fish REP1.

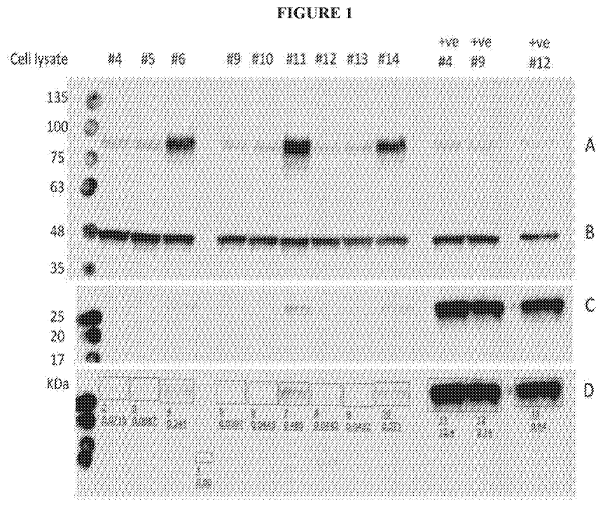

The results indicate that Rab27a is a substrate for the prenylation assay to assess REP1 function following transduction of cells with AAV-REP1 (FIG. 1). However, the signal from the WB semiquantification is not very strong.

This study was repeated with an increased amount of total cell protein to increase the WB band intensity.

Prenylation reactions were set up using 30 μg of lysate in a total volume of 22 μL. Positive controls were spiked with 1 μM of fish REP1.

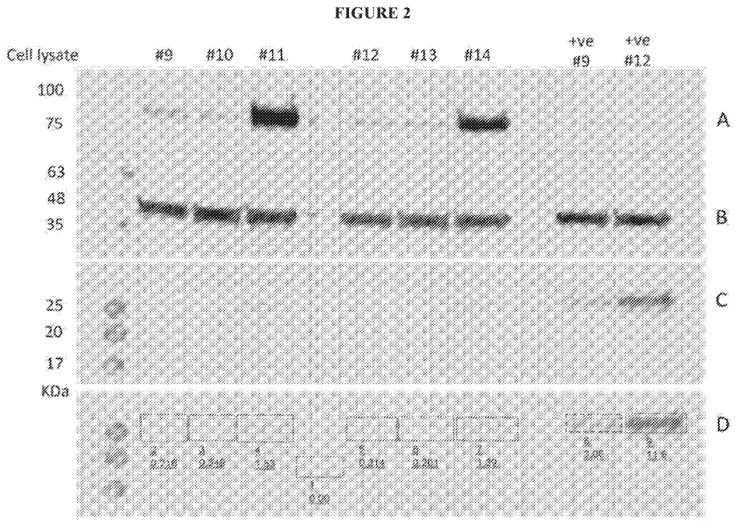

The results confirm that Rab27a works as a substrate for the prenylation assay to assess REP1 function following transduction of cells with AAV-REP 1 (FIG. 2). Furthermore, the strength of the WB signal has increased compared to the data obtained using 10 μg of lysate. However, the signal is still not very strong. Ideally, a larger increase of prenylated Rab protein when cells are transduced with AAV-REP1 would be observed.

### Example 2

### Rab6a as a Prenylation Substrate

To test the sensitivity of a prenylation assay using Rah6a as a substrate, experiments were carried out in parallel using the following cells (same cell lysates used in Example 1): (a) untransduced cells; (b) cells transduced with AAV-GFP at a MOI of 10,000; and (c) cells transduced with AAV-REP1 at a MOI of 10,000. Prenylation reactions were set up using 20 μg of lysate in a total volume of 20 μL Positive controls were spiked with 1 μM of fish REP1.

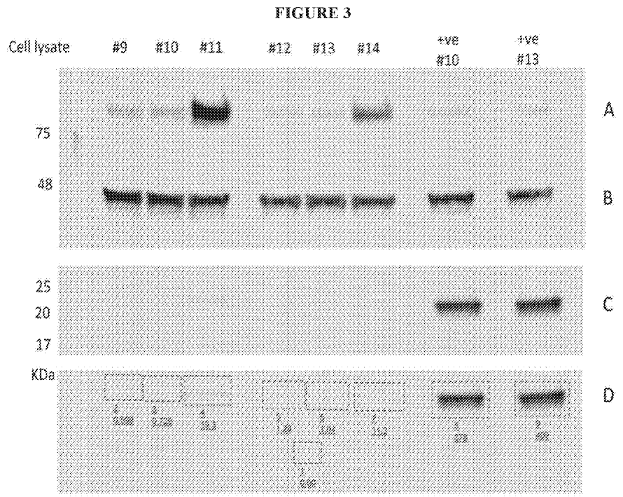

The results indicate that Rab6a is an effective substrate for the prenylation assay to assess REP1 function following transduction of cells with AAV-REP1 (FIG. 3).

The strength of the WB signal has increased approximately 10-fold for AAV-REP1 transduced cells, compared to the data shown in FIG. 2, even though less total protein was used. Furthermore, the band intensity for the positive controls is approximately 100-fold greater compared to the data shown in FIG. 2, confirming the increased sensitivity of the Rab6a-based assays.

The data also demonstrate the increased sensitivity enables the detection of differences at endogenous levels too, which makes the assay more accurate.

Following the successful demonstration of increased assay sensitivity provided by the use of Rab6a as the prenylation substrate, the assay was repeated using different MOIs of AAV-REP1 study whether Rab6a, prenylation correlates with the amount of AAV-REP1.

Experiments were carried out in parallel using the following cells: (a) untransduced cells; (b) cells transduced with AAV-REP1 at a MOI of 250; (c) cells transduced with AAV-REP1 at a MOI of 1000; (d) cells transduced with AAV-REP1 at a MOI of 5000; (e) cells transduced with AAV-REP1 at a MOI of 10,000; and (f) cells transduced with AAV-REP1 at a MOI of 20,000.

Prenylation reactions were set up using 20 μg of lysate in a total volume of 15 μL. The positive control was spiked with 0.5 μM of fish REP1.

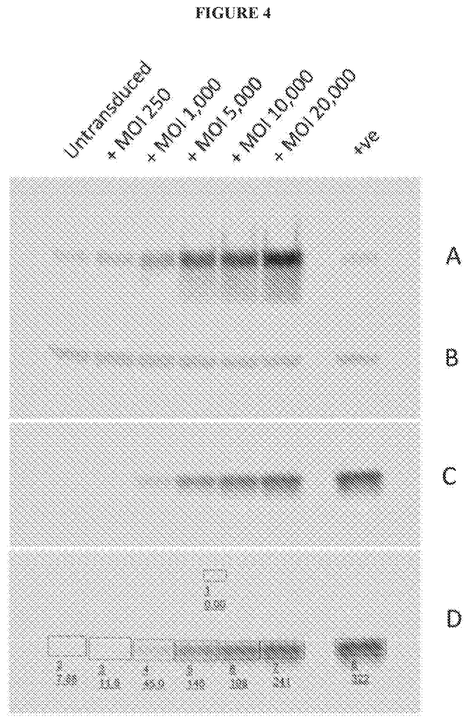

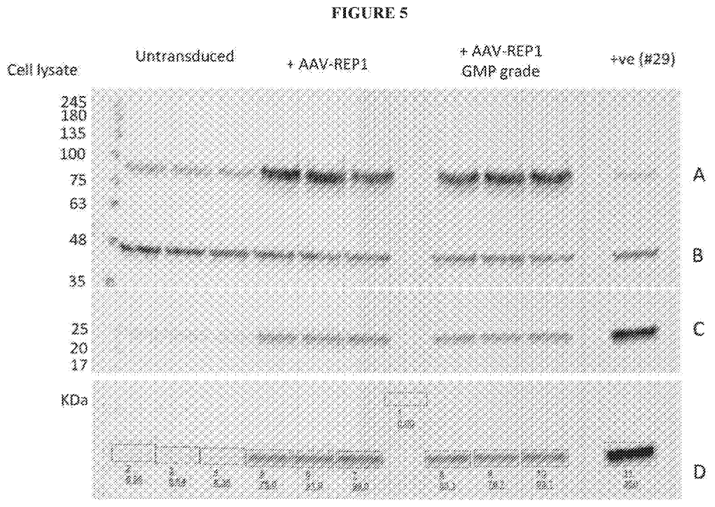

The results confirm that Rab6a is an effective substrate for the prenylation assay to assess REP1 function following transduction of cells with AAV-REP1 (FIG. 4) and furthermore demonstrate that the incorporation of biotinylated lipid donor in Rab6a correlates with the amount of AAV-REP1 used for cell transduction.

Following the validation of Rab6a as an effective assay substrate, we then tested the AAV-REP1 vector currently in use in our Phase 1 clinical trial (MacLaren, R. E. et al. (2014) Lancet 383: 1 129-37).

Experiments were carried out in parallel using the following cells: (a) untransduced cells (#29, #30 and #31); (b) cells transduced with AAV-REP1 at a MOI of 10,000 (#32, #33 and #34); and (c) cells transduced with GMP grade AAV-REP1 at a MOI of 10,000 (#35, #36 and #37).

Prenylation reactions were set up using 20 μg of lysate in a total volume of 15 μL. The positive control was spiked with 0.5 μM of fish REP1.

The results are in keeping with the previous experiments and confirm that the incorporation of biotinylated lipid donor in Rab6a correlates with the amount of AAV-REP1 used for cell transduction (FIG. 5).

### Example 3

### Rab6a as a Substrate in the Prenylation Reaction Using ARPE-19 Cells Cell Transduction and Harvesting

Cultured ARPE-19 cells were treated with rAAV2/2-REP1 at an MOI of 10,000 genome particles/cell. Cell lysates were prepared at day 13 post-transduction: cells were washed with PBS and incubated with prenylation buffer, pH 7.5 (50 mM HEPES, 50 mM NaCl, 2 mM MgCl₂, 1 mM DTT and protease inhibitor cocktail (Roche)) on ice. Cells were scraped using a cell scraper into a 1.5 mL tube, and incubated on ice for 15 min. Cells were disrupted by pushing them 20 times through a 26-G syringe needle attached to a 1 mL syringe. Cells were spun for 5 min at 1,500×g, 4° C. The supernatant was then transferred to cellulose propionate tubes and centrifuged at 100,000×g for 1 h at 4° C. The supernatant was used for the in vitro prenylation reaction.

### Total Protein Quantification

Total cell protein was quantified using the Bradford method according to the manufacturer's instructions (Quick Start™ Bradford 1× Dye Reagent, BioRad, #500-0205). Sample values were extrapolated from a standard curve.

### In Vitro Prenylation Reaction

The prenylation reactions were set up using frozen cell lysate (15 μg), 2 μM Rab GGTase, 4 μM of Rab protein (Rab6a) and 5 μM of biotin-geranylpyrophosphate as lipid donor, in prenylation buffer. All reactions were supplemented with fresh GDP (20 μM) and DTT (1 mM). In positive control samples, fish REP1 (see experiments for amount) was added to the prenylation reaction containing untransduced cell lysate.

The reactions were incubated for 2 h at 37° C. and then stopped by addition of SDS-PAGE sample buffer.

Western blotting (WB) was performed to detect human REP-1, β-actin (loading control) and biotinylated Rab protein (Rab27a or Rab6a). For detection of human REP1, a mouse monoclonal antibody from Millipore was used (clone 2F1, #MABN52). For detection of β-actin, a mouse monoclonal antibody from Thermo Fisher Scientific was used (clone AC-15, #AM4302). Both detections were followed by a secondary antibody-labelling step (donkey anti-mouse HRP, Abcam, #ab98799). The incorporation of biotinylated lipid donor into the appropriate Rab substrate was detected by direct incubation with streptavidin-HRP (Thermo Fisher Scientific, #43-4323). All membranes were detected using ECL substrate and Odyssey FC detection system (LI-COR). The intensities of the bands were quantitatively analyzed using Image Studi Lite software (LI-COR).

### Results and Discussion

To test the prenylation assay using Rab6a as a substrate in ARPE-19 cells (human retinal pigment epithelium cells), experiments were carried out in parallel using the following cells: (a) Untransduced cells (#86 and #87); and (b) Cells+AAV-REP1 MOI 10,000 (#90 and #91)—R&D grade vector. Prenylation reactions were set up using 15 μg of lysate in a total volume of 45 μL. Positive control was spiked with 0.1 μM of fish REP1.

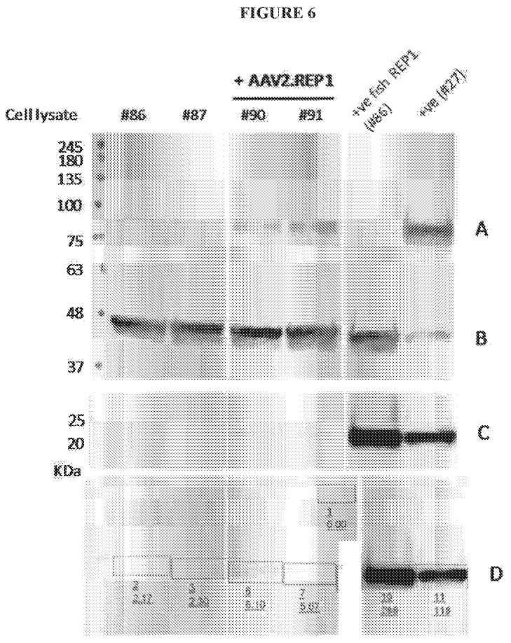

The results indicate that Rab6a works as a substrate for the prenylation assay to assess REP1 function following transduction of ARPE-19 cells with AAV-REP1 (FIG. 6).

### Example 4

### Rab6a as a Substrate in the Prenylation Reaction Using HT1080 Cells

### Cell Transduction and Harvesting

Cultured HT1080 cells were treated with rAAV2/2-REP1 at an MOI of 10,000 genome particles/cell. Cell lysates were prepared at day 5 post-transduction: cells were washed with PBS and incubated with prenylation buffer, pH 7.5 (50 mM HEPES, 50 mM NaCl, 2 mM MgCl₂, 1 mM DTT and protease inhibitor cocktail (Roche)) on ice. Cells were scraped using a cell scraper into a 1.5 mL tube, and incubated on ice for 15 min. Cells were disrupted by pushing them 20 times through a 26-G syringe needle attached to a 1 mL syringe. Cells were spun for 5 min at 1,500×g, 4° C. The supernatant was then transferred to cellulose propionate tubes and centrifuged at 100,000×g for 1 h at 4° C. The supernatant was used for the in vitro prenylation reaction.

### Total Protein Quantification

Total cell protein was quantified using the Bradford method according to the manufacturer's instructions (Quick Start™ Bradford 1× Dye Reagent, BioRad, #500-0205). Sample values were extrapolated from a standard curve.

### In Vitro Prenylation Reaction

The prenylation reactions were set up using frozen cell lysate (20 μg), 2 μM Rab GGTase, 4 μM of Rab protein (Rab6a) and 5 μM of biotin-geranylpyrophosphate as lipid donor, in prenylation buffer. All reactions were supplemented with fresh GDP (20 μM) and DTT (1 mM). In positive control samples, fish REP1 (see experiments for amount) was added to the prenylation reaction containing untransduced cell lysate.

The reactions were incubated for 2 h at 37° C. and then stopped by addition of SDS-PAGE sample buffer.

Western blotting (WB) was performed to detect human REP-1, β-actin (loading control) and biotinylated Rab protein (Rab27a or Rab6a). For detection of human REP1, a mouse monoclonal antibody from Millipore was used (clone 2F1, #MABN52). For detection of β-actin, a mouse monoclonal antibody from Thermo Fisher Scientific was used (clone AC-15, #AM4302). Both detections were followed by a secondary antibody-labelling step (donkey anti-mouse HRP, Abcam, #ab98799). The incorporation of biotinylated lipid donor into the appropriate Rab substrate was detected by direct incubation with streptavidin-HRP (Thermo Fisher Scientific, #43-4323). All membranes were detected using ECL substrate and Odyssey FC detection system (LI-COR). The intensities of the bands were quantitatively analyzed using Image Studi Lite software (LI-COR).

To test the prenylation assay using Rab6a as a substrate in HT1080 cells experiments were carried out in parallel using the following cells: (a) Untransduced cells (#56 and #57) (b) Cells+AAV-REP1 MOI 10,000 (#60 and #61)—R&D grade vector; and (c) Cells+AAV-REP1 MOI 10,000 (#64 and #65)—clinical grade vector.

Prenylation reactions were set up using 20 μg of lysate in a total volume of 20 μL. Positive control was spiked with 0.1 μM of fish REP 1.

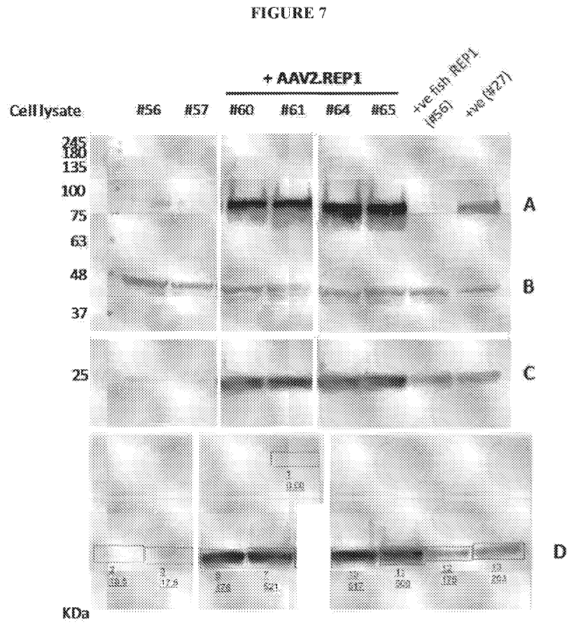

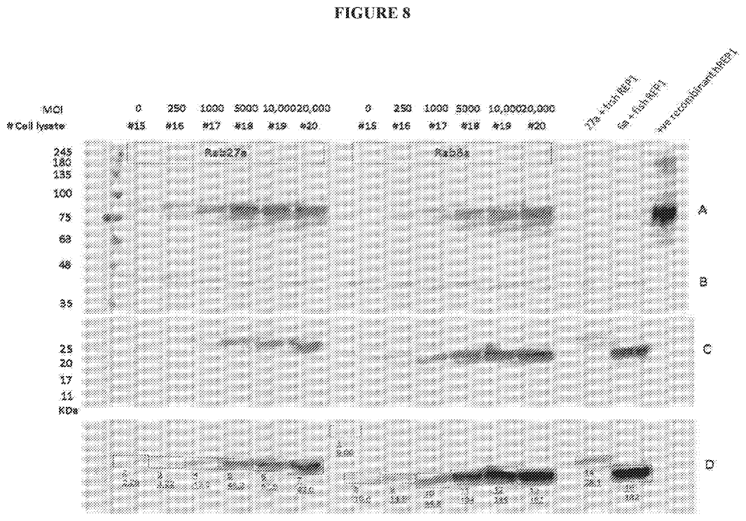

The results indicate that Rab6a works as a substrate for the prenylation assay to assess REP1 function following transduction of HT1080 cells with AAV-REP1 (FIG. 7).

### Example 5

### Comparison of Rab27a and Rab6a as Substrates in Prenylation Reactions

The same cell lysates were used as in the experiment shown in FIG. 4: (a) Untransduced cells; (b) Cells+AAV-REP1 MOI 250; (c) Cells+AAV-REP1 MOI 1,000; (d) Cells+AAV-REP MOI 5,000; (e) Cells+AAV-REP1 MOI 10,000; and (1) Cells+AAV-REP1 MOI 20,000.

Prenylation reactions were set up using 20 μg of lysate in a total volume of 15 μL, and 2 different substrates: Rab27a and Rab6a. Positive controls, one for each substrate, were spiked with 0.1 μM of fish REP1. Samples were run in parallel on SDS-PAGE and detected simultaneously.

Both Rab27a and Rab6a work as a substrate for the prenylation assay to assess REP1 function following transduction of cells with AAV-REP1.

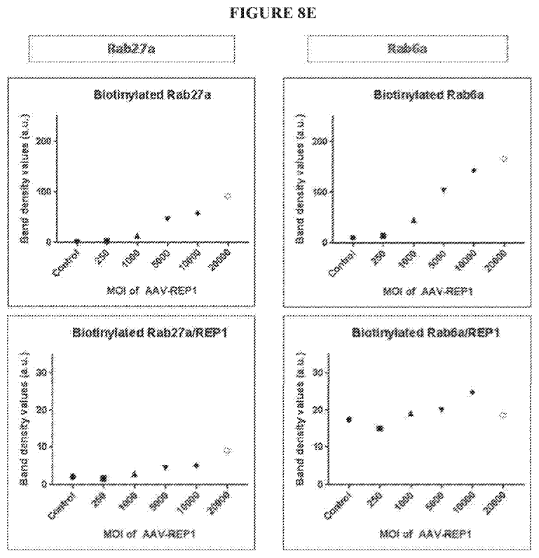

The incorporation of biotinylated lipid donor correlates with the amount of AAV-REP1 used for cell transduction for each of the substrates used (FIG. 8).

The band density from biotinylated Rab6a is higher than for Rab27a, which indicates Rab6a is a more suitable substrate for a parallel line analysis for determination of relative potency and/or biological activity.

### Example 6

### Comparison of Rab27a and Rab6a Performance as Substrates in Prenylation Reactions Using Different Conditions

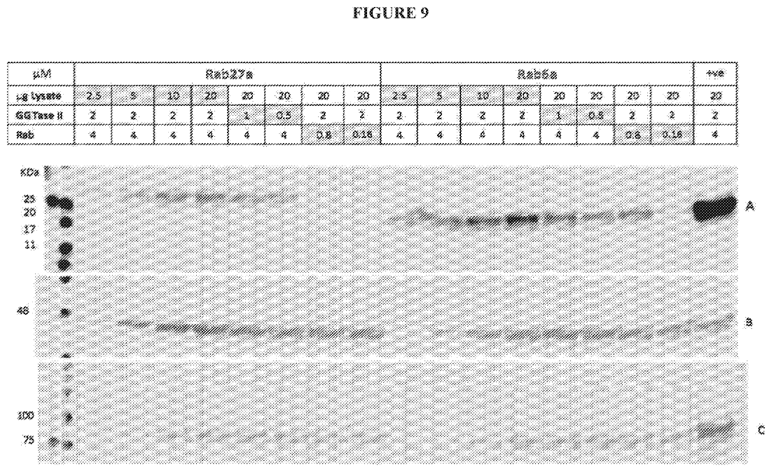

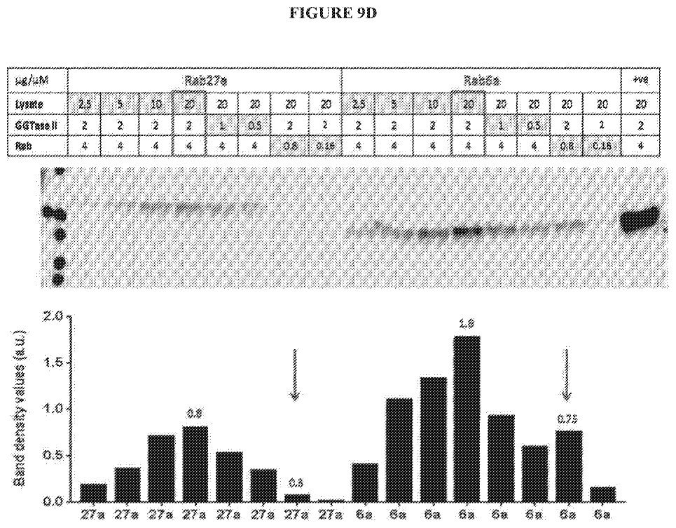

Untransduced lysate of 293 cells was prepared for use in this experiment using Rab27a and Rab6a. The conditions tested are shown in the tables in FIG. 9. Samples were run in parallel on SDS-PAGE and detected simultaneously.

Both Rab27a and Rab6a work as a substrate for the prenylation assay to assess endogenous REP1 function.

The incorporation of biotinylated lipid donor correlates with the amount of total protein in the reaction for each of the substrates used.

Comparing the conditions, the concentration of Rab substrate in the reaction seems to affect the signal the most.

There is a 2.5-fold increase in the biotinylated substrate when Rab6a is used, compared to Rab27a.

### Example 7

### Comparison of Rab27a and Rab6a as Substrates in Prenylation Reactions in Lysates Transduced with AAV2-REP1

New lysates (in triplicate) were prepared using increasing MOIs of AAV2-REP1 (R&D material): (a) Untransduced cells; (b) Cells+AAV-REP1 MOI 100; (c) Cells+AAV-REP1 MOI 500; (d) Cells+AAV-REP1 MOI 1,000; (e) Cells+AAV-REP1 MOI 5,000; (f) Cells+AAV-REP1 MOI 10,000; (g) Cells+AAV-REP1 MOI 20,000; and (h) Cells+AAV-REP1 MOI 50,000.

Prenylation reactions were prepared using 20 μg of total protein, 2 μM of Rab substrate (Rab27a or Rab6a) and 2 μM of Rab GGTase, in a total volume of 10 μL. Positive controls, one for each substrate, were spiked with 0.1 μM of fish REP1.

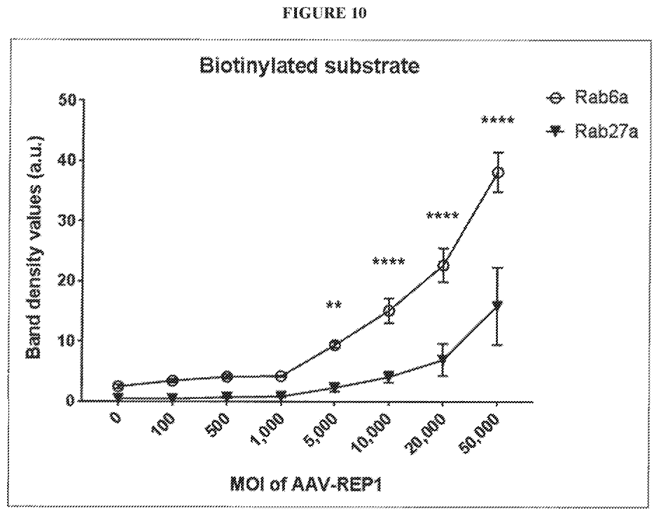

Samples from each replicate were run in parallel on SDS-PAGE and detected for biotinylated substrate (1:10,000), β-actin (1:50,000) as loading control and human REP1 (1:2,500) using image Studio Lite software. Data from semiquantification of band density for biotinylated substrate was plotted using Prism software (FIG. 10).

The levels of β-actin were similar in all samples analyzed. Untransduced cells (and positive control samples) showed endogenous level of REP1. Cells transduced with AAV-REP1 showed an increase of REP1 levels that directly correlates with the MOI used. Positive controls show stronger biotin incorporation, as a result of fish REP1 activity.

A two-way ANOVA analysis of all three replicates with substrate and MOI as factors found that both were highly significant (p<0.0001). Bonferroni's multiple comparisons test for the effect of the substrate at a given MOI showed a significant pairwise difference at MOI of 5,000 (p=0.0023) and all above (p<0.0001).

Both Rab27a and Rab6a work as a substrate for the prenylation assay to assess REP1 function following transduction of cells with AAV-REP1.

Semiquantification of band density for biotinylated substrate only shows the values for Rab6a to be significantly higher than those obtained for Rab27a.

### Example 8

### Prenylation of Rab6a as a Biological Activity for Choroideremia Gene Therapy

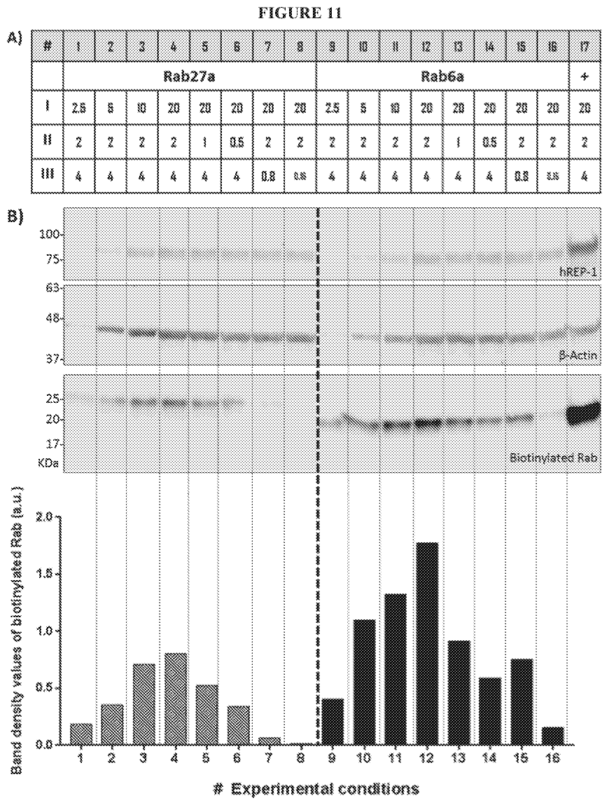

Protein incorporation of biotin-containing isoprenoids (biotin-labelled geranyl pyrophosphate, B-GPP) was used to detect prenylated proteins due to their superior sensitivity relatively to fluorescence-based methods. The first step in establishing an assay of this nature was to optimize the prenylation reaction conditions to detect endogenous REP1 activity (FIG. 11). All reactions were run in parallel using the same cell lysate. Initially different amounts of total cell lysate from 293 cells (also known as HEK293 or human embryonic kidney 293) were tested (2.5 μg, 5 μg, 10 μg and 20 μg) while concentrations of GGT-II (2 μM) and Rab substrate (4 μM, Rab27a or Rab6a) were kept fixed. Lower concentrations of GGT-II (1 and 0.5 μM) and substrate (0.8 and 0.16 μM) were then tested using 20 μg of total cell lysate (FIG. 11A). Both substrates were prenylated in vitro by endogenous REP1 in a dose-dependent manner (top panel of FIG. 11B, conditions 1-4 and 9-12), suggesting both could be used to assess the biological activity of AAV2-delivered REP1. Moreover, when the amount of total protein was kept the same, both the concentration of GGT-II, as well as the concentration of the substrate affected the biotin incorporation in a dose-dependent manner (bottom panel in FIG. 11B, conditions 5-8 and 13-16). The signal obtained with Rab6a was consistently higher than with Rab27a in all otherwise matching conditions tested as measured by the band density values (FIG. 11C). This difference could be as high as 2.5-fold (0.8 versus 1.8 in conditions 4 and 12; 0.3 versus 0.75 in conditions 7 and 15).

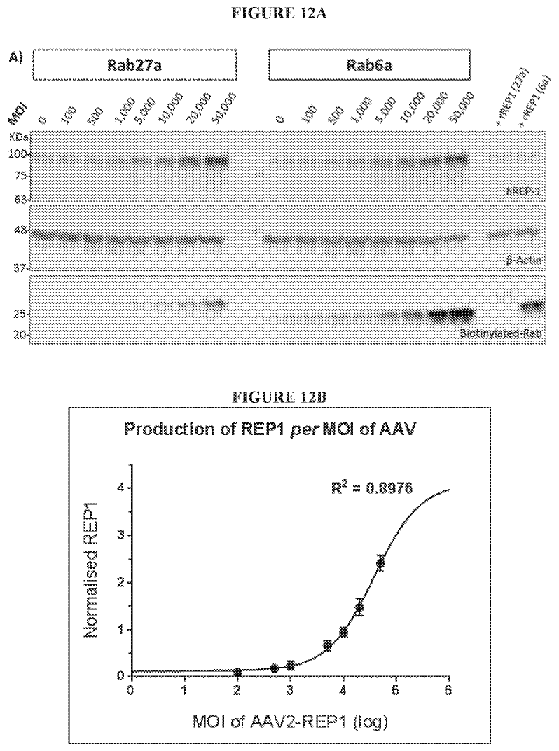

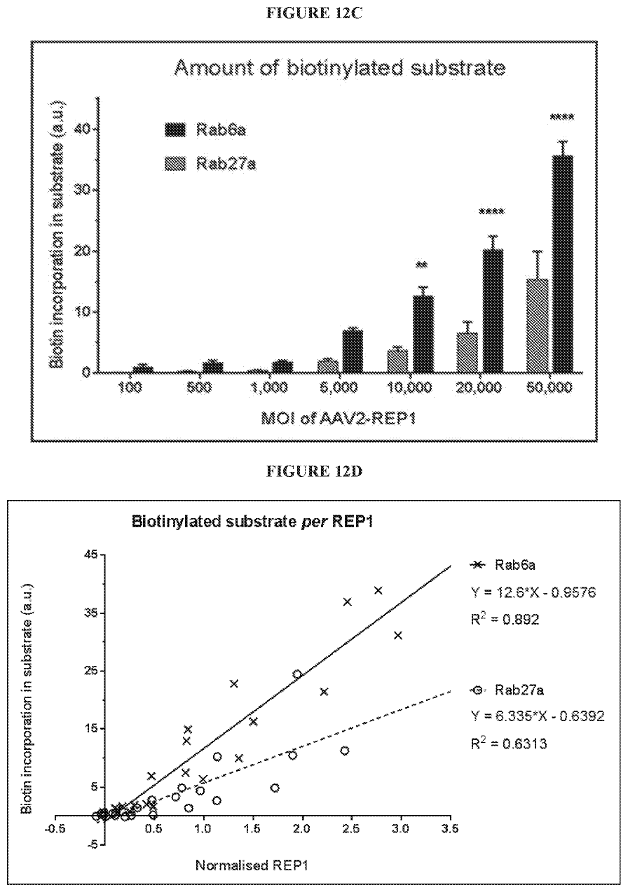

Next, 293 cells were transduced with AAV2-REP1 at a range of multiplicities of infection (MOI, defined as number of genome copies/cell or gc/cell). Prenylation reactions were run with both substrates in parallel (FIG. 12A) to test both Rah proteins as substrates in a scenario of CHM gene augmentation. Given the nonlinear relationship between REP1 expression and MOI of AAV2-REP1, the model used for curve-fitting analysis was the 4-parameter logistic (4-PL) regression model (FIG. 12B), The log (IC₅₀) to be 4.578 (IC₅₀=37,887 MOI), i.e. the MOI that gives a response half way between the basal response and the maximal response is ˜38,000. The amount of biotinylated substrate as measured by the biotin incorporation was plotted against MOI of AAV2-REP1 (FIG. 12C). A two-way ANOVA with substrate and MOI as factors revealed both factors were significant (n=3, p<0.0001). A Bonferroni's multiple comparisons test for biotin incorporation relative to untransduced cells (MOI=0) for each substrate found it to he statistically significant in Rab27a at MOI 20,000 (p=0.0329) and 50,000 (p<0.0001). For Rab6a the biotin incorporation over untransduced cells was found to be statistically significant at MOI 5,000 (p=0,0196) and above (p<0.0001). Finally, the relationship between incorporation of biotin in each substrate against REP1 was plotted in FIG. 12D. Both parameters were corrected for endogenous levels present in untransduced cells. A linear regression analysis on both data sets showed that incorporation of biotin on Rab6a per unit of normalized overexpressed REP1 is consistently higher than for Rab27a. The Rab6a data set also showed a better fit to the regression (R²=0.892 versus R²=0.6313 for Rab27a). Altogether, the data show that Rab6a is more sensitive to use as a substrate to measure changes in prenylation activity.

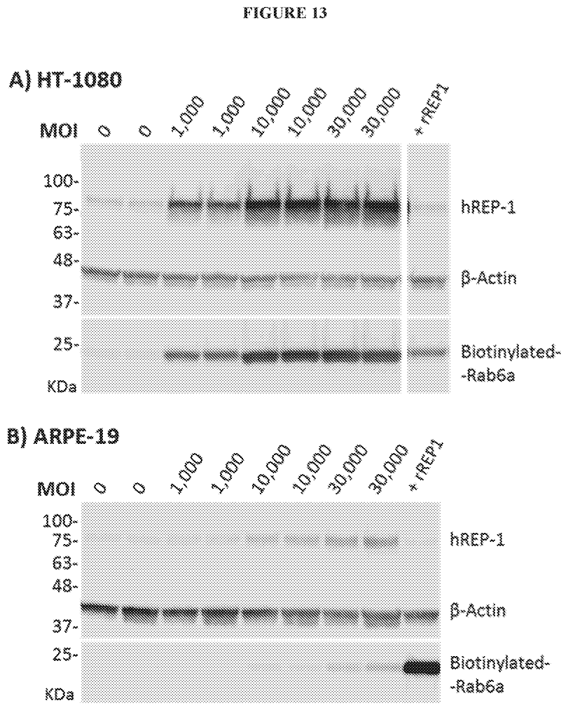

To assess the use of Rab6a as a substrate in an in vitro prenylation assay, other cell lines were transduced in a similar manner. Prenylation reactions were prepared using Rab6a as a substrate, and the obtained results are depicted in FIG. 3. Both HT-1080 (human fibrosarcoma) and ARPE-19 (human RPE) cell lines were transduced at MOI 1,000, 10,000 and 30,000 (FIGS. 13A and 13B, respectively). In both cases the level of biotinylated substrate is proportional to the amount of REP1, showing that our method can be reproduced in other cell lines.

Unless a cell or cell line is modified to become a REP1 deficient cell or cell line, an endogenous level of REP1 is present in the cell or cell line. Thus, an assay which maximizes the measured response provides a superior property by distinguishing between the endogenous and the vector transgene expressed protein.

This study demonstrates the use of a biotinylated lipid donor and a Rab substrate to measure the biological activity of AAV2-delivered REP1 in vitro. The assay described herein provides a sensitive and reproducible in vitro test for assessing the biological activity of AAV gene therapy vectors.

Rab6a is at the exact opposite of Rab27a regarding the prenylation rate: it is at the top hierarchy of Rab proteins prenylation rate and will therefore provide a more sensitive readout of increased activity. Thus, the present disclosure compares Rab6a with Rab27a for use as a substrate in a biological activity assay. The data show that both substrates could be used to measure prenylation activity in untransduced cells. Both substrates were tested to determine how each substrate would behave in response to AAV2-delivered REP1. The relationship between REP1 expression and MOI is not linear but rather logarithmic. The sigmoidal-shaped curve implies there may be a limit for the amount of REP1 expressed from an exogenously-delivered transgene that can be measured using this protocol, which we have not reached in this experiment. The linear regression analysis run on both data sets shows that Rab6a has a higher biotin incorporation (FIG. 12D) within the range where normalized REP1 is linear (˜1 to ˜2 log gc/cell in FIG. 12B). Therefore, Rab6a is the substrate that predicts more accurately how much biotin is incorporated per unit of overexpressed REP1.

The use of Rab6a was further validated in other cell lines. HT-1080 cells have been used before to test a lentiviral construct delivering REP1 and to confirm REP1 expression following the use of AAV2-REP1 in a choroideremia gene therapy trial (NCT01461213). ARPE-19 cells were selected for their similarity to the target cell type of choroideremia gene therapy. Both cell lines responded as 293 cells regarding the incorporation of biotin in Rab6a following an in vitro prenylation protocol, confirming this assay is reproducible and does not appear to be cell type-specific.

Altogether, the data show that in vitro prenylation of Rab6a is a more sensitive and robust method to test REP1 transgene expressed activity following cell transduction with AAV2.

### Example 9

### Comparison of Rab27a and Rab6a as Substrates in Prenylation Reactions

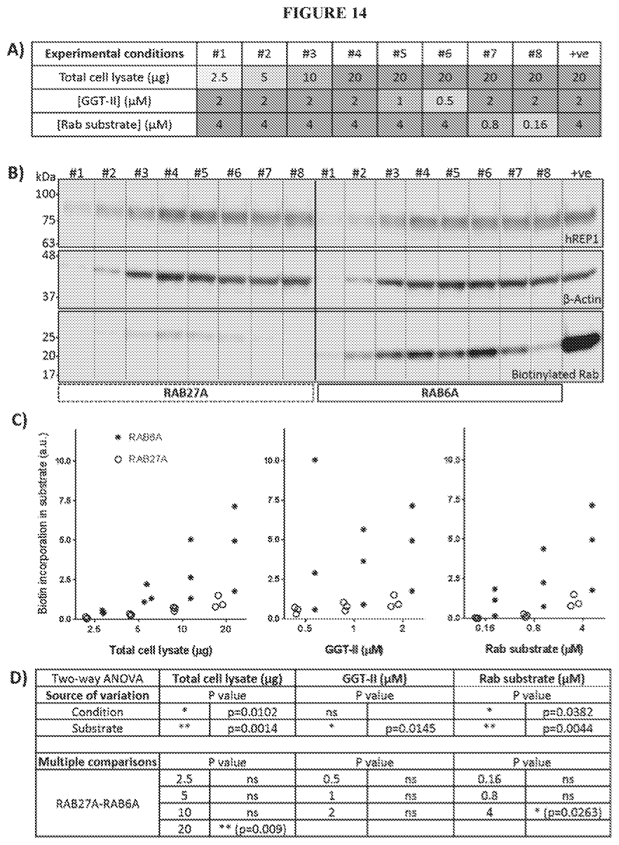

Total cell lysate (20 μg), GGT-II (2 μM) and Rab substrate (4 μM) were used as standard conditions in investigating differences in biotin incorporation in RAB27A and RAB6A using 293 cells. The first step in establishing this assay was to optimize the prenylation reaction conditions to detect endogenous REP1 activity (FIG. 14). The experimental conditions tested are depicted in FIG. 14A, and include amount of total cell protein (2.5, 5, 10 and 20 μg), concentration of GGT-II (0.5, 1 and 2 μM) and concentration of Rab substrate, either RAB27A or RAB6A (0.16, 0.8 and 4 μM). Three separate cell lysates were used to run the three independent experiments. The reaction products were subjected to western blot analysis, of which one representative in shown in FIG. 14B. The positive control (+ve) reaction was run with 2 μM of GGT-II and 4 μM of RAB6A, and spiked with recombinant fish REP1 (25 nM). The band intensity for biotin incorporation in the positive control well (FIG. 14B, right hand side) is proof that all substrates involved in the reaction were in appropriate conditions. In all three experiments it was observed that both substrates were prenylated in vitro by endogenous REP1 in a dose-dependent manner as measured by the biotin incorporation (FIGS. 14B and 14C). Both can be used to assess the biological activity of rAAV2/2-delivered REP1. As for statistical data analysis, three independent two-way ANOVA were run to compare the biotin incorporation between RAB27A and RAB6A for each of the conditions tested. The two-way ANOVA with ‘condition’ (total cell lysate) and ‘substrate’ as factors revealed both factors were significantly contributing to the source of variation in conditions #1-#4 (FIG. 14D; n=3; p=0.0102 and p=0.0014, respectively). However, a Bonferroni's multiple comparison test for biotin incorporation found RAB6A to have incorporated significantly more B-GPP than RAB27A when 20 μg of total cell lysate were used (FIG. 14D; p=0.009). The same approach was used for analyzing the impact of both concentration of GGT-II (conditions #4-#6) and concentration of Rab substrate (conditions #4, #7 and #8). The two-way ANOVA analysis with GGT-II concentration as ‘condition’ revealed that only the ‘substrate’ contributes to the source of variation in this case (FIG. 14D; n=3; p=0.0145). The Bonferroni's multiple comparison tests for biotin incorporation found no statistically significant differences between RAB27A and RAB6A when the concentration of GGT-II varied (FIG. 14D; ns). Regarding the Rab substrate concentrations (conditions #4, #7 and #8), a two-way ANOVA analysis showed both ‘condition’ and ‘substrate’ to be contributing factors to the source of variation (FIG. 14D; n=3; p=0.0382 and p=0.0044, respectively). A Bonferroni's multiple comparison test for biotin incorporation found RAB6A to have incorporated significantly more B-GPP than RAB27A when 4 μM of Rab substrate were used (p=0.0263). These data shows that different Rab substrates influenced the results obtained in all conditions tested. Moreover, the concentration of GGT-II in reaction is the least contributing factor for the biotin incorporation in the substrate, possibly because it was used in excess.

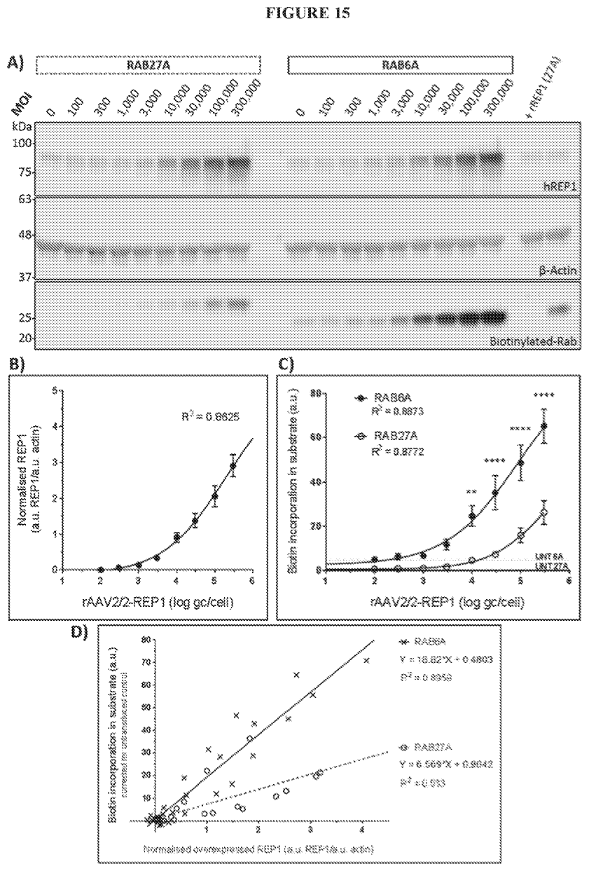

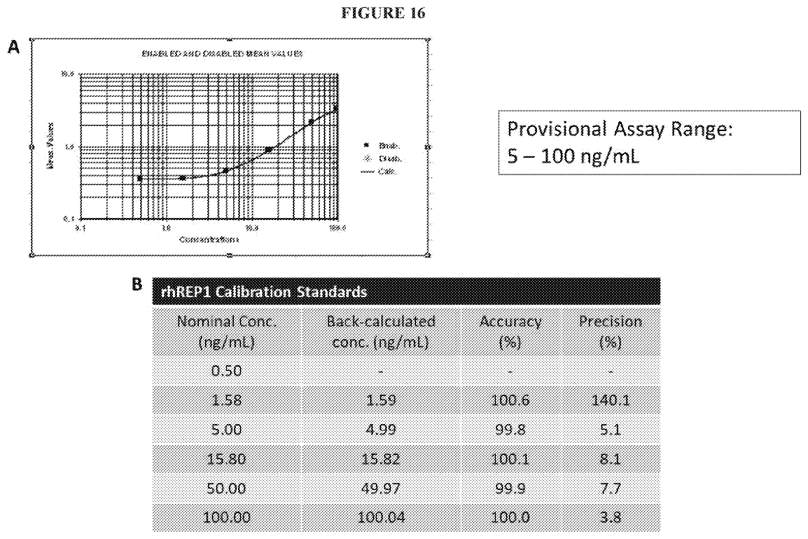

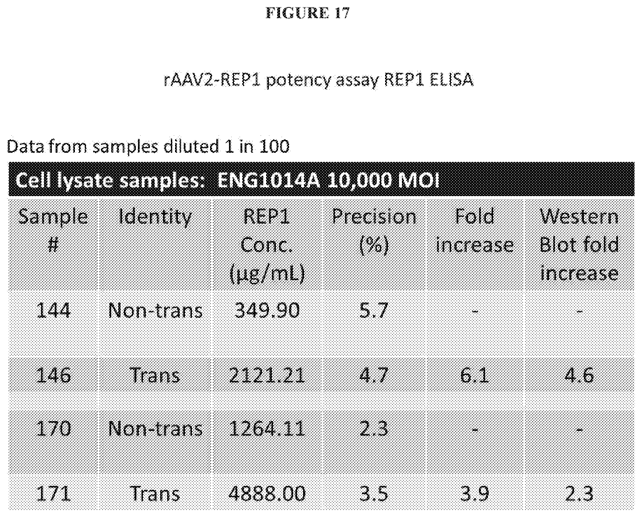

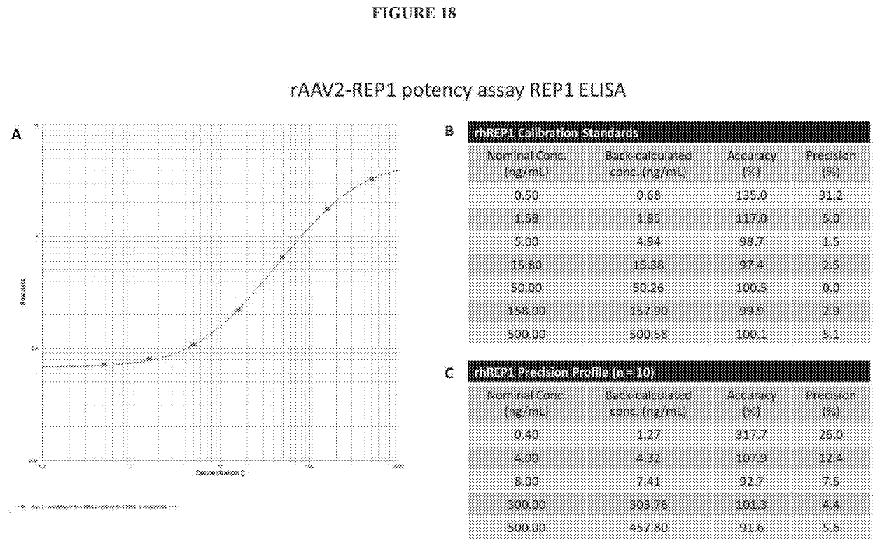

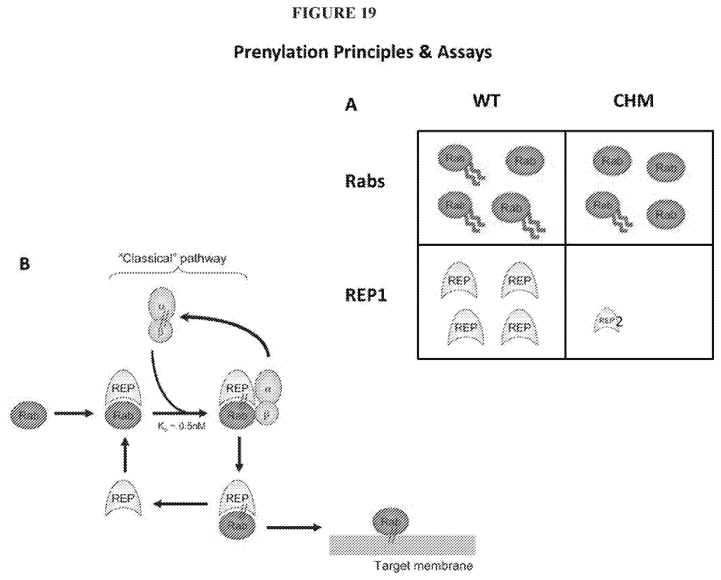

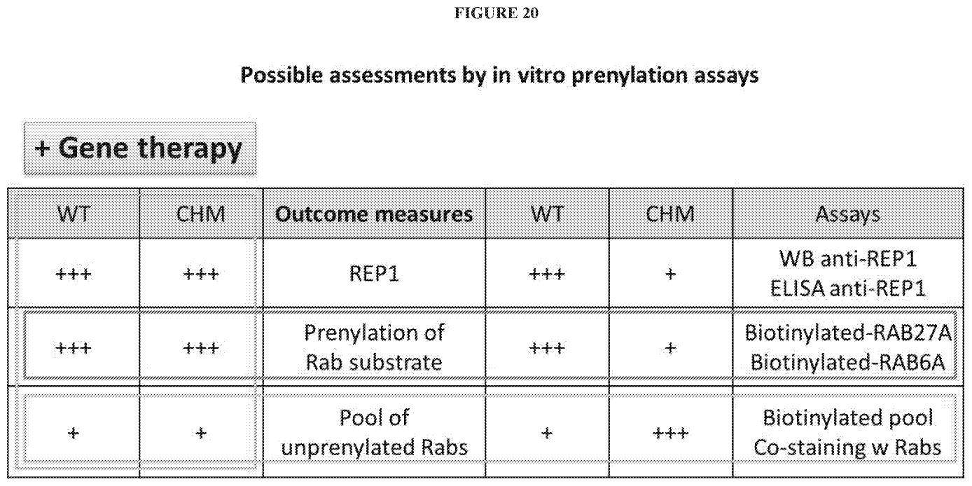

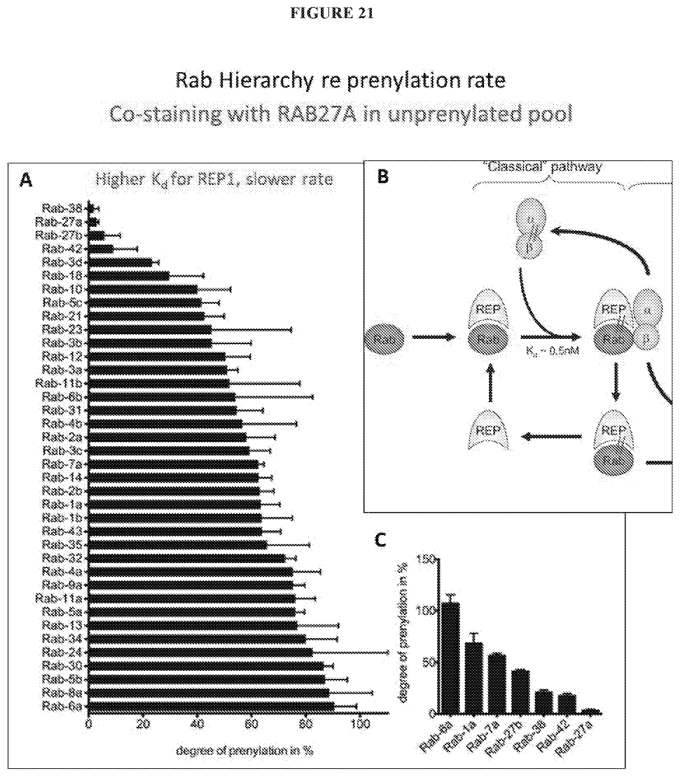

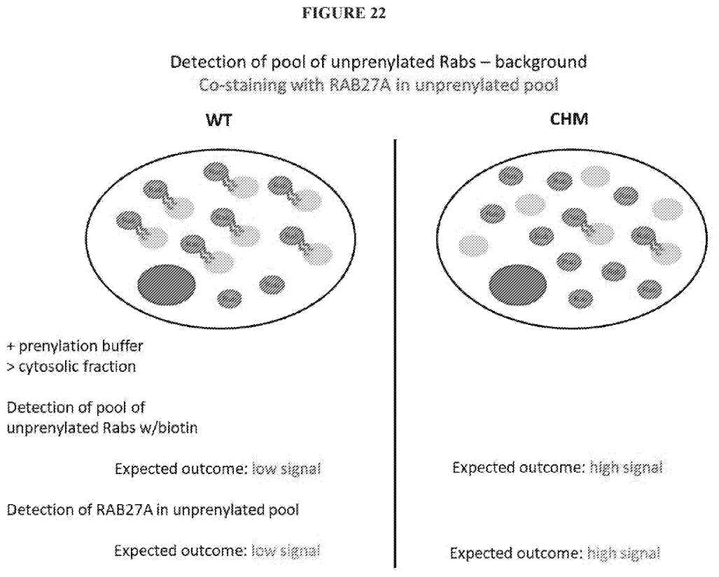

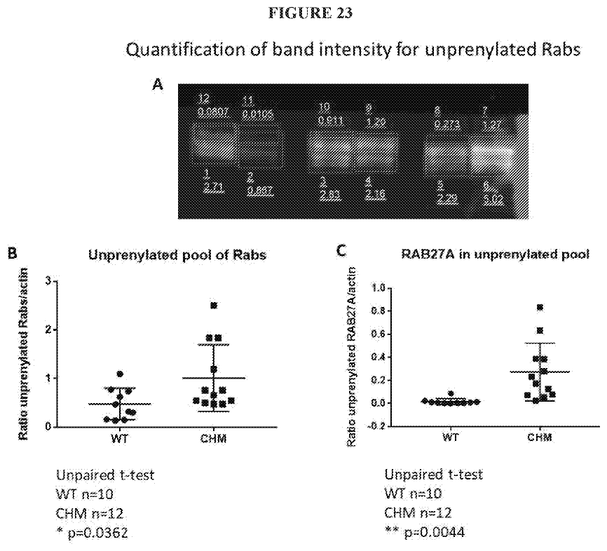

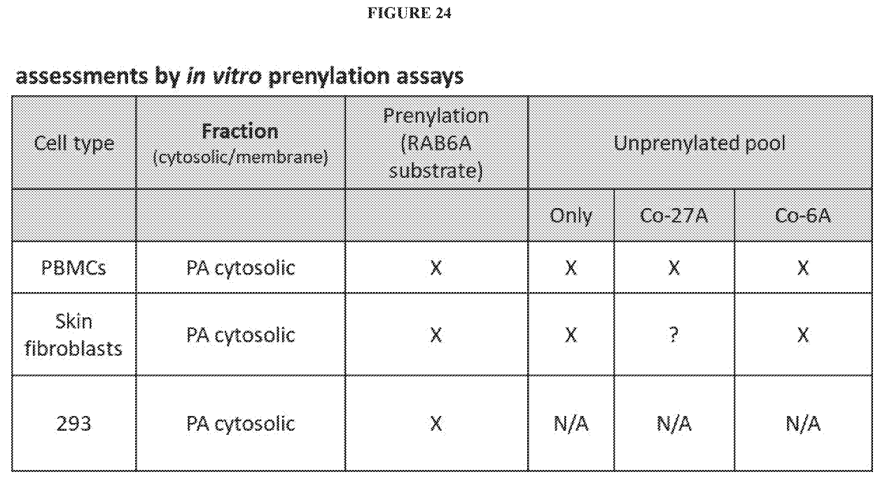

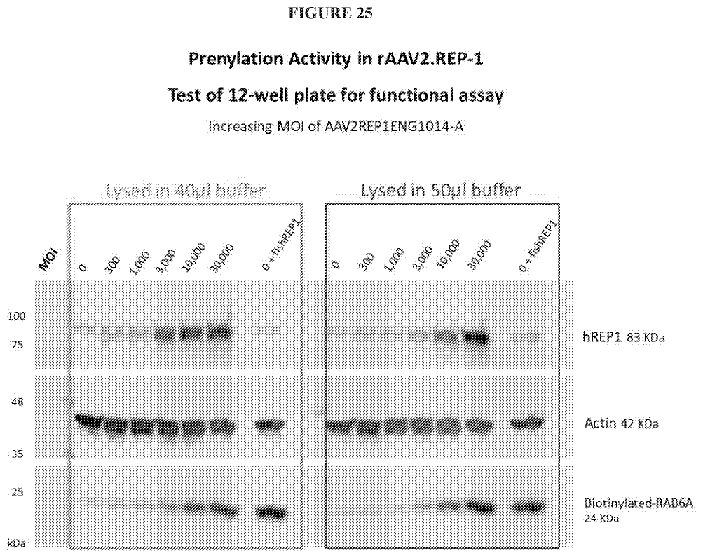

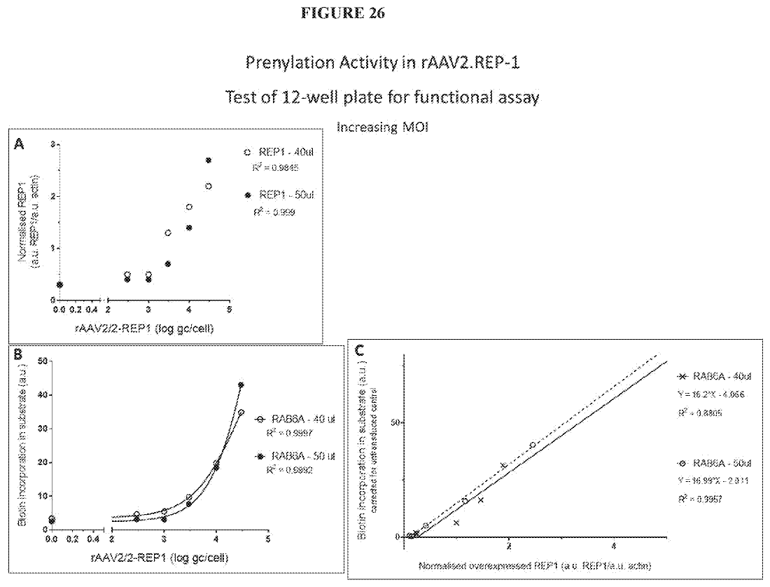

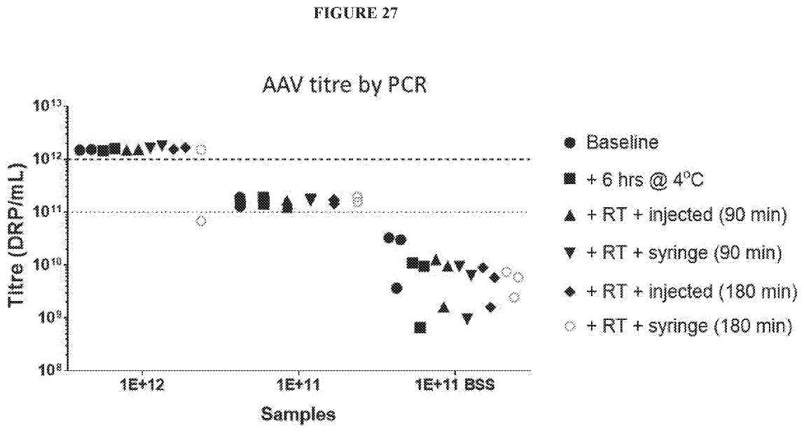

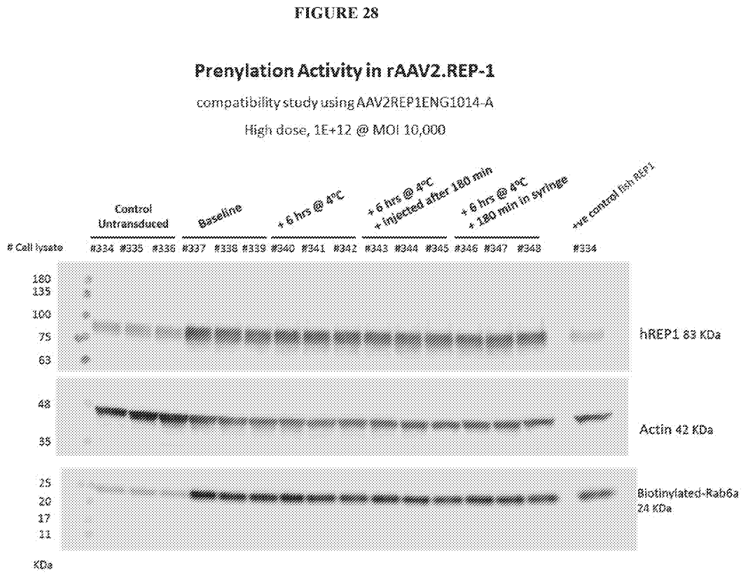

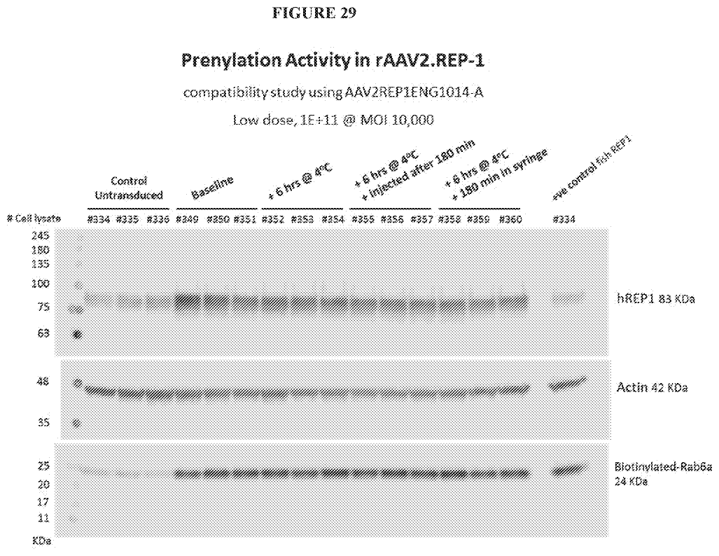

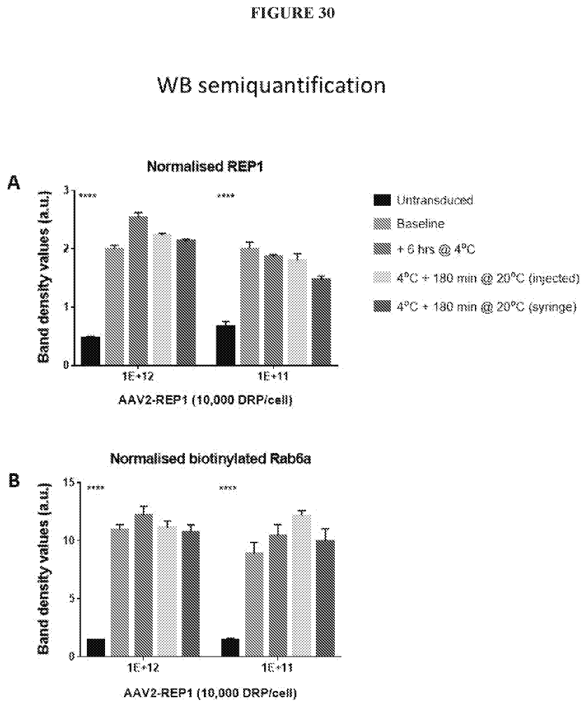

Both Rab proteins were tested as substrates in a scenario of CHM gene augmentation. Three independent experiments were run where 293 cells were transduced with rAAV2/2-REP1 at a range of increasing multiplicities of infection (MOI, defined as number of genome copies/cell or gc/cell) (100; 300; 1,000; 3,000; 10,000; 30,000; 100,000; 300,000) (FIG. 15), The prenylation reaction products were analyzed simultaneously in each experiment, using actin as a loading control; a representative western blot is shown in FIG. 15A. Two positive control reactions (one for each Rab substrate) were run in parallel with recombinant fish REP1 (25 nM) spiked in the untransduced cell lysate.

It was observed that the amount of REP1 detected by western blot correlates to the amount of viral particles added to the cells (FIG. 15A, top panel): the band density for REP1 increases as the MOI increases. Normalized REP1 band density (to corresponding actin band density) was plotted against the MOI (log scale) using a 4-parameter logistic (4-PL) regression model (FIG. 15B). This model took into consideration the fact that cells were a biologically limited system in this experiment, where increasing MOI will saturate the system at some point and cease REP1 production. The regression model was run with no constrains (R²=0.8625), and predicted the best-fit value for the top of the curve to be 5.191 arbitrary units (a.u.) of normalized REP1. The log(IC₅₀) for this fit was 5.255 a.u., corresponding to a MOI of 179,735 gc/cell, which is within the range that was tested.

Regarding biotin incorporation in the Rab substrate (FIG. 15A, bottom panel), it was further observed that the incorporation of biotin in RAB6A was detected over a wider range than in RAB27A. Following the same rationale as for REP1 in FIG. 15B, the biotin incorporation in both substrates (as measured by the band density) was plotted against the MOI of rAAV2/2-REP1 used for transduction (FIG. 15C). The baseline value obtained for each Rab in the untransduced samples (average of three independent runs) was represented by the horizontal dotted line (RAB6A, 5.009±1.25 a.u.; RAB27A, 0.577±0.19 a.u.). A 4-PL regression model was run for each Rab substrate, without any constrains, and both R² are shown in FIG. 15C (RAB27A, R²=0.8772; RAB6A, R²=0.8873). The best-fit prediction for the RAB6A top of the curve was 92.83 a.u., with a log(IC₅₀) of 4.912, corresponding to a MOI of 81,694 gc/cell. For RAB27A, the top of curve was predicted to be 53.8 a.u., with a log(IC₅₀) of 5.514, corresponding to a MOI of 326,488 gc/cell. The differences between the log(IC₅₀) values for each Rab substrate were indicative of their sensitive in this assay: incorporation of biotin in RAB6A can be detected over a wider range than in RAB27A, which displays a lower slope and limit of detection. These findings were reinforced by the two-way ANOVA run in the same data set, with ‘MOI’ and ‘substrate’ as factors: both were found to be significant (n=3; p<0.0001). Moreover, Bonferroni's multiple comparison tests for biotin incorporation in substrate at each tested MOI revealed that such incorporation was significantly higher in RAB6A than RAB27A at the MOI of 10,000 (p=0.0097), 30,000 (p=0.0002) and 100,000 and 300,000 (p<0.0001), RAB6A was superior in incorporating biotin at a given MOI of rAAV2/2-REP1.

RAB6A was more sensitive to use as a substrate to measure the biological activity of rAAV2/2-REP1. Each value of the biotin incorporation in substrate was plotted, corrected for the corresponding untransduced sample, against the normalized overexpressed REP1 (FIG. 15D). The resultant linear regression analysis showed that incorporation of biotin on RAB6A per unit of REP1 was higher than for RAB27A (Y=18.82*X+0.4803 versus Y=6.569*X+0.9042, respectively). The RAB6A data set also showed a better fit to the regression (R²=0.8959 versus R²=0.533 for RAB27A).

The use of RAB6A as a substrate in an in vitro prenylation assay was confirmed in other cell lines. The cell lines HT-1080 (human fibrosarcoma) and ARPE-19 (human RPE) were transduced with rAAV2/2-REP1 in a similar manner for a qualitative analysis. In both cases, the representative MOI of 1,000, 10,000 and 30,000 gc/cell were used to transduce two wells (replicates) in one single experiment A positive control was run in parallel with recombinant fish REP1 spiked in each untransduced cell lysate (25 nM for HT-1080; 11 nM for ARPE-19). The prenylation products were subjected to western blot analysis and the results are shown in FIG. 13. We observed a correlation between the MOI used for transduction, the expression of REP1 and the incorporation of biotin in RAB6A, as we did for 293 cells (FIGS. 13A and 13B). However REP1 levels detected for ARPE-19 and corresponding biotinylated-RAB6A were overall lower than for HT-1080 and 293 cells. ARPE-19 cells are larger in size, which required a reduced number of cells seeded in each well, and volume restrictions to the total amount of cell lysate that could be loaded in the gel.

The disclosure reports for the first time the use of a biotinylated lipid donor and a Rab substrate to measure the biological activity of rAAV2/2-delivered REP1 in vitro. The aim is to provide a reproducible and sensitive in vitro test for assessing the biological activity of rAAV gene therapy vectors for choroideremia.

Underprenylation of RAB27A is one of the molecular causes of degeneration of RPE cells in choroideremia, although other cellular perturbations may contribute to the choroideremia phenotype. For example, RAB27A is among a subset of Rab proteins that are under-prenylated in choroideremia lymphoblasts. RAB27A has a lower affinity for REP2 than for REP1 than other Rab proteins, although RAB27A binds equally well to REP1 and REP2. RAB27A may accumulate unprenylated due to the fact that the RAB27A -REP1 complex has a higher affinity for GGT-II than RAB27A -REP2. Furthermore, RAB27A has both one of the slowest rates of GTP hydrolysis and one of the slowest prenylation rates among Rab proteins.

Biotinylated lipid donors are beneficial in biological assays. As defined by the US Food and Drug Administration (FDA), a biological assay is a “quantitative assay that measures the activity of the product related to its specific ability to effect a given result”. Simple and sensitive methods of assessing prenylation in vitro are possible using biotinylated lipid donors. Unprenylated Rab protein levels have been detected using biotin-labelled prenyl donors in HeLa, lymphoblasts, fibroblasts and iPS-derived RPE cells.

The RAB6A substrate predicts more accurately how much biotin is incorporated per unit of overexpressed REP1 than RAB27A. HEK293 cells were the cell line of choice for this study. HEK293 cells have characteristics for the development a potency test for gene therapy products according to the FDA recommendations. HEK293 cells commercially available from a certified cell line provider and as a master cell bank compliant with current Good Manufacturing Processes (cGMP). Moreover, due to REP1 ubiquitous expression, and in the absence of a REP1-deficient stable cell line, there will always be an endogenous level of REP1 present. Therefore, an assay which maximizes the measured response is the most beneficial to distinguish between the endogenous and the vector transgene-expressed REP1. RAB27A was compared with RAB6A. RAB6A is at the exact opposite of RAB27A regarding the prenylation rate: it is at the top hierarchy of Rab proteins prenylation rate. RAB6A provided a more sensitive readout of biological activity. RAB6A was compared with RAB27A for use as a substrate in a biological activity assay. Both substrates could be used to measure prenylation activity in 293 untransduced cells. The band density obtained with RAB6A was constantly higher than RAB27A. Both substrates were tested for how they would behave in response to rAAV2/2-delivered REP1. The expression of REP1 is proportional to the amount of rAAV2/2-REP1 used to transduce the cells. The statistical best-fit for the relationship between REP1 expression and MOI is not linear but rather logarithmic, due to the cells being a system where there is a limit to the amount of rAAV that could transduce it. The sigmoidal-shaped curve shows there is a limit for the amount of REP1 expressed from an exogenously-delivered transgene that can be measured using this protocol. This limit has not been reached in the experiment of this disclosure. This is also true for biotin incorporation in the Rab substrate. RAB6A is a more efficient substrate to use to measure biotin incorporation, as its range is wider and steeper than RAB27A. The linear regression analysis run on both data sets shows that RAB6A has higher biotin incorporation within the range where normalized REP1 is linear.

The use of RAB6A was further assessed in other cell lines. ARPE-19 cells were selected for their similarity to the target cell type of choroideremia gene therapy. Both cell lines responded as 293 cells regarding the incorporation of biotin in RAB6A following an in vitro prenylation protocol. This assay is reproducible and does not appear to be cell type-specific.

Altogether, our data shows that in vitro prenylation of RAB6A is a robust method to test REP1 activity following cell transduction with rAAV2/2. RAB6A appears to be more sensitive to be used as a substrate in a potency assay for rAAV2/2-REP1 as it is capable of detecting minor differences between viral vector batches more accurately than RAB27A as a substrate. The disclosure provides valuable improvements to the development of an in vitro prenylation assay to assess the biological activity of AAV vectors in choroideremia gene therapy, including, for example, gene therapy in the context of clinical trials.

### Example 10

### Assessment of Biocompatibility and Stability and Concentration

The biocompatibility and stability of AAV drug products following storage and passage through injection devices for AAV gene therapy was assessed in a setting that mimicked the clinical scenario. Two doses and diluents of rAAV2.REP-1 were tested. Samples were collected and analyzed to determine if there were any losses of vector, either physical loss or loss of the biological function (e.g. REP-1 prenylation activity).

High dose vector at 1E+12 in TMN200 was diluted into TMN200 and Balanced Salt Solution (BSS) using a 10-fold dilution. The baseline sample and 3 independent loaded surgical devices (a 23G needle with a 41G Teflon tip) were kept at 4° C. for 30 minutes, followed by 90 and 180 minutes at room temperature. Samples collected at all time points from injected and ‘syringe’ samples, and qPCR was used to determine the physical titer (DRP/mL). The level of REP-1 protein expression and activity was determined by WB and in vitro prenylation using biotinylated lipid donors at baseline, 30 min at 4° C. and at 180 minutes at room temperature.

Genomic titer analysis was run to ensure good precision between sample replicates. There were significant losses in the genomic titer of samples diluted with BSS, compared to baseline levels, for all time points tested (a 60-70% drop). Therefore, these were excluded from protein analysis. Samples diluted with TMN200 showed no significant difference to baseline for any of the time points. 4° C. and 180 minute samples showed sustained REP-1 expression compared to baseline. Similarly, the level of biotinylated. Rab substrate did not vary from baseline.

Use of TMN200 as a diluent ensured a physical titer of the AAV drug product even at a lower dilution, as well as level of expression and functionality of drug product over a period up to 3.75 hours.

The determination of the physical viral genome titer is part of the characterization of the vector and is a critical step to ensure viral particle potency and safety for delivery during gene therapy. The most prevalent method to determine the AAV titer is quantitative PCR (qPCR). Different variables that can influence the results, such as the conformation of the DNA used as standard or the enzymatic digestion during the sample preparation.

To analyses the influence of the DNA standard conformation, two standard curves were prepared using the supercoiled plasmid and the linearized form. The linearized plasmid was prepared by digestion with HindIII restriction enzyme, visualized by agarose gel electrophoresis and purified using the QIAquick Gel Extraction Kit (Qiagen) following manufacturer's instructions. Seven serial dilutions of each plasmid standard (109-103 copies of plasmid DNA) were used to generate the standard curves. To extract the DNA from purified AAV vectors, two enzymatic methods were used: single digestion with DNase I and double digestion with an additional proteinase K treatment. QPCR was performed with the CFX Connect Real-Time PCR Detection System (BioRad) using primers and Taqman probe specific to the transgene sequence.

The use of supercoiled plasmid as standard significantly increased the titer of the AAV vector, compared to the use of linearized plasmid (p<0.0001, Paired t-test). Based on the data generated, the absolute difference in the titer values is approximately 4.6-times higher using supercoiled standard compared to the linearized standard. No significant difference (p=0.075, Paired t-test) in the titer was found between samples treated with DNase I and the same samples with the additional Proteinase K treatment.

Standard DNA conformation influences absolute quantification by qPCR, giving rise to an overestimated AAV titer when the supercoiled plasmid is used. These results highlight the importance of using linearized plasmids to get reliable and accurate titers of AAV vectors not only for research purposes but also, to ensure the therapy safety and potency in clinical trials.

### Materials and Methods

AAV vector production: An AAV2 viral vector containing the CHM transgene under the control of a CAG promoter was produced following a standard protocol (Zolotukhin, S, et al. (1999). Gene Ther. 6: 973-985) with some modifications. Briefly, HEK293 (293 human embryonic kidney) cells were co-transfected with calcium phosphate and viral particles were purified from the cell lysates using iodixanol discontinuous centrifugation and heparin chromatography. The viral stock was prepared in formulation buffer (20 Tris pH 8.0, 1 mM MgCl₂, 200 mM NaCl, at pH 8 in water for injections) at a concentration of 4.95E+12 DRP/mL.

Cell culture: HEK293 cells (human embryonic kidney, #85120602, Culture Collections, Public Health England, Salisbury, UK) were cultured in MEM culture medium. HT1080 cells (human fibrosarcoma, #85111505, Culture Collections, Public Health England, Salisbury, UK) were cultured in DMEM. ARPE-19 cells (human RPE, #CRL-2302. ATCC via LGC Standards, Middlesex, UK) were cultured in DMEM:F12. MEM culture medium was supplemented with L-glutamine (2 mM). All three culture media were supplemented with penicillin (100 units/mL), streptomycin (100 μg/mL), non-essential amino acids (1%) and 10% fetal bovine serum. Cells were maintained at 37° C. in a 5% CO₂ environment. RPE-J cells (rat retinal pigment epithelium, #CRL-2240, ATCC via LGC Standards, Middlesex, UK) were cultured in DMEM supplemented with L-glutamine (2 mM), penicillin (100 units/mL), streptomycin (100 μg/mL), non-essential amino acids (1%) and 4% fetal bovine serum. Cells were maintained at 34° C. in a 5% CO₂ environment.

Cell transduction and preparation of total cell lysates: For transduction experiments, all cells were seeded in 6-well plates on the day prior to transduction: 293, 9.5E+05 cells/well; HT1080, 4E+05 cells/well; ARPE-19, 2E+05 cells/well. Transduction with rAAV2/2 was performed at a range of multiplicities of infection (MOI, i.e. genome particles/cell), and media changed 3 days post-transduction (dpt) and every 2-3 days thereafter. Cell lysates were prepared at 5 dpt as follows: cells were washed with PBS and incubated with prenylation buffer (50 mM HEPES, 50 mM NaCl, 2 mM MgCl₂, 1 mM DTT, pH 7.5) supplemented with protease inhibitors (cOmplete™ Mini, Roche, Welwyn, UK) on ice. Cells were scraped into a 1.5 mL tube using and a cell scraper, incubated on ice for 15 min and then disrupted by passing them 20 times through a 26-G needle attached to a 1 mL syringe. Cells were centrifuged for 5 min at 1,500 RCF at 4° C., and the supernatant was transferred to cellulose propionate tubes and centrifuged at 100,000 RCF for 1 h at 4° C. The supernatant was kept as total cell lysate for prenylation reactions in vitro. Total protein content was determined using the Bradford method according to the manufacturer's instructions (Quick Start™ Bradford 1× Dye Reagent, Bio-Rad, Hertfordshire, UK) and samples values were extrapolated from a standard curve using a sigmoidal 4-parameter logistic regression.

In vitro prenylation assay: The prenylation reactions were set up using total cell lysate (up to 20 μg), recombinant rat Rab GGTase (2 μM, Jena Biosciences, Jena, Germany), recombinant human Rab protein (Rab27A, Abnova Corporation, UK; Rab6A, Jena Biosciences, Jena, Germany) and biotin-labelled geranyl pyrophosphate (B-GPP, 5 μM, Jena Biosciences, Jena, Germany) as lipid donor, in prenylation buffer. All reactions were supplemented with fresh guanosine 5′-diphosphate (GDP, 20 μM, Merck Millipore, Watford, UK) and DTT (1 mM, Thermo-Fisher Scientific, Loughborough, UK). Positive controls were prepared using untransduced cell lysate spiked with a recombinant REP1 protein (fish His-REP1, Jena Biosciences, Jena, Germany, or human His-REP1, Nightstar Therapeutics Ltd., UK). The reactions were incubated for 2 h at 37° C. and then stopped by addition of Laemmli sample buffer.

Western blot analysis: Reaction products were subjected to SDS-PAGE on 10% pre-cast polyacrylamide gel (Criterion™, Bio-Rad, Hertfordshire, UK), transferred to a PVDF membrane (TransBlot Turbo, Bio-Rad, Hertfordshire, UK) and blocked with blocking buffer [PBS+0.1% Tween20 (PBST)+3% bovine serum albumin (BSA)] for 45 min. For protein expression, membranes were incubated separately for anti-β-actin (AM4302, Thermo-Fisher Scientific, Loughborough, UK; 1:50,000) and anti-human REP1 (MABN52, Merck Millipore, Watford, UK; 1:2,500) primary antibodies in blocking buffer for 1 hour under agitation. Membranes were washed 3×7 min with PBST, incubated with HRP-labelled secondary antibody for 30 min in blocking buffer (1:10,000), washed again as before, and detected using Clarity ECL (Bio-Rad, Hertfordshire, UK) and an Odyssey Imaging System (LI-COR Biosciences, Cambridge, UK). The incorporation of biotinylated lipid donor into the appropriate Rab substrate was detected by direct incubation with streptavidin-HRP (Thermo-Fisher Scientific, Loughborough, UK) for 30 min. Densitometry data analysis was performed using the ImageStudio Lite software (LI-COR Biosciences, Cambridge, UK).

Statistical analysis: REP1 expression levels were normalized to actin as loading control. The normalized REP1 was plotted against log-base-10 transformed MOI of AAV2-REP1 and fitted to a four-parameter logistic (4-PL) regression model with 95% confidence interval (CI), hill slope=1 and bottom>0 constrain (mean of 6 replicates±SEM). Biotin incorporation for both substrates for each MOI was compared using a two-way analysis of variance (ANOVA) with substrate and MOI as factors of 3 replicates±SEM). The Bonferroni test was applied to correct for multiple comparisons (95% CI). Biotin incorporation in each substrate was plotted against the levels of normalized REP1 (corrected for untransduced control sample) and analyzed by linear regression (95% CI). All statistical analysis was done using Prism 7 for Windows (San Diego, Calif., USA).

For FIGS. 14 and 15, biotin incorporation in both RAB27A and RAB6A using different experimental conditions was compared using a two-way ANOVA with ‘substrate’ and ‘condition’ as factors (mean of 3 replicates÷SEM). The Bonferroni test was applied to correct for multiple comparisons, with a 95% confidence interval (CI). The normalized REP1 (corrected for corresponding actin levels) was plotted against log-base-10 transformed MOI of rAAV2/2-REP1 (log gc/cell) and fitted to a four-parameter logistic (4-PL) regression model with 95% CI, no constrains (mean of 6 replicates±SEM). Biotin incorporation in both substrates was plotted against the MOI of rAAV2/2-REP1 (log gc/cell) and fitted to a 4-PL regression model with 95% CI, no constrains (mean of 3 replicates±SEM). Biotin incorporation per substrate for each MOI was compared using a two-way ANOVA with ‘substrate’ and ‘MOI’ as factors. The Bonferroni test was applied to correct for multiple comparisons (95% CI). Biotin incorporation in each substrate was plotted against the levels of normalized REP1 (corrected for untransduced control sample) and analyzed by linear regression (95% CI). All statistical analysis was done using Prism 7 for Windows (San Diego, Calif., USA),

### INCORPORATION BY REFERENCE

All publications mentioned in the above specification are herein incorporated by reference. Various modifications and variations of the described methods and uses of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention, which are obvious to those skilled in biochemistry and biotechnology or related fields, are intended to be within the scope of the following claims.

### Other Embodiments

While particular embodiments of the disclosure have been illustrated and described, various other changes and modifications can be made without departing from the spirit and scope of the disclosure. The scope of the appended claims includes all such changes and modifications that are within the scope of this disclosure.

What is claimed is:

1. A method for determining an activity of Rab escort protein 1 (REP1) comprising the steps:
(a) contacting a REP1 protein with a Rab6a protein, a Rab geranylgeranyltransferase (Rab GGTase) and a lipid donor substrate to produce a lipidated Rab6a; and
(b) detecting the lipidated Rab6a.

2. The method of claim 1, wherein a sample comprises the REP1 protein.

3. The method of claim 2, wherein the sample comprising the REP1 protein is isolated or derived from a cell and wherein the cell is genetically engineered to express the REP1 protein.

4. The method of claim 3, wherein the sample comprising the REP1 protein comprises a lysate of the cell.

5. The method of any one of claims 1-4, wherein the REP1 protein is expressed from a viral vector comprising a nucleotide sequence encoding the REP1 protein.

6. The method of claim 5, wherein the viral vector is an adeno-associated viral (AAV) vector.

7. The method of any one of claims 1-6, wherein the Rab6a protein or the Rab GGTase is substantially pure.

8. The method of any one of claims 1-6, wherein the Rab6a protein and the Rab GGTase are substantially pure.

9. The method of any one of claims 1-8, wherein the Rab6a:Rab GGTase molar ratio is about 1:2-3.

10. The method of any one of claims 1-8, wherein the Rab6a:Rab GGTase molar ratio is 1:2-3.

11. The method of any one of claims 1-8, wherein the Rab6a:Rab GGTase molar ratio is about 1:2.5.

12. The method of any one of claims 1-8, wherein the Rab6a:Rab GGTase molar ratio is 1:2.5.

13. The method of any one of claims 1-12, wherein the lipid donor substrate comprises geranylgeranylpyrophosphate (GGPP) or an analogue thereof.

14. The method of any one of claims 1-13, wherein the lipid donor substrate comprises biotin-geranylpyrophosphate (BGPP).

15. The method of any one of claims 1-14, wherein detecting the lipidated Rab6a comprises an enzyme-linked immunosorbent assay (ELISA), a Western blot analysis or an autoradiography.

16. The method of any one of claims 6-15, wherein the AAV vector comprising nucleotide sequence encoding the REP1 protein is manufactured for use in the treatment of choroideremia.

17. The method of any one of claims 6-16, wherein the lipidated Rab6a is detected and wherein the REP-1 protein or the AAV vector comprising nucleotide sequence encoding the REP1 protein is suitable for use in the treatment of choroideremia.

18. The method of any one of claims 1-17, wherein detecting the lipidated Rab6a further comprises quantifying an amount of the lipidated Rab6a.

19. The method of claim 18, wherein the amount of lipidated Rab6a is an absolute amount.

20. The method of claim 18, wherein the amount of lipidated Rab6a is a relative amount.

21. The method of claim 20, wherein the amount of lipidated Rab6a is relative to a control amount or to a reference level.

22. A use of a Rab6a protein for determining an activity of a Rab escort protein 1 (REP1) protein.

23. The use of claim 22, wherein the REP1 protein is isolated or derived from a cell and wherein the cell is genetically engineered to express the REP1 protein.

24. The use of claim 22, wherein a cell comprises the REP1 protein and wherein the cell is genetically engineered to express the REP1 protein.

25. The use of claim 22, wherein the REP1 protein is isolated or derived from a lysate of from a cell and wherein the cell is genetically-engineered to express the REP1 protein.

26. The use of claim 22, wherein a cell lysate comprises the REP1 protein, wherein the cell lysate is isolated or derived from a cell, and wherein the cell is genetically-engineered to express the REP1 protein.

27. The use of any one of claims 23-26, wherein the REP1 protein is expressed from a viral vector comprising a nucleotide sequence encoding the REP1 protein.

28. The use of claim 27, wherein the viral vector is an adeno-associated viral (AAV) vector.

29. The use of any one of claims 22-28, wherein the AAV vector comprising nucleotide sequence encoding the REP1 protein is manufactured for use in the treatment of choroideremia.

30. The use of any one of claims 22-29, wherein the lipidated Rab6a is detected and wherein the REP-1 protein or the AAV vector comprising nucleotide sequence encoding the REP1 protein is suitable for use in the treatment of choroideremia.

31. The use of any one of claims 22-30, wherein the Rab6a protein is substantially pure.
